(19) United States
(12) Patent Application Publication
(10) Pub. No.: US 2019/0125852 A1
(43) Pub. Date: May 2, 2019

(54) COMPOSITIONS AND METHODS FOR TUMOR VACCINATION USING PROSTATE CANCER-ASSOCIATED ANTIGENS

(71) Applicant: ETUBICS CORPORATION, Seattle, WA (US)

(72) Inventors: Frank R. JONES, Seattle, WA (US); Joseph BALINT, Seattle, WA (US); Yvette LATCHMAN, Seattle, WA (US); Adrian RICE, Seattle, WA (US); Elizabeth GABITZSCH, Seattle, WA (US)

(21) Appl. No.: 16/306,097

(22) PCT Filed: Jun. 2, 2017
(86) PCT No.: PCT/US17/35694

§ 371 Date: Nov. 30, 2018

Related U.S. Application Data
(60) Provisional application No. 62/345,582, filed on Jun. 3, 2016.

(51) Int. Cl. A61K 39/00; C12N 15/86; A61P 35/00

(52) U.S. Cl. CPC A61K 39/001194; A61K 39/001195; C12N 15/86; A61K 2039/70; A61K 39/00117; C12N 2710/10343; C12N 2710/10334; A61P 35/00

(57) ABSTRACT

Methods and compositions for constructing and producing recombinant adenovirus-based vector vaccines are provided. In particular aspects, there are be provided compositions and methods involving adenovirus vectors comprising genes for target antigens, such as pro state-specific antigen (PSA), pro state-specific membrane antigen (PSMA), MUC1, CEA, and/or Brachyury, and costimulatory molecules for use in treatment methods that generate highly reactive anti-tumor immune responses and that allows for multiple vaccinations in individuals with preexisting immunity to adenovirus.

### CROSS REFERENCE

This application claims the benefit of U.S. Provisional Patent Application No. 62/345,582 filed Jun. 3, 2016, the disclosure of which is herein incorporated by reference in its entirety.

### BACKGROUND

Vaccines help the body fight disease by training the immune system to recognize and destroy harmful substances and diseased cells. Vaccines can be largely grouped into two types, preventive and treatment vaccines. Prevention vaccines are given to healthy people to prevent the development of specific diseases, while treatment vaccines, also referred to as immunotherapies, are given to a person who has been diagnosed with disease to help stop the disease from growing and spreading or as a preventive measure.

Viral vaccines are currently being developed to help fight infectious diseases and cancers. These viral vaccines work by inducing expression of a small fraction of genes associated with a disease within the host's cells, which in turn, enhance the host's immune system to identify and destroy diseased cells. As such, clinical response of a viral vaccine can depend on the ability of the vaccine to obtain a high-level immunogenicity and have sustained long-term expression.

Therefore, there remains a need to discover novel compositions and methods for enhanced therapeutic response to complex diseases such as cancer.

### SUMMARY

In various aspects, the present disclosure provides a composition comprising a replication-defective virus vector comprising a nucleic acid sequence encoding a prostate specific antigen (PSA) and/or a nucleic acid sequence encoding prostate-specific membrane antigen (PSMA), wherein the PSA has an amino acid sequence at least 80%, at least 85%, at least 90%, at least 92%, at least 95%, at least 97%, or at least 99% identical with SEQ ID NO: 1 or SEQ ID NO: 34 or the PSMA has an amino acid sequence at least 80% identical with SEQ ID NO: 11.

In some aspects, the vector comprises a nucleic acid sequence encoding a PSA having an amino acid sequence at least 80%, at least 85%, at least 90%, at least 92%, at least 95%, at least 97%, or at least 99% identical with SEQ ID NO: 35 or the nucleic acid sequence encoding PSA has at least 80%, at least 85%, at least 90%, at least 92%, at least 95%, at least 97%, or at least 99% identical with SEQ ID NO: 2. In some aspects, the vector comprises a nucleic acid sequence encoding a PSMA having an amino acid sequence at least 80%, at least 85%, at least 90%, at least 92%, at least 95%, at least 97%, or at least 99% identical with SEQ ID NO: 36.

In some aspects, the composition further comprises a second replication-defective virus vector comprising a second nucleic acid sequence encoding a Brachyury antigen, a third replication-defective virus vector comprising a third nucleic acid sequence encoding a MUC1 antigen, or a combination thereof. In some aspects, the Brachyury antigen binds to HLA-A2, HLA-A3, HLA-A24, or a combination thereof. In some aspects, the Brachyury antigen is a modified Brachyury antigen comprising an amino acid sequence set forth in WLLPGTSTV (SEQ ID NO: 7). In some aspects, the Brachyury antigen is a modified Brachyury antigen comprising an amino acid sequence at least 80%, at least 85%, at least 90%, at least 92%, at least 95%, at least 97%, or at least 99% identical to SEQ ID NO: 5, SEQ ID NO: 6, or SEQ ID NO: 42 In some aspects, the second replication-defective vector comprises a nucleotide sequence at least 80%, at least 85%, at least 90%, at least 92%, at least 95%, at least 97%, or at least 99% identical with SEQ ID NO: 3, SEQ ID NO: 4, positions 13 to 1242 of SEQ ID NO: 4, SEQ ID NO: 42. In some aspects, the second replication-defective vector comprises a nucleotide sequence at least 80% identical, at least 85%, at least 90%, at least 92%, at least 95%, at least 97%, or at least 99% to SEQ ID NO: 12 (Ad vector with sequence encoding ma modified Brachyury antigen), positions 1033-2083 of SEQ ID NO: 12, or SEQ ID NO: 42.

In some aspects, the MUC1 antigen comprises a sequence at least 80%, at least 85%, at least 90%, at least 92%, at least 95%, at least 97%, or at least 99% identical to SEQ ID NO: 10 or SEQ ID NO: 41. In some aspects, the third nucleic acid sequence encoding a MUC1 antigen comprises at least 80%, at least 85%, at least 90%, at least 92%, at least 95%, at least 97%, or at least 99% identity to SEQ ID NO: 8, SEQ ID NO: 9, or SEQ ID NO: 41. In some aspects, the MUC-1 antigen binds to HLA-A2, HLA-A3, HLA-A24, or a combination thereof.

In other aspects, the replication-defective virus vector, the second replication-defective virus vector, and/or the third replication-defective virus vector is an adenovirus vector. In some aspects, the adenovirus vector comprises a deletion in an E1 region, an E2b region, an E3 region, an E4 region, or a combination thereof. In some aspects, the adenovirus vector comprises a deletion in an E2b region. In further aspects, the adenovirus vector comprises a deletion in an E1 region, an E2b region, and an E3 region.

In some aspects, the composition comprises from at least 1×10⁹ virus particles to at least 5×10¹² virus particles. In some aspects, the composition comprises at least 5×10⁹ virus particles. In some aspects, the composition comprises at least 5×10¹⁰ virus particles. In some aspects, the composition comprises at least 5×10¹¹ virus particles. In some aspects, the composition comprises at least 5×10¹² virus particles.

In some aspects, the composition or the replication-defective virus vector further comprises a nucleic acid sequences encoding a costimulatory molecule. In some aspects, the costimulatory molecule comprises B7, ICAM-1, LFA-3, or a combination thereof. In some aspects, the costimulatory molecule comprises a combination of B7, ICAM-1, and LFA-3.

In other aspects, the composition further comprises a plurality of nucleic acid sequences encoding a plurality of costimulatory molecules positioned in the same replication-defective virus vector. In some aspects, the composition further comprises a plurality of nucleic acid sequences encoding a plurality of costimulatory molecules positioned in separate replication-defective virus vectors.

In additional aspects, the composition further comprises a nucleic acid sequence encoding one or more additional target antigens or immunological epitopes thereof. In some aspects, the replication-defective virus vector further comprises a nucleic acid sequence encoding one or more additional target antigens or immunological epitopes thereof. In some aspects, the one or more additional target antigens is a tumor neo-antigen, tumor neo-epitope, tumor-specific antigen, tumor-associated antigen, tissue-specific antigen, bacterial antigen, viral antigen, yeast antigen, fungal antigen, protozoan antigen, parasite antigen, mitogen, or a combination thereof. In some aspects, the one or more additional target antigens is CEA, folate receptor alpha, WT1, HPV E6, HPV E7, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, PSCA, PSMA, PAP, Tyrosinase, TRP-1, TRP-2, ART-4, CAMEL, Cyp-B, Her2/neu, BRCA1, BRACHYURY, BRACHYURY (TIVS7-2, polymorphism), BRACHYURY (IVS7 T/C polymorphism), T BRACHYURY, T, hTERT, hTRT, iCE, MUC1, MUC1 (VNTR polymorphism), MUC1c, MUC1n, MUC2, PRAME, P15, RU1, RU2, SART-1, SART-3, WT1, AFP, β-catenin/m, Caspase-8/m, CDK-4/m, Her2/neu, Her3, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, or TEL/AML1, or a modified variant, a splice variant, a functional epitope, an epitope agonist, or a combination thereof. In some aspects, the one or more additional target antigens is CEA In some aspects, the one or more additional target antigens is CEA, Brachyury, and MUC1. In some aspects, CEA is at least 80%, at least 85%, at least 90%, at least 92%, at least 95%, at least 97%, or at least 99% identical to SEQ ID NO: 37 or SEQ ID NO: 38. In some aspects, the one or more additional target antigens is HER3. In some aspects, the one or more additional target antigens is HPV E6 or HPV E7.

In some aspects, the replication-defective virus vector further comprises a selectable marker. In some aspects, the selectable marker is a lacZ gene, thymidine kinase, gpt, GUS, or a vaccinia K1L host range gene, or a combination thereof.

In various aspects, the present disclosure provides a composition comprising one or more replication-defective virus vectors comprising a nucleic acid sequence encoding a prostate specific antigen (PSA), a nucleic acid sequence encoding prostate-specific membrane antigen (PSMA), a nucleic acid sequence encoding a Brachyury antigen, a nucleic acid sequence encoding a MUC1 antigen, or a combination thereof.

In various aspects, the present disclosure provides a composition comprising one or more replication-defective virus vectors comprising a nucleic acid sequence encoding a prostate specific antigen (PSA), a nucleic acid sequence encoding a Brachyury antigen, and a nucleic acid sequence encoding a MUC1 antigen.

In various aspects, the present disclosure provides a composition comprising one or more replication-defective virus vectors comprising a nucleic acid sequence encoding prostate-specific membrane antigen (PSMA), a nucleic acid sequence encoding a Brachyury antigen, and a nucleic acid sequence encoding a MUC1 antigen.

In various aspects, the present disclosure provides a composition comprising one or more replication-defective virus vectors comprising a nucleic acid sequence encoding a prostate specific antigen (PSA), a nucleic acid sequence encoding prostate-specific membrane antigen (PSMA), a nucleic acid sequence encoding a Brachyury antigen, a nucleic acid sequence encoding a MUC1 antigen, and a nucleic acid sequence encoding a CEA antigen.

In some aspects, the replication-defective virus vector of any of the above compositions further comprises a nucleic acid sequence encoding an immunological fusion partner.

In various aspects, the present disclosure provides a pharmaceutical composition comprising the composition according any composition described herein and a pharmaceutically acceptable carrier.

In various aspects, the present disclosure provides a host cell comprising the composition according to any composition described herein.

In various aspects, the present disclosure provides a method of preparing a tumor vaccine, the method comprising preparing a pharmaceutical composition according to claim 42. In various aspects, the present disclosure provides a method of enhancing an immune response in a subject in need thereof, the method comprising administering a therapeutically effective amount of any composition described herein or the pharmaceutical composition as described herein to the subject. In various aspects, the present disclosure provides a method of treating a PSA-expressing or PSMA-expressing cancer in a subject in need thereof, the method comprising administering a therapeutically effective amount of any composition described herein or the pharmaceutical composition as described herein to the subject.

In some aspects, the method further comprises readministering the pharmaceutical composition to the subject.

In some aspects, the method further comprises administering an immune checkpoint inhibitor to the subject. In further aspects, the immune checkpoint inhibitor inhibits PD1, PDL1, PDL2, CD28, CD80, CD86, CTLA4, B7RPI, ICOS, B7RPI, B7-H3, B7-H4, BTLA, HVEM, KIR, TCR, LAG3, CD137, CD137L, OX40, OX40L, CD27, CD70, CD40, CD40L, TIM3, GAL9, ADORA, CD276, VTCN1, IDO1, KIR3DL1, HAVCR2, VISTA, or CD244 In some aspects, the immune checkpoint inhibitor inhibits PD1 or PDL1 In some aspects, the immune checkpoint inhibitor is an anti-PD1 or anti-PDL1 antibody. In some aspects, the immune checkpoint inhibitor is an anti-PDL1 antibody.

In some aspects, a route of administration is intravenous, subcutaneous, intralymphatic, intratumoral, intradermal, intramuscular, intraperitoneal, intrarectal, intravaginal, intranasal, oral, via bladder instillation, or via scarification.

In some aspects, the enhanced immune response is a cell-mediated or humoral response. In some aspects, the enhanced immune response is an enhancement of B-cell proliferation, CD4+ T cell proliferation, CD8+ T cell proliferation, or a combination thereof. In some aspects, the enhanced immune response is an enhancement of IL-2 production, IFN-γ production or combination thereof. In some aspects, the enhanced immune response is an enhancement of antigen presenting cell proliferation, function or combination thereof.

In some aspects, the subject has been previously administered an adenovirus vector. In some aspects, the subject has pre-existing immunity to adenovirus vectors. In some aspects, the subject is determined to have pre-existing immunity to adenovirus vectors.

In some aspects, the method further comprises administering to the subject a chemotherapy, radiation, a different immunotherapy, or a combination thereof.

In some aspects, the subject is a human or a non-human animal. In some aspects, the subject has previously been treated for cancer.

In some aspects, the administering the therapeutically effective amount is repeated at least three times. In some aspects, the administering the therapeutically effective amount comprises 1×10⁹ to 5×10¹² virus particles per dose. In some aspects, the administering the therapeutically effective amount comprises 5×10⁹ virus particles per dose. In some aspects, the administering the therapeutically effective amount comprises 5×10¹⁰ virus particles per dose. In some aspects, the administering the therapeutically effective amount comprises 5×10¹¹ virus particles per dose. In some aspects, the administering the therapeutically effective amount comprises 5×10¹² virus particles per dose. In some aspects, the administering the therapeutically effective amount is repeated every one, two, or three weeks.

In some aspects, the administering the therapeutically effective amount is followed by one or more booster immunizations comprising the same composition or pharmaceutical composition. In some aspects, the booster immunization is administered every one, two, three, four, five, six, seven, eight, nine, ten, eleven, or twelve months or more. In some aspects, the booster immunization is repeated three four, five, six, seven, eight, nine, ten, eleven, or twelve or more times. In some aspects, the administering the therapeutically effective amount is a primary immunization repeated every one, two, or three weeks for three four, five, six, seven, eight, nine, ten, eleven, or twelve or more times followed by a booster immunization repeated every one, two, three, four, five, six, seven, eight, nine, ten, eleven, or twelve or more months for three or more times.

In additional aspects, the method further comprises administering to the subject a pharmaceutical composition comprising a population of engineered nature killer (NK) cells. In some aspects, the engineered NK cells comprise one or more NK cells that have been modified as essentially lacking the expression of MR (killer inhibitory receptors), one or more NK cells that have been modified to express a high affinity CD16 variant, and one or more NK cells that have been modified to express one or more CARs (chimeric antigen receptors), or any combinations thereof. In some aspects, the engineered NK cells comprise one or more NK cells that have been modified as essentially lacking the expression MR. In some aspects, the engineered NK cells comprise one or more NK cells that have been modified to express a high affinity CD16 variant. In some aspects, the engineered NK cells comprise one or more NK cells that have been modified to express one or more CARs. In further aspects, the CAR is a CAR for a tumor neo-antigen, tumor neo-epitope, WT1, HPV-E6, HPV-E7, p53, MAGE-A1, MAGE-A2, MAGE-A3, MAGE-A4, MAGE-A6, MAGE-A10, MAGE-A12, BAGE, DAM-6, DAM-10, Folate receptor alpha, GAGE-1, GAGE-2, GAGE-8, GAGE-3, GAGE-4, GAGE-5, GAGE-6, GAGE-7B, NA88-A, NY-ESO-1, MART-1, MC1R, Gp100, PSA, PSM, Tyrosinase, TRP-1, TRP-2, ART-4, CAMEL, CEA, Cyp-B, Her2/neu, Her3, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUC1n, MUC2, PRAME, P15, PSCA, PSMA, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, or any combination thereof.

In some aspects, a cell comprises the replication-defective adenovirus vector. In some aspects, the cell is a dendritic cells (DC).

In some aspects, the method further comprises administering a pharmaceutical composition comprises a therapeutically effective amount of IL-15 or a replication-defective vector comprising a nucleic acid sequence encoding IL-15.

In some aspects, the subject has prostate cancer. In some aspects, the subject has advanced stage prostate cancer. In some aspects, the subject has unresectable, locally advanced, or metastatic cancer.

In some aspects, the administering the therapeutically effective amount of any composition described herein or the pharmaceutical composition as described herein comprises a first replication-defective virus vector comprising a first nucleic acid sequence encoding a PSA antigen, a second replication-defective virus vector comprising a second nucleic acid sequence encoding a PSMA antigen, a third replication-defective virus vector comprising a third nucleic acid sequence encoding a Brachyury antigen, a fourth replication-defective virus vector comprising a fourth nucleic acid sequence encoding a MUC1 antigen at a 1:1:1:1 ratio.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates induction of PSA specific cellular immunity in mice after homologous immunizations.

FIG. 1A illustrates IFN-γ cellular mediated immune (CMI) response in Ad5 immune BALB/c mice immunized three times with either an injection buffer (control group) or 10¹⁰ Virus Particles (VP) of Ad5[E1−, E2b−]-PSA at 7 day intervals.

FIG. 1B illustrates IL-2 cellular mediated immune (CMI) response in Ad5 immune BALB/c mice immunized three times with either an injection buffer (control group) or 10¹⁰ Virus Particles (VP) of Ad5[E1−, E2b−]-PSA at 7 day intervals.

FIG. 2 illustrate specificity of PSA cellular mediated immunity following immunizations with Ad5 [E1−, E2b−]-PSA.

FIG. 2A illustrates IFN-γ spot forming cells (SFC) per 10⁶ splenocytes after ex vivo exposure to PSA or control antigens (HIV-gag, CMV).

FIG. 2B illustrates IL-2 spot forming cells (SFC) per 10⁶ splenocytes after ex vivo exposure to PSA or control antigens (HIV-gag, CMV).

FIG. 3 illustrates PSA directed antibody (anti-PSA Ab) responses using a quantitative ELISA.

FIG. 4 illustrates tumor growth in mice immunized with Ad5 [E1−, E2b−]-PSA compared to mice immunized with Ad5 [E1−, E2b−]-null following implantation of PSA expressing tumor cells.

FIG. 5 illustrates PSA secretion from cells after infection with Ad5 [E1−]-PSA or Ad5 [E1−, E2b−]-PSA. RM-11 murine prostate tumor cells or HEK-293 cells were infected with Ad5 [E1−]-PSA or Ad5 [E1−, E2b−]-PSA, respectively. Levels of PSA secreted into medium were assessed at various time points. Note the greater secretion of PSA by cells infected with Ad5 [E1−, E2b−]-PSA as compared to cells infected with Ad5 [E1−]-PSA.

FIG. 6 illustrates PSA specific cellular immunity in naïve mice after immunizing with Ad5 [E1−, E2b−]-PSA three times or Ad5-immune mice after immunizing with Ad5 [E1−, E2b−]-PSA three times. Naïve or Ad5-immune BALB/c mice were immunized three times with either injection buffer (control group) or 10¹⁰ VP of Ad5 [E1−, E2b−]-PSA at 7 day intervals. Splenocytes were assessed 14 days after the final immunization for the secretion of IFN-γ in an ELISpot assay. Cells were exposed to 2 μg of PSA antigen.

FIG. 7 illustrates PSA specific cellular immunity in naïve mice after immunizing with Ad5 [E1−, E2b−]-PSA three times or Ad5-immune mice mice after immunizing with Ad5 [E1-, E2b−]-PSA three times. Naïve or Ad5-immune BALB/c mice were immunized three time with either injection buffer (control group) or 10¹⁰ VP of Ad5 [E1−, E2b−]-PSA at 7 day intervals. Splenocytes were assessed 14 days after the final immunization for the secretion of IL-2 in an ELISpot assay. Cells were exposed to 2 μg of PSA antigen.

FIG. 8 illustrates specificity of PSA cellular mediated immunity following immunization with Ad5 [E1−, E2b−]-PSA in Ad5-immune mice. Ad5-immune BALB/c mice were immunized two times with 10¹⁰ VP of Ad5 [E1−]-null at a 14 day interval. Two weeks after the last immunization of Ad5 [E1−]-null, the mice were immunized three times with 10¹⁰ VP of Ad5 [E1−, E2b−]-PSA at 7 day intervals. Splenocytes were assessed by ELISpot assay 14 days after the final immunization for the secretion of both IFN-γ and IL-2 after ex vivo exposure to PSA or control antigens (HIV-gag, CMV).

FIG. 8A illustrates the frequency of IFN-γ secreting cells after ex vivo exposure of splenocytes to PSA or control antigen peptide pools (HIV-gag, CMV).

FIG. 8B illustrates the frequency of IL-2 secreting cells after ex vivo exposure of splenocytes to PSA or control antigen peptide pools (HIV-gag, CMV).

FIG. 9 illustrates anti-PSA antibody (Ab) activity in naïve mice after immunizing with Ad5 [E1−, E2b−]-PSA three times. BALB/c mice were immunized three time with either injection buffer (control group) or 10¹⁰ VP of Ad5 [E1−, E2b−]-PSA at 7 day intervals. Sera were assessed 14 days after the final immunization for the presence of anti-PSA Ab in a quantitative ELISA using purified PSA as an antibody capture antigen target.

FIG. 10 illustrates possible prostate cancer multi-antigen gene construct for insertion into Ad5 [E1−, E2b−].

FIG. 10A illustrates a triple gene insert for a prostate cancer vaccine.

FIG. 10B illustrates the products after translation of FIG. 10A.

FIG. 11 illustrates analysis of IFN-γ-, IL-2- and Granzyme B-expressing splenocytes following vaccination of mice with Ad5 [E1−, E2b−]-PSMA. C57BL/6 mice (n=5/group) were vaccinated twice at 2 week intervals with 10¹⁰ VP of Ad5 [E1−, E2b−]-PSMA (red bar) or Ad5 [E1−, E2b−]-null (black bar). Splenocytes were collected 7 days after the final vaccination and ex vivo exposure to a PSMA peptide pool, a negative control antigen (Nef peptide pool), or a positive control (ConA). An ELISPOT assay was used to evaluate IFN-γ secretion, IL-2 secretion, and Granzyme B secretion after exposure to PSMA peptide pools, a negative control antigen (Nef peptide pool), or ConA, respectively. Data are reported as the number of spot forming cells (SFs) per 10⁶ splenocytes. The error bars depict the SEM.

FIG. 11A illustrates the frequency of IFN-γ-secreting cells after ex vivo stimulation.

FIG. 11B illustrates the frequency of IL-2-secreting cells after ex vivo stimulation.

FIG. 11C illustrates the frequency of Granzyme B-secreting cells after ex vivo stimulation.

FIG. 12 illustrates analysis of CD8+ splenocytes and CD4+ splenocytes and multifunctional cellular populations following vaccination with Ad5 [E1−, E2b−]-PSMA. C57BL/6 mice (n=5/group) were vaccinated twice at 2 week intervals with 10¹⁰ VP of Ad5 [E1−, E2b−]-PSMA or Ad5 [E1−, E2b−]-null (black bar). Splenocytes were collected 7 days after the final vaccination and were stimulated ex vivo with a PSMA peptide pool or a negative control (plain media or SIV nef peptide pool). Cells were assessed by flow cytometry for phenotype and inflammatory cytokine secretion. For positive controls, splenocytes were exposed to PMA/ionomycin (data not shown). Error bars depict the SEM.

FIG. 12A illustrates the percentage of CD8β+ splenocytes secreting IFN-γ after ex vivo stimulation.

FIG. 12B illustrates the percentage of CD4+ splenocytes secreting IFN-γ after ex vivo stimulation.

FIG. 12C illustrates the percentage of CD8β+ splenocytes secreting IFN-γ and TNF-α after ex vivo stimulation.

FIG. 12D illustrates the percentage of CD4+ splenocytes secreting IFN-γ and TNF-α after ex vivo stimulation.

FIG. 13 illustrates antibody responses in mice following vaccination with Ad5 [E1−, E2b−]-PSMA. C57BL/6 mice (n=5/group) were vaccinated twice at 2 week intervals with 10¹⁰ VP of Ad5 [E1−, E2b−]-PSMA (red bar) or Ad5 [E1−, E2b−]-null (black bar). Sera were collected 7 days after the final vaccination and assessed by ELISA for antigen specific antibodies against PSMA protein.

FIG. 14 illustrates analysis of IFN-γ-, IL-2- and Granzyme B-expressing splenocytes following vaccination of mice with Ad5 [E1−, E2b−]-PSA. C57BL/6 mice (n=5/group) were vaccinated three times at 2 week intervals before the tumor was implanted with 10¹⁰ VP of Ad5 [E1−, E2b−]-PSA (striped bar) or Ad5 [E1−, E2b−]-null (black bar). Two weeks after the final vaccination, mice were injected with 5×10⁵ D2F2 tumorgenic cells that express PSA, into the right hind side of mice. Splenocytes were collected at the end of the experiment (37 days post-tumor implant) and stimulated ex vivo with a PSA peptide pool, a negative control (SIV-Nef peptide pool), or a positive control (Concanavalin A (Con A)). Cytokine secretion was measured after ex vivo stimulation using an ELISPOT assay. Data are reported as the number of spot forming cells (SFC) per 10⁶ splenocytes and error bars show the SEM.

FIG. 14A illustrates IFN-γ spot forming cells (SFC) per 10⁶ splenocytes after ex vivo exposure stimulation.

FIG. 14B illustrates IL-2 spot forming cells (SFC) per 10⁶ splenocytes after ex vivo stimulation.

FIG. 14C illustrates Granzyme B spot forming cells (SFC) per 10⁶ splenocytes after ex vivo stimulation.

FIG. 15 illustrates analysis of CD8+ splenocytes and CD4+ splenoctyes and multifunctional cellular populations following vaccination with Ad5 [E1−, E2b−]-PSA. C57BL/6 mice (n=5/group) were vaccinated three times at 2 week intervals with 10¹⁰ VP of Ad5 [E1−, E2b−]-PSA or Ad5 [E1−, E2b−]-null (black bar). Two weeks after the final vaccination, mice were injected with 5×10⁵ D2F2 tumorgenic cells that express PSA, into the right hind side of mice. Splenocytes were collected at the end of the experiment (37 days post-tumor inoculation) and exposed ex vivo to a PSA peptide pool or a negative control antigen (media or SIV-Nef peptide pool). Cells were stained for surface markers and for intracellular cytokine secretion and analyzed by flow cytometry.

FIG. 15A illustrates the percent of CD8β+ splenocytes secreting IFN-γ.

FIG. 15B illustrates the percent of CD4+ splenocytes secreting IFN-γ.

FIG. 15C illustrates the percent of CD8β+ splenocytes secreting IFN-γ and TNF-α.

FIG. 15D illustrates the percent of CD4+ splenocytes secreting IFN-γ and TNF-α.

FIG. 16 illustrates the antibody response measured in sera from BALB/c mice (n=5/group) immunized three times every two weeks with 10¹⁰ VPs of Ad5 [E1−, E2b−]-null or Ad5 [E1−, E2b−]-PSA. Two weeks after the final vaccination, mice were injected with 5×10⁵ D2F2 tumorgenic cells that express PSA, into the right hind side of mice. Sera was collected at the end of the experiment (37 days post-tumor inoculation) and analyzed for the presence of antibodies using an enzyme-linked immunosorbent assay (ELISA).

FIG. 16A illustrates the mass of IgG specific antibodies against PSA.

FIG. 16B illustrates the mass of IgG1 specific antibodies against PSA.

### DETAILED DESCRIPTION

The following passages describe different aspects of certain embodiments in greater detail. Each aspect may be combined with any other aspect or aspects unless clearly indicated to the contrary. In particular, any feature indicated as being preferred or advantageous may be combined with any other feature of features indicated as being preferred or advantageous.

Unless otherwise indicated, any embodiment can be combined with any other embodiment. A variety of aspects can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges include the range endpoints.

### I. Target Antigens

In certain aspects, there may be provided expression constructs or vectors comprising nucleic acid sequences that encode one or more target proteins of interest or target antigens, such as PSA, PSMA, CEA, MUC1, Brachyury, or a combination thereof as described herein. In this regard, there may be provided expression constructs or vectors that may contain nucleic acid encoding at least, at most or about one, two, three, four, five, six, seven, eight, nine, ten, 11, 12, 13, 14, 15, 16, 17, 18, 19, 20, 30, 40, 50, 60, 70, 80, 90, 100, 200, 300, 400, or 500 different target antigens of interest or any number or ranges derived therefrom. The expression constructs or vectors may contain nucleic acid sequences encoding multiple fragments or epitopes from one or more target antigens or may contain one or more fragments or epitopes from numerous different target antigens.

The target antigens may be a full-length protein or may be an immunogenic fragment (e.g., an epitope) thereof. Immunogenic fragments may be identified using available techniques, such as those summarized in Paul, Fundamental Immunology, 3rd ed., 243-247 (Raven Press, 1993) and references cited therein. Representative techniques for identifying immunogenic fragments include screening polypeptides for the ability to react with antigen-specific antisera and/or T-cell lines or clones. An immunogenic fragment of a particular target polypeptide may be a fragment that reacts with such antisera and/or T-cells at a level that is not substantially less than the reactivity of the full-length target polypeptide (e.g., in an ELISA and/or T-cell reactivity assay). In other words, an immunogenic fragment may react within such assays at a level that is similar to or greater than the reactivity of the full-length polypeptide. Such screens may generally be performed using methods available to those of ordinary skill in the art, such as those described in Harlow and Lane, Antibodies: A Laboratory Manual, Cold Spring Harbor Laboratory, 1988.

In some cases, a target antigen can be an immunogenic epitope, for example, an epitope of 8 to 10 amino acids long. In some cases, a target antigen is four to ten amino acids long or over 10 amino acids long. A target antigen can comprise a length of or can comprise a length of at least, about, or at most 1, 2, 3, 4, 5, 6, 7, 8, 9, 10, 11, 12, 13, 14, 15, 16, 17, 18, 19, 20 amino acids, or any number or ranges derived therefrom. A target antigen can be any length of amino acids.

Additional non-limiting examples of target antigens include carcinoembryonic antigen (CEA), folate receptor alpha, WT1, brachyury (TIVS7-2, polymorphism), brachyury (IVS7 T/C polymorphism), T brachyury, T, hTERT, hTRT, iCE, HPV E6, HPV E7, BAGE, DAM-6, -10, GAGE-1, -2, -8, GAGE-3, -4, -5, -6, -7B, NA88-A, NY-ESO-1, MART-1, MC1R, Gp100, PSA, PSMA, PSCA, STEAP, PAP, Tyrosinase, TRP-1, TRP-2, ART-4, CAMEL, Cyp-B, EGFR, Her2/neu, Her3, MUC1, MUC1 (VNTR polymorphism), MUC1-c, MUC1-n, MUC1, MUC2, PRAME, P15, RU1, RU2, SART-1, SART-3, WT1, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARa, TEL/AML1, human epidermal growth factor receptor 2 (HER2/neu), human epidermal growth factor receptor 3 (HER3), Human papillomavirus (HPV), Prostate-specific antigen (PSA), alpha-actinin-4, ARTC1, CAR-ABL fusion protein (b3a2), B-RAF, CASP-5, CASP-8, beta-catenin, Cdc27, CDK4, CDKN2A, COA-1, dek-can fusion protein, EFTUD2, Elongation factor 2, ETV6-AML1 fusion protein, FLT3-ITD, FN1, GPNMB, LDLR-fucosyltransferase fusion protein, HLA-A2d, HLA-A1 1d, hsp70-2, KIAAO205, MART2, ME1, neo-PAP, Myosin class I, NFYC, OGT, OS-9, pml-RARalpha fusion protein, PRDXS, PTPRK, K-ras, N-ras, RBAF600, SIRT2, SNRPD1, SYT-SSX1- or -SSX2 fusion protein, TGF-betaRII, triosephosphate isomerase, BAGE-1, GAGE-1, 2, 8, Gage 3, 4, 5, 6, 7, GnTVf, HERV-K-MEL, KK-LC-1, KM-HN-1, LAGE-1, MAGE-A1, MAGE-A2, MAGE-A3, MAGE-A4, MAGE-A6, MAGE-A9, MAGE-A10, MAGE-A12, MAGE-C2, mucink, NA-88, NY-ESO-1/LAGE-2, SAGE, Sp17, SSX-2, SSX-4, TAG-1, TAG-2, TRAG-3, TRP2-INT2g, XAGE-1b, gp100/Pme117, Kallikrein 4, mammaglobin-A, Melan-AJMART-1, NY-BR-1, OA1, PSA, RAB38/NY-MEL-1, TRP-1/gp75, TRP-2, tyrosinase, adipophilin, AIM-2, ALDH1A1, BCLX (L), BCMA, BING-4, CPSF, cyclin D1, DKK1, ENAH (hMena), EP-CAM, EphA3, EZH2, FGFS, G250/MN/CAIX, HER-2/neu, IL13Ralpha2, intestinal carboxyl esterase, alpha fetoprotein, M-CSFT, MCSP, mdm-2, MMP-2, MUC1, p53, PBF, PRAME, PSMA, RAGE-1, RGS5, RNF43, RU2AS, secernin 1, SOX10, STEAP1, survivin, Telomerase, VEGF, or any combination thereof.

In some aspects, tumor neo-epitopes as used herein are tumor-specific epitopes, such as EQVWGMAVR (SEQ ID NO: 13) or CQGPEQVWGMAVREL (SEQ ID NO: 14) (R346W mutation of FLRT2), GETVTMPCP (SEQ ID NO: 15) or NVGETVTMPCPKVFS (SEQ ID NO: 16) (V73M mutation of VIPR2), GLGAQCSEA (SEQ ID NO: 17) or NNGLGAQCSEAVTLN (SEQ ID NO: 18) (R286C mutation of FCRL1), RKLTTELTI (SEQ ID NO: 19), LGPERRKLTTELTII (SEQ ID NO: 20), or PERRKLTTE (SEQ ID NO: 21) (S1613L mutation of FAT4), MDWVWMDTT (SEQ ID NO: 22), AVMDWVWMDTTLSLS (SEQ ID NO: 23), or VWMDTTLSL (SEQ ID NO: 24) (T2356M mutation of PIEZO2), GKTLNPSQT (SEQ ID NO: 25), SWFREGKTLNPSQTS (SEQ ID NO: 26), or REGKTLNPS (SEQ ID NO: 27) (A292T mutation of SIGLEC14), VRNATSYRC (SEQ ID NO: 28), LPNVTVRNATSYRCG (SEQ ID NO: 29), or NVTVRNATS (SEQ ID NO: 30) (D1143N mutation of SIGLEC1), FAMAQIPSL (SEQ ID NO: 31), PFAMAQIPSLSLRAV (SEQ ID NO: 32), or AQIPSLSLR (SEQ ID NO: 33) (Q678P mutation of SLC4A11).

Tumor-associated antigens may be antigens not normally expressed by the host; they can be mutated, truncated, misfolded, or otherwise abnormal manifestations of molecules normally expressed by the host; they can be identical to molecules normally expressed but expressed at abnormally high levels; or they can be expressed in a context or environment that is abnormal. Tumor-associated antigens may be, for example, proteins or protein fragments, complex carbohydrates, gangliosides, haptens, nucleic acids, other biological molecules or any combinations thereof.

### II. PSA Family Antigen Targets

Disclosed herein include compositions comprising replication-defective vectors comprising one or more nucleic acid sequences encoding PSA and/or PSMA antigen, and/or one or more nucleic acid sequences encoding mucin family antigen such as MUC1, and/or one or more nucleic acid sequences encoding Brachyury, and/or one or more nucleic acid sequences encoding CEA in the same or separate replication-defective vectors.

Prostate-specific antigen (PSA), also known as gamma-seminoprotein or kallikrein-3 (KLK3), is a glycoprotein enzyme encoded in humans by the KLK3 gene. PSA is a member of the kallikrein-related peptidase family and is secreted by the epithelial cells of the prostate gland. PSA is produced for the ejaculate, where it liquefies semen in the seminal coagulum and allows sperm to swim freely. It is also believed to be instrumental in dissolving cervical mucus, allowing the entry of sperm into the uterus.

PSA is present in small quantities in the serum of men with healthy prostates, but is often elevated in the presence of prostate cancer or other prostate disorders. PSA is not a unique indicator of prostate cancer, but may also detect prostatitis or benign prostatic hyperplasia. Thirty percent of patients with high PSA have prostate cancer diagnosed after biopsy.

Targeting PSA and initiating a therapy based on its tumorigenicity is currently feasible because reliable testing can rapidly confirm the presence of elevated PSA levels in circulation and in human cancer biopsy. PSA is considered to be attractive antigenic target for tumor specific immunotherapy because the prostate cancer cells over express this antigen and elevated levels of PSA are associated with a diagnosis of prostate cancer. Studies indicate that PSA induced immune responses are effective at inducing anti-tumor CMI responses in humans and in experimental animal models of PSA expressing cancer.

Here disclosed include the use of an Ad5 [E1−, E2b−]-based vector platform to insert the human PSA gene as a new immunotherapy vaccine (referred to as Ad5 [E1−, E2b−]-PSA) to treat PSA expressing prostate cancers. In pre-clinical studies described in certain embodiments, this vaccine induced anti-tumor cell mediated immune (CMI) responses in a mouse model of PSA expressing cancer and provides us with a strong rationale for using the Ad5 [E1−, E2b−]-PSA as an immunotherapeutic vaccine to treat PSA expressing prostate cancers.

In some embodiments, a PSA antigen of this disclosure can have an amino sequence that is at least 80%, at least 85%, at least 90%, at least 92%, at least 95%, at least 97%, or at least 99% identical to SEQ ID NO: 34. In certain embodiments, a PSA antigen of this disclosure can have an amino acid sequence as set forth in SEQ ID NO: 34. In some embodiments, a PSA antigen of this disclosure can have a nucleotidesequence that is at least 80%, at least 85%, at least 90%, at least 92%, at least 95%, at least 97%, or at least 99% identical to SEQ ID NO: 35. In certain embodiments, a PSA antigen of this disclosure can have a nucleotide acid sequence as set forth in SEQ ID NO: 35.

### III. PSMA Antigen Targets

Disclosed herein include compositions comprising replication-defective vectors comprising one or more nucleic acid sequences encoding PSA and/or PSMA antigen, and/or one or more nucleic acid sequences encoding mucin family antigen such as MUC1, and/or one or more nucleic acid sequences encoding Brachyury, and/or one or more nucleic acid sequences encoding CEA in the same or separate replication-defective vectors.

Glutamate carboxypeptidase II (GCPII), also known as N-acetyl-L-aspartyl-L-glutamate peptidase I (NAALADase I), NAAG peptidase, or prostate-specific membrane antigen (PSMA) is an enzyme that in humans is encoded by the FOLH1 (folate hydrolase 1) gene. Human GCPII contains 750 amino acids and weighs approximately 84 kDa.

GCPII is a zinc metalloenzyme that resides in membranes. Most of the enzyme resides in the extracellular space. GCPII is a class II membrane glycoprotein. It catalyzes the hydrolysis of N-acetylaspartylglutamate (NAAG) to glutamate and N-acetylaspartate (NAA) according to the reaction scheme to the right.

In some embodiments, a PSMA antigen of this disclosure can have an amino sequence that is at least 80%, at least 85%, at least 90%, at least 92%, at least 95%, at least 97%, or at least 99% identical to SEQ ID NO: 11. In certain embodiments, a PSMA antigen of this disclosure can have an amino acid sequence as set forth in SEQ ID NO: 11. In some embodiments, a PSMA antigen of this disclosure can have a nucleotidesequence that is at least 80%, at least 85%, at least 90%, at least 92%, at least 95%, at least 97%, or at least 99% identical to SEQ ID NO: 36. In certain embodiments, a PSMA antigen of this disclosure can have a nucleotide acid sequence as set forth in SEQ ID NO: 36.

### IV. Mucin Family Antigen Targets

Disclosed herein include compositions comprising replication-defective vectors comprising one or more nucleic acid sequences encoding PSA and/or PSMA antigen, and/or one or more nucleic acid sequences encoding mucin family antigen such as MUC1, and/or one or more nucleic acid sequences encoding Brachyury, and/or one or more nucleic acid sequences encoding CEA in the same or separate replication-defective vectors.

The human mucin family (MUC1 to MUC21) includes secreted and transmembrane mucins that play a role in forming protective mucous barriers on epithelial surfaces in the body. These proteins function in to protecting the epithelia lining the respiratory, gastrointestinal tracts, and lining ducts in important organs such as, for example the mammary gland, liver, stomach, pancreas, and kidneys.

MUC1 (CD227) is a TAA that is over-expressed on a majority of human carcinomas and several hematologic malignancies. MUC1 (GenBank: X80761.1, NCBI: NM_001204285.1) and activates many important cellular pathways known to be involved in human disease. MUC1 is a heterodimeric protein formed by two subunits that is commonly overexpressed in several human cancers. MUC1 undergoes autoproteolysis to generate two subunits MUC1n and MUC1c that, in turn, form a stable noncovalent heterodimer.

The MUC1 C-terminal subunit (MUC1c) can comprise a 58 aa extracellular domain (ED), a 28 aa transmembrane domain (TM) and a 72 aa cytoplasmic domain (CD). The MUC1c also can contain a “CQC” motif that can allow for dimerization of MUC1 and it can also impart oncogenic function to a cell. In some cases, MUC1 can in part oncogenic function through inducing cellular signaling via MUC1c. MUC1c can interact with EGFR, ErbB2 and other receptor tyrosine kinases and contributing to the activation of the PI3K→AKT and MEK→ERK cellular pathways. In the nucleus, MUC1c activates the Wnt/β-catenin, STAT, and NF-κB RelA cellular pathways. In some cases MUC1 can impart oncogenic function through inducing cellular signaling via MUC1n. The MUC1 N-terminal subunit (MUC1n) can comprise variable numbers of 20 amino acid tandem repeats that can be glycosylated. MUC1 is normally expressed at the surface of glandular epithelial cells and is over-expressed and aberrantly glycosylated in carcinomas. MUC1 is a TAA that can be utilized as a target for tumor immunotherapy. Several clinical trials have been and are being performed to evaluate the use of MUC1 in immunotherapeutic vaccines. Importantly, these trials indicate that immunotherapy with MUC1 targeting is safe and may provide survival benefit.

However, clinical trials have also shown that MUC1 is a relatively poor immunogen. To overcome this, the inventors have identified a T lymphocyte immune enhancer peptide sequence in the C terminus region of the MUC1 oncoprotein (MUC1-C or MUC1c). Compared with the native peptide sequence, the agonist in their modified MUC1-C (a) bound HLA-A2 at lower peptide concentrations, (b) demonstrated a higher avidity for HLA-A2, (c) when used with antigen-presenting cells, induced the production of more IFN-γ by T-cells than with the use of the native peptide, and (d) was capable of more efficiently generating MUC1-specific human T-cell lines from cancer patients. Importantly, T-cell lines generated using the agonist epitope were more efficient than those generated with the native epitope for the lysis of targets pulsed with the native epitope and in the lysis of HLA-A2 human tumor cells expressing MUC1. Additionally, the inventors have identified additional CD8+ cytotoxic T lymphocyte immune enhancer agonist sequence epitopes of MUC1-C.

In certain aspects, there is provided a potent MUC1-C modified for immune enhancer capability (mMUC1-C or MUC1-C or MUC1c). The present disclosure provides a potent MUC1-C modified for immune enhancer capability incorporated it into a recombinant Ad5 [E1−, E2b−] platform to produce a new and more potent immunotherapeutic vaccine. For example, the immunotherapeutic vaccine can be Ad5 [E1−, E2b−]-mMUC1-C for treating MUC1 expressing cancers or infectious diseases.

Post-translational modifications play an important role in controlling protein function in the body and in human disease. For example, in addition to proteolytic cleavage discussed above, MUC1 can have several post-translational modifications such as glycosylation, sialylation, palmitoylation, or a combination thereof at specific amino acid residues. Provided herein are immunotherapies targeting glycosylation, sialylation, phosphorylation, or palmitoylation modifications of MUC1.

MUC1 can be highly glycosylated (N- and O-linked carbohydrates and sialic acid at varying degrees on serine and threonine residues within each tandem repeat, ranging from mono- to penta-glycosylation). Differentially O-glycosylated in breast carcinomas with 3,4-linked GlcNAc. N-glycosylation consists of high-mannose, acidic complex-type and hybrid glycans in the secreted form MUC1/SEC, and neutral complex-type in the transmembrane form, MUC1/TM.4. The present disclosure provides for immunotherapies targeting differentially O-glycosylated forms of MUC1.

Further, MUC1 can be sialylated. Membrane-shed glycoproteins from kidney and breast cancer cells have preferentially sialyated core 1 structures, while secreted forms from the same tissues display mainly core 2 structures. The O-glycosylated content is overlapping in both these tissues with terminal fucose and galactose, 2- and 3-linked galactose, 3- and 3,6-linked GalNAc-ol and 4-linked GlcNAc predominating. The present disclosure provides for immunotherapies targeting various sialylation forms of MUC1. Dual palmitoylation on cysteine residues in the CQC motif is required for recycling from endosomes back to the plasma membrane. The present disclosure provides for immunotherapies targeting various palmitoylation forms of MUC1.

Phosphorylation can affect MUC1's ability to induce specific cell signaling responses that are important for human health. The present disclosure provides for immunotherapies targeting various phosphorylated forms of MUC1. For example, MUC1 can be phosphorylated on tyrosine and serine residues in the C-terminal domain. Phosphorylation on tyrosines in the C-terminal domain can increase nuclear location of MUC1 and f3-catenin. Phosphorylation by PKC delta can induce binding of MUC1 to β-catenin/CTNNB1 and decrease formation of β-catenin/E-cadherin complexes. Src-mediated phosphorylation of MUC1 can inhibit interaction with GSK3B. Src- and EGFR-mediated phosphorylation of MUC1 on Tyr-1229 can increase binding to β-catenin/CTNNB1. GSK3B-mediated phosphorylation of MUC1 on Ser-1227 can decrease this interaction, but restores the formation of the β-cadherin/E-cadherin complex. PDGFR-mediated phosphorylation of MUC1 can increase nuclear colocalization of MUC1CT and CTNNB1. The present disclosure provides for immunotherapies targeting different phosphorylated forms of MUC1, MUC1c, and MUC1n known to regulate its cell signaling abilities.

The disclosure provides for immunotherapies that modulate MUC1c cytoplasmic domain and its functions in the cell. The disclosure provides for immunotherapies that comprise modulating a CQC motif in MUC1c. The disclosure provides for immunotherapies that comprise modulating the extracellular domain (ED), the transmembrane domain (TM), the cytoplasmic domain (CD) of MUC1c, or a combination thereof. The disclosure provides for immunotherapies that comprise modulating MUC1c's ability to induce cellular signaling through EGFR, ErbB2, or other receptor tyrosine kinases. The disclosure provides for immunotherapies that comprise modulating MUC1c's ability to induce PI3K→AKT, MEK→ERK, Wnt/β-catenin, STAT, NF-κB RelA cellular pathways, or combination thereof.

In some embodiments, the MUCic immunotherapy can further comprise PSA, PSMA, CEA, or Brachyury immunotherapy in the same replication-defective virus vectors or separate replication-defective virus vectors.

The disclosure also provides for immunotherapies that modulate MUCin and its cellular functions. The disclosure also provides for immunotherapies comprising tandem repeats of MUC1n, the glycosylation sites on the tandem repeats of MUC1n, or a combination thereof. In some embodiments, the MUC1n immunotherapy further comprises PSA, PSMA, CEA, or Brachyury immunotherapy in the same replication-defective virus vectors or separate replication-defective virus vectors.

The disclosure also provides vaccines comprising MUC1n, MUC1c, PSA, brachyury, CEA, or a combination thereof. The disclosure provides vaccines comprising MUC1c and PSA, PSMA, brachyury, CEA, or a combination thereof. The disclosure also provides vaccines targeting MUC1n and PSA, Brachyury, CEA, or a combination thereof. In some embodiments, the antigen combination is contained in one vector as provided herein. In some embodiments, the antigen combination is contained in a separate vector as provided herein.

The present invention relates to a replication defective adenovirus vector of serotype 5 comprising a sequence encoding an immunogenic polypeptide. The immunogenic polypeptide may be an isoform of MUC1 or a subunit or a fragment thereof. In some embodiments, the replication defective adenovirus vector comprises a sequence encoding a polypeptide with at least 75%, 80%, 85%, 90%, 95%, 98%, 99%, 99.5%, or 99.9% identity to the immunogenic polypeptide. In some embodiments, the immunogenic polypeptide encoded by the adenovirus vectors described herein comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human MUC1 sequence.

In some embodiments, a MUC1-c antigen of this disclosure can be a modified MUC1 and can have an amino acid sequence that is at least 80%, at least 85%, at least 90%, at least 92%, at least 95%, at least 97%, or at least 99% identical to SEQ ID NO: 10. In certain embodiments, a MUC1-c antigen of this disclosure can have an amino acid sequence as set forth in SEQ ID NO: 10. In some embodiments, a MUC1-c antigen of this disclosure can be a modified MUC1 and can have a nucleotide sequence that is at least 80%, at least 85%, at least 90%, at least 92%, at least 95%, at least 97%, or at least 99% identical to SEQ ID NO: 41. In certain embodiments, a MUC1-c antigen of this disclosure can have a nucleotide sequence as set forth in SEQ ID NO: 41.

### V. Brachyury Antigen Targets

Disclosed herein include compositions comprising replication-defective vectors comprising one or more nucleic acid sequences encoding PSA and/or PSMA antigen, and/or one or more nucleic acid sequences encoding mucin family antigen such as MUC1, and/or one or more nucleic acid sequences encoding Brachyury, and/or one or more nucleic acid sequences encoding CEA in the same or separate replication-defective vectors.

The disclosure provides for immunotherapies that comprise one or more antigens to Brachyury. Brachyury (also known as the “T” protein in humans) is a member of the T-box family of transcription factors that play key roles during early development, mostly in the formation and differentiation of normal mesoderm and is characterized by a highly conserved DNA-binding domain designated as T-domain. The epithelial to mesenchymal transition (EMT) is a key step during the progression of primary tumors into a metastatic state in which Brachyury plays a crucial role. The expression of Brachyury in human carcinoma cells induces changes characteristic of EMT, including up-regulation of mesenchymal markers, down-regulation of epithelial markers, and an increase in cell migration and invasion. Conversely, inhibition of Brachyury resulted in down-regulation of mesenchymal markers and loss of cell migration and invasion and diminished the ability of human tumor cells to form metastases. Brachyury can function to mediate epithelial-mesenchymal transition and promotes invasion.

The disclosure also provides for immunotherapies that modulate Brachyury effect on epithelial-mesenchymal transition function in cell proliferation diseases, such as cancer. The disclosure also provides immunotherapies that modulate Brachyury's ability to promote invasion in cell proliferation diseases, such as cancer. The disclosure also provides for immunotherapies that modulate the DNA binding function of T-box domain of Brachyury. In some embodiments, the Brachyury immunotherapy can further comprise one or more antigens to PSA, PSMA, CEA, or MUC1, MUC1c or MUC1n.

Brachyury expression is nearly undetectable in most normal human tissues and is highly restricted to human tumors and often overexpressed making it an attractive target antigen for immunotherapy. In humans, Brachyury is encoded by the T gene (GenBank: AJ001699.1, NCBI: NM_003181.3). There are at least two different isoforms produced by alternative splicing found in humans. Each isoform has a number of natural variants.

Brachyury is immunogenic and Brachyury-specific CD8+ T-cells expanded in vitro can lyse Brachyury expressing tumor cells. These features of Brachyury make it an attractive tumor associated antigen (TAA) for immunotherapy. The Brachyury protein is a T-box transcription factor. It can bind to a specific DNA element, a near palindromic sequence “TCACACCT” through a region in its N-terminus, called the T-box to activate gene transcription when bound to such a site.

The disclosure also provides vaccines comprising Brachyury, PSA, PSMA, MUC1, CEA, or a combination thereof. In some embodiments, the antigen combination is contained in one vector as provided herein. In some embodiments, the antigen combination is contained in a separate vector as provided herein.

In particular embodiments, the present invention relates to a replication defective adenovirus vector of serotype 5 comprising a sequence encoding an immunogenic polypeptide. The immunogenic polypeptide may be an isoform of Brachyury or a subunit or a fragment thereof. In some embodiments, the replication defective adenovirus vector comprises a sequence encoding a polypeptide with at least 70%, 75%, 80%, 85%, 90%, 95%, 98%, 99%, 99.5%, or 99.9% identity to the immunogenic polypeptide. In some embodiments, the immunogenic polypeptide encoded by the adenovirus vectors described herein comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human Brachyury sequence.

In some embodiments, a Brachyury antigen of this disclosure can have an amino sequence that is at least 80%, at least 85%, at least 90%, at least 92%, at least 95%, at least 97%, or at least 99% identical to SEQ ID NO: 42. In certain embodiments, a Brachyury antigen of this disclosure can have an amino acid sequence as set forth in SEQ ID NO: 42.

### VI. CEA Antigen Targets

Disclosed herein include compositions comprising replication-defective vectors comprising one or more nucleic acid sequences encoding PSA and/or PSMA antigen, and/or one or more nucleic acid sequences encoding mucin family antigen such as MUC1, and/or one or more nucleic acid sequences encoding Brachyury, and/or one or more nucleic acid sequences encoding CEA in the same or separate replication-defective vectors.

CEA represents an attractive target antigen for immunotherapy since it is over-expressed in nearly all colorectal cancers and pancreatic cancers, and is also expressed by some lung and breast cancers, and uncommon tumors such as medullary thyroid cancer, but is not expressed in other cells of the body except for low-level expression in gastrointestinal epithelium. CEA contains epitopes that may be recognized in an MHC restricted fashion by T-cells.

It was discovered that multiple homologous immunizations with Ad5 [E1−, E2b−]-CEA(6D), encoding the tumor antigen CEA, induced CEA-specific cell-mediated immune (CMI) responses with antitumor activity in mice despite the presence of pre-existing or induced Ad5-neutralizing antibody. In the present phase I/II study, cohorts of patients with advanced colorectal cancer were immunized with escalating doses of Ad5 [E1−, E2b−]-CEA(6D). CEA-specific CMI responses were observed despite the presence of pre-existing Ad5 immunity in a majority (61.3%) of patients. Importantly, there was minimal toxicity, and overall patient survival (48% at 12 months) was similar regardless of pre-existing Ad5 neutralizing antibody titers. The results demonstrate that, in cancer patients, the novel Ad5 [E1−, E2b−] gene delivery platform generates significant CMI responses to the tumor antigen CEA in the setting of both naturally acquired and immunization-induced Ad5 specific immunity.

CEA antigen specific CMI can be, for example, greater than 10, 20, 30, 40, 50, 100, 200, 300, 400, 500, 600, 700, 800, 900, 1000, 5000, 10000, or more IFN-γ spot forming cells (SFC) per 10⁶ peripheral blood mononuclear cells (PBMC). In some embodiments, the immune response is raised in a human subject with a preexisting inverse Ad5 neutralizing antibody titer of greater than 50, 100, 150, 200, 300, 400, 500, 600, 700, 800, 900, 1000, 1500, 2000, 2500, 3000, 3500, 4000, 4500, 5000, 6000, 7000, 8000, 9000, 1000, 12000, 15000, or higher. The immune response may comprise a cell-mediated immunity and/or a humoral immunity as described herein. The immune response may be measured by one or more of intracellular cytokine staining (ICS), ELISpot, proliferation assays, cytotoxic T-cell assays including chromium release or equivalent assays, and gene expression analysis using any number of polymerase chain reaction (PCR) or RT-PCR based assays, as described herein and to the extent they are available to a person skilled in the art, as well as any other suitable assays known in the art for measuring immune response.

In some embodiments, the replication defective adenovirus vector comprises a modified sequence encoding a subunit with at least 75%, 80%, 85%, 90%, 95%, 98%, 99%, 99.5%, or 99.9% identity to a wild-type subunit of the polypeptide.

The immunogenic polypeptide may be a mutant CEA or a fragment thereof. In some embodiments, the immunogenic polypeptide comprises a mutant CEA with an Asn->Asp substitution at position 610. In some embodiments, the replication defective adenovirus vector comprises a sequence encoding a polypeptide with at least 75%, 80%, 85%, 90%, 95%, 98%, 99%, 99.5%, or 99.9% identity to the immunogenic polypeptide. In some embodiments, the sequence encoding the immunogenic polypeptide comprises the sequence of SEQ ID NO: 37 (nucleic acid sequence for CEA-CAP1(6D)) or SEQ ID NO: 38 (amino acid sequence for the mutated CAP1(6D) epitope).

In some embodiments, the sequence encoding the immunogenic polypeptide comprises a sequence with at least 70% 75%, 80%, 85%, 90%, 95%, 98%, 99%, 99.5%, or 99.9% identity to SEQ ID NO: 37 or SEQ ID NO: 38 or a sequence generated from SEQ ID NO: 37 or SEQ ID NO: 38 by alternative codon replacements. In some embodiments, the immunogenic polypeptide encoded by the adenovirus vectors comprise up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human CEA sequence.

In some embodiments, the immunogenic polypeptide comprises a sequence from SEQ ID NO: 37 or a modified version, e.g., comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, of SEQ ID NO: 37 or SEQ ID NO: 38.

Members of the CEA gene family are subdivided into three subgroups based on sequence similarity, developmental expression patterns and their biological functions: the CEA-related Cell Adhesion Molecule (CEACAM) subgroup containing twelve genes (CEACAM1, CEACAM3-CEACAM8, CEACAM16 and CEACAM18-CEACAM21), the Pregnancy Specific Glycoprotein (PSG) subgroup containing eleven closely related genes (PSG1-PSG11) and a subgroup of eleven pseudogenes (CEACAMP1-CEACAMP11). Most members of the CEACAM subgroup have similar structures that consist of an extracellular Ig-like domains composed of a single N-terminal V-set domain, with structural homology to the immunoglobulin variable domains, followed by varying numbers of C2-set domains of A or B subtypes, a transmembrane domain and a cytoplasmic domain. There are two members of CEACAM subgroup (CEACAM16 and CEACAM20) that show a few exceptions in the organization of their structures. CEACAM16 contains two Ig-like V-type domains at its N and C termini and CEACAM20 contains a truncated Ig-like V-type 1 domain. The CEACAM molecules can be anchored to the cell surface via their transmembrane domains (CEACAM5 thought CEACAM8) or directly linked to glycophosphatidylinositol (GPI) lipid moiety (CEACAM5, CEACAM18 thought CEACAM21).

CEA family members are expressed in different cell types and have a wide range of biological functions. CEACAMs are found prominently on most epithelial cells and are present on different leucocytes. In humans, CEACAM1, the ancestor member of CEA family, is expressed on the apical side of epithelial and endothelial cells as well as on lymphoid and myeloid cells. CEACAM1 mediates cell-cell adhesion through hemophilic (CEACAM1 to CEACAM1) as well as heterothallic (e.g., CEACAM1 to CEACAM5) interactions. In addition, CEACAM1 is involved in many other biological processes, such as angiogenesis, cell migration, and immune functions. CEACAM3 and CEACAM4 expression is largely restricted to granulocytes, and they are able to convey uptake and destruction of several bacterial pathogens including Neisseria, Moraxella, and Haemophilus species.

Thus, in various embodiments, compositions and methods relate to raising an immune response against a CEA, selected from the group consisting of CEACAM1, CEACAM3, CEACAM4, CEACAM5, CEACAM6, CEACAM1, CEACAM8, CEACAM16, CEACAM18, CEACAM19, CEACAM20, CEACAM21, PSG1, PSG2, PSG3, PSG4, PSG5, PSG6, PSG7, PSG8, PSG9, and PSG11. An immune response may be raised against cells, e.g. cancer cells, expressing or overexpressing one or more of the CEAs, using the methods and compositions. In some embodiments, the overexpression of the one or more CEAs in such cancer cells is over 5, 10, 20, 30, 40, 50, 60, 70, 80, 90, 100 fold or more compared to non-cancer cells.

In certain embodiments, the CEA antigen used herein is a wild-type CEA antigen or a modified CEA antigen having a least a mutation in YLSGANLNL (SEQ ID NO: 39), a CAP1 epitope of CEA. The mutation can be conservative or non-conservative, substitution, addition, or deletion. In certain embodiments, the CEA antigen used herein has an amino acid sequence set forth in YLSGADLNL (SEQ ID NO: 38), a mutated CAP1 epitope. In further embodiments, the first replication-defective vector or a replication-defective vectors that express CEA has a nucleotide sequence at least 50%, 60%, 65%, 70%, 75%, 80%, 85%, 90%, 95%, 98%, 99%, 99.5%, 99.9%, or 100% identical to any portion of SEQ ID NO: 40 (the predicted sequence of an adenovirus vector expressing a modified CEA antigen), such as positions 1057 to 3165 of SEQ ID NO: 40 or full-length SEQ ID NO: 40.

### VII. Prostate Cancer

Disclosed herein include methods for treating prostate cancer comprising administering to a subject in need thereof compositions comprising replication-defective vectors comprising one or more nucleic acid sequences encoding PSA family antigen (e.g., PSA and/or PSMA), and/or one or more nucleic acid sequences encoding mucin family antigen such as MUC1, and/or one or more nucleic acid sequences encoding Brachyury, and/or one or more nucleic acid sequences encoding CEA in same or separate replication-defective vectors.

Prostate cancer, also known as carcinoma of the prostate, is the development of cancer in the prostate, a gland in the male reproductive system. Most prostate cancers are slow growing; however, some grow relatively quickly. The cancer cells may spread from the prostate to other parts of the body, particularly the bones and lymph nodes. It may initially cause no symptoms. In later stages it can lead to difficulty urinating, blood in the urine, or pain in the pelvis, back or when urinating. A disease known as benign prostatic hyperplasia may produce similar symptoms. Other late symptoms may include feeling tired due to low levels of red blood cells.

Early prostate cancer usually has no clear symptoms. Sometimes, however, prostate cancer does cause symptoms, often similar to those of diseases such as benign prostatic hyperplasia. These include frequent urination, nocturia (increased urination at night), difficulty starting and maintaining a steady stream of urine, hematuria (blood in the urine), and dysuria (painful urination). A study based on the 1998 Patient Care Evaluation in the US found that about a third of patients diagnosed with prostate cancer had one or more such symptoms, while two thirds had no symptoms.

Prostate cancer is associated with urinary dysfunction as the prostate gland surrounds the prostatic urethra. Changes within the gland, therefore, directly affect urinary function. Because the vas deferens deposits seminal fluid into the prostatic urethra, and secretions from the prostate gland itself are included in semen content, prostate cancer may also cause problems with sexual function and performance, such as difficulty achieving erection or painful ejaculation.

In certain aspects, advanced prostate cancer can spread to other parts of the body, possibly causing additional symptoms. The most common symptom is bone pain, often in the vertebrae (bones of the spine), pelvis, or ribs. The spread of cancer into other bones, such as the femur, is usually a result of spreading to the proximal or nearby part of the bone. Prostate cancer in the spine can also compress the spinal cord, causing tingling, leg weakness and urinary and fecal incontinence.

Prostate cancer is an ideal candidate for immunotherapy for several reasons. The slow growing nature of cancer within the prostate allows sufficient time to generate an anti-tumor immune response following a prime/boost or multiple immunization strategies. In addition, prostate cancer expresses numerous tumor associated antigens (TAAs) that include the Prostate Specific Antigen (PSA), Prostatic Acid Phosphatase (PAP), Prostate Specific Membrane Antigen (PSMA), Prostate Stem Cell Antigen (PSCA) and Six Transmembrane Epithelial Antigen of the Prostate (STEAP). All of these TAAs provide multiple potential immunological anti-tumor targets and the ideal combination of antigens to target has yet to be fully determined.

The presence of PSA in patient serum enables the malignancy to be detected early and, in some cases, before tumors are radiologically detectable. This in turn can facilitate earlier treatment. Circulating T cells that react with prostate TAAs have previously been detected, which suggests that self-tolerance to these antigens can be overcome. The prostate is considered to be a non-essential organ and therefore induced immunological responses directed against specific prostate TAAs should not cause acute off target toxicity. Most importantly, the first prostate cancer specific immunotherapy, Sipuleucel-T (Provenge®, Dendreon Corporation, Seattle, Wash.), was licensed by the US Food and Drug Administration (FDA) in 2010 for asymptomatic or minimally symptomatic castrate resistant prostate cancer (CRPC). Sipuleucel-T consists of autologous peripheral blood mononuclear cells with antigen presenting dendritic cells that have been activated ex vivo with a recombinant fusion protein (PA2024) consisting of PAP linked to granulocyte-macrophage colony stimulating factor (GM-CSF). In a phase III trial, CPRC patients receiving Sipuleucel-T exhibited a 22% reduction in mortality. The success of the therapeutic Sipuleucel-T has now paved the way for other immunotherapeutic prostate cancer vaccines to be granted regulatory approval and enter the market.

A poxviral PSA based vaccine (Vaccinia-PSA prime, Fowlpox-PSA boost) was evaluated in a randomized phase 2 trial. Subjects with minimally symptomatic metastatic castration resistant Prostate cancer were randomized 2/1 (85/41) to vaccine therapy versus placebo. Treated subjects had prolonged median overall survival (OS) 26 vs. 18 months. This approach has been taken to pivotal randomized phase 3 trial, now fully enrolled (N=1200), and awaiting event driven results.

In addition, an Adenoviral-PSA approach is being developed. PSA has been incorporated into a replication incompetent early generation Ad5 [E1−]-based vector platform and tested in a phase 1 trial. Sequential cohorts of subjects had increasing doses of a single injection of Ad5-PSA. Most subjects developed detectable cellular mediated anti-PSA responses 18/32 (67%). This approach is being evaluated in a phase 2 trial using multiple doses (3 injections) at 1 month intervals.

Thus, PSA based vaccination approaches have preliminary evidence of clinical activity as well as an ability to induce anti-PSA directed cellular immunity. Improved vectors, such as the new Etubics Ad5 [E1−, E2b−]-based vector platform described herein, should facilitate clinical development of this targeted approach. Non-replicating adenoviral vectors should improve the safety of this approach, and the ability to circumvent neutralizing anti-viral immune responses would enable sustained boosting to maximize immune responses. These features can be provided by the Ad5 [E1−, E2b−] vectors as described herein.

Standard treatment of aggressive prostate cancers may involve surgery (i.e., radical prostatectomy), radiation therapy including brachytherapy (prostate brachytherapy) and external beam radiation therapy, high-intensity focused ultrasound (HIFU), chemotherapy, oral chemotherapeutic drugs (Temozolomide/TMZ), cryosurgery, hormonal therapy, or some combination.

In certain embodiments, the Ad5 [E1−, E2b−]-PSA- and/or -PSMA-based vaccination approaches as used herein can be combined with any available prostate cancer therapy, such as the examples described above.

### VIII. Vectors

Certain aspects include transferring into a cell an expression construct comprising one or more nucleic acid sequences encoding one or more target antigens such as PSA, MUC1, Brachyury, PSMA, CEA, or a combination thereof. In certain embodiments, transfer of an expression construct into a cell may be accomplished using a viral vector. A viral vector may be used to include those constructs containing viral sequences sufficient to express a recombinant gene construct that has been cloned therein.

In particular embodiments, the viral vector is an adenovirus vector. Adenoviruses are a family of DNA viruses characterized by an icosahedral, non-enveloped capsid containing a linear double-stranded genome. Of the human adenoviruses, none are associated with any neoplastic disease, and only cause relatively mild, self-limiting illness in immunocompetent individuals.

Adenovirus vectors may have low capacity for integration into genomic DNA. Adenovirus vectors may result in highly efficient gene transfer. Additional advantages of adenovirus vectors include that they are efficient at gene delivery to both nondividing and dividing cells and can be produced in large quantities.

In contrast to integrating viruses, the adenoviral infection of host cells may not result in chromosomal integration because adenoviral DNA can replicate in an episomal manner without potential genotoxicity. Also, adenovirus vectors may be structurally stable, and no genome rearrangement has been detected after extensive amplification. Adenovirus is particularly suitable for use as a gene transfer vector because of its mid-sized genome, ease of manipulation, high titer, wide target-cell range and high infectivity.

The first genes expressed by the virus are the E1 genes, which act to initiate high-level gene expression from the other Ad5 gene promoters present in the wild type genome. Viral DNA replication and assembly of progeny virions occur within the nucleus of infected cells, and the entire life cycle takes about 36 hr with an output of approximately 10⁴ virions per cell.

The wild type Ad5 genome is approximately 36 kb, and encodes genes that are divided into early and late viral functions, depending on whether they are expressed before or after DNA replication. The early/late delineation is nearly absolute, since it has been demonstrated that super-infection of cells previously infected with an Ad5 results in lack of late gene expression from the super-infecting virus until after it has replicated its own genome. Without being bound by theory, this is likely due to a replication dependent cis-activation of the Ad5 major late promoter (MLP), preventing late gene expression (primarily the Ad5 capsid proteins) until replicated genomes are present to be encapsulated. The composition and methods may take advantage of these features in the development of advanced generation Ad vectors/vaccines.

The adenovirus vector may be replication defective, or at least conditionally defective. The adenovirus may be of any of the 42 different known serotypes or subgroups A-F and other serotypes or subgroups are envisioned. Adenovirus type 5 of subgroup C may be used in particular embodiments in order to obtain a replication-defective adenovirus vector. This is because Adenovirus type 5 is a human adenovirus about which a great deal of biochemical and genetic information is known, and it has historically been used for most constructions employing adenovirus as a vector.

Adenovirus growth and manipulation is known to those of skill in the art, and exhibits broad host range in vitro and in vivo. Modified viruses, such as adenoviruses with alteration of the CAR domain, may also be used. Methods for enhancing delivery or evading an immune response, such as liposome encapsulation of the virus, are also envisioned.

The vector may comprise a genetically engineered form of adenovirus, such as an E2 deleted adenoviral vector, or more specifically, an E2b deleted adenoviral vector. The term “E2b deleted,” as used herein, refers to a specific DNA sequence that is mutated in such a way so as to prevent expression and/or function of at least one E2b gene product. Thus, in certain embodiments, “E2b deleted” refers to a specific DNA sequence that is deleted (removed) from the Ad genome. E2b deleted or “containing a deletion within the E2b region” refers to a deletion of at least one base pair within the E2b region of the Ad genome. In certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may be a deletion that prevents expression and/or function of at least one E2b gene product and therefore, encompasses deletions within exons encoding portions of E2b-specific proteins as well as deletions within promoter and leader sequences. In certain embodiments, an E2b deletion is a deletion that prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As would be understood by the skilled artisan upon reading the present disclosure, other regions of the Ad genome can be deleted. Thus to be “deleted” in a particular region of the Ad genome, as used herein, refers to a specific DNA sequence that is mutated in such a way so as to prevent expression and/or function of at least one gene product encoded by that region. In certain embodiments, to be “deleted” in a particular region refers to a specific DNA sequence that is deleted (removed) from the Ad genome in such a way so as to prevent the expression and/or the function encoded by that region (e.g., E2b functions of DNA polymerase or preterminal protein function). “Deleted” or “containing a deletion” within a particular region refers to a deletion of at least one base pair within that region of the Ad genome.

Thus, in certain embodiments, more than one base pair is deleted and in further embodiments, at least 20, 30, 40, 50, 60, 70, 80, 90, 100, 110, 120, 130, 140, or 150 base pairs are deleted from a particular region. In another embodiment, the deletion is more than 150, 160, 170, 180, 190, 200, 250, or 300 base pairs within a particular region of the Ad genome. These deletions are such that expression and/or function of the gene product encoded by the region is prevented. Thus deletions encompass deletions within exons encoding portions of proteins as well as deletions within promoter and leader sequences. In a further embodiment, “deleted” in a particular region of the Ad genome refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

In certain embodiments, the adenovirus vectors contemplated for use include E2b deleted adenovirus vectors that have a deletion in the E2b region of the Ad genome and, optionally, the E1 region. In some cases, such vectors do not have any other regions of the Ad genome deleted.

In another embodiment, the adenovirus vectors contemplated for use include E2b deleted adenovirus vectors that have a deletion in the E2b region of the Ad genome and, optionally, deletions in the E1 and E3 regions. In some cases, such vectors have no other regions deleted.

In a further embodiment, the adenovirus vectors contemplated for use include adenovirus vectors that have a deletion in the E2b region of the Ad genome and, optionally, deletions in the E1, E3, and also optionally, partial or complete removal of the E4 regions. In some cases, such vectors have no other deletions.

In another embodiment, the adenovirus vectors contemplated for use include adenovirus vectors that have a deletion in the E2b region of the Ad genome and, optionally, deletions in the E1 and/or E4 regions. In some cases, such vectors contain no other deletions.

In an additional embodiment, the adenovirus vectors contemplated for use include adenovirus vectors that have a deletion in the E2a, E2b, and/or E4 regions of the Ad genome. In some cases, such vectors have no other deletions.

In one embodiment, the adenovirus vectors for use herein comprise vectors having the E1 and/or DNA polymerase functions of the E2b region deleted. In some cases, such vectors have no other deletions.

In a further embodiment, the adenovirus vectors for use herein have the E1 and/or the preterminal protein functions of the E2b region deleted. In some cases, such vectors have no other deletions.

In another embodiment, the adenovirus vectors for use herein have the E1, DNA polymerase and/or the preterminal protein functions deleted. In some cases, such vectors have no other deletions. In one particular embodiment, the adenovirus vectors contemplated for use herein are deleted for at least a portion of the E2b region and/or the E1 region.

In some cases, such vectors are not “gutted” adenovirus vectors. In this regard, the vectors may be deleted for both the DNA polymerase and the preterminal protein functions of the E2b region. In an additional embodiment, the adenovirus vectors for use include adenovirus vectors that have a deletion in the E1, E2b, and/or 100K regions of the adenovirus genome. In certain embodiments, the adenovirus vector may be a “gutted” adenovirus vector.

In one embodiment, the adenovirus vectors for use herein comprise vectors having the E1, E2b, and/or protease functions deleted. In some cases, such vectors have no other deletions.

In a further embodiment, the adenovirus vectors for use herein have the E1 and/or the E2b regions deleted, while the fiber genes have been modified by mutation or other alterations (e.g., to alter Ad tropism). Removal of genes from the E3 or E4 regions may be added to any of the mentioned adenovirus vectors.

The deleted adenovirus vectors can be generated using recombinant techniques known in the art (see e.g., Amalfitano, et al. J. Virol. 1998; 72:926-33; Hodges, et al. J Gene Med 2000; 2:250-59). As would be recognized by a skilled artisan, the adenovirus vectors for use in certain aspects can be successfully grown to high titers using an appropriate packaging cell line that constitutively expresses E2b gene products and products of any of the necessary genes that may have been deleted. In certain embodiments, HEK-293-derived cells that not only constitutively express the E1 and DNA polymerase proteins, but also the Ad-preterminal protein, can be used. In one embodiment, E.C7 cells are used to successfully grow high titer stocks of the adenovirus vectors (see e.g., Amalfitano, et al. J. Virol. 1998; 72:926-33; Hodges, et al. J Gene Med 2000; 2:250-59)

In order to delete critical genes from self-propagating adenovirus vectors, the proteins encoded by the targeted genes may be coexpressed in HEK-293 cells, or similar, along with the E1 proteins. Therefore, only those proteins which are non-toxic when coexpressed constitutively (or toxic proteins inducibly-expressed) can be utilized. Coexpression in HEK-293 cells of the E1 and E4 genes has been demonstrated (utilizing inducible, not constitutive, promoters) (Yeh, et al. J. Virol. 1996; 70:559; Wang et al. Gene Therapy 1995; 2:775; and Gorziglia, et al. J. Virol. 1996; 70:4173). The E1 and protein IX genes (a virion structural protein) have been coexpressed (Caravokyri, et al. J. Virol. 1995; 69: 6627), and coexpression of the E1, E4, and protein IX genes has also been described (Krougliak, et al. Hum. Gene Ther. 1995; 6:1575). The E1 and 100k genes have been successfully expressed in transcomplementing cell lines, as have E1 and protease genes (Oualikene, et al. Hum Gene Ther 2000; 11:1341-53; Hodges, et al. J. Virol 2001; 75:5913-20).

Cell lines coexpressing E1 and E2b gene products for use in growing high titers of E2b deleted Ad particles are described in U.S. Pat. No. 6,063,622. The E2b region encodes the viral replication proteins which are absolutely required for Ad genome replication (Doerfler, et al. Chromosoma 1992; 102:S39-S45). Useful cell lines constitutively express the approximately 140 kDa Ad-DNA polymerase and/or the approximately 90 kDa preterminal protein. In particular, cell lines that have high-level, constitutive coexpression of the E1, DNA polymerase, and preterminal proteins, without toxicity (e.g., E.C7), are desirable for use in propagating Ad for use in multiple vaccinations. These cell lines permit the propagation of adenovirus vectors deleted for the E1, DNA polymerase, and preterminal proteins.

The recombinant adenovirus vector can be propagated using techniques available in the art. For example, in certain embodiments, tissue culture plates containing E.C7 cells are infected with the adenovirus vector virus stocks at an appropriate MOI (e.g., 5) and incubated at 37.0° C. for 40-96 hrs. The infected cells are harvested, resuspended in 10 mM Tris-CI (pH 8.0), and sonicated, and the virus is purified by two rounds of cesium chloride density centrifugation. In certain techniques, the virus containing band is desalted over a Sephadex CL-6B column (Pharmacia Biotech, Piscataway, N.J.), sucrose or glycerol is added, and aliquots are stored at −80° C. In some embodiments, the virus vector is placed in a solution designed to enhance its stability, such as A195 (Evans, et al. J Pharm Sci 2004; 93:2458-75). The titer of the stock is measured (e.g., by measurement of the optical density at 260 nm of an aliquot of the virus after SDS lysis). In another embodiment, plasmid DNA, either linear or circular, encompassing the entire recombinant E2b deleted adenovirus vector can be transfected into E.C7, or similar cells, and incubated at 37.0° C. until evidence of viral production is present (e.g., the cytopathic effect). The conditioned media from these cells can then be used to infect more E.C7, or similar cells, to expand the amount of virus produced, before purification. Purification can be accomplished by two rounds of cesium chloride density centrifugation or selective filtration. In certain embodiments, the virus may be purified by column chromatography, using commercially available products (e.g., Adenopure from Puresyn, Inc., Malvem, Pa.) or custom made chromatographic columns.

In certain embodiments, the recombinant adenovirus vector may comprise enough of the virus to ensure that the cells to be infected are confronted with a certain number of viruses. Thus, there may be provided a stock of recombinant Ad, particularly an RCA-free stock of recombinant Ad. The preparation and analysis of Ad stocks can use any methods available in the art. Viral stocks vary considerably in titer, depending largely on viral genotype and the protocol and cell lines used to prepare them. The viral stocks can have a titer of at least about 10⁶, 10⁷, or 10⁸ virus particles (VPs)/ml, and many such stocks can have higher titers, such as at least about 10⁹, 10¹⁰, 10″, or 10¹² VPs/ml.

Certain aspects contemplate the use of E2b deleted adenovirus vectors, such as those described in U.S. Pat. Nos. 6,063,622; 6,451,596; 6,057,158; 6,083,750; and 8,298,549. The vectors with deletions in the E2b regions in many cases cripple viral protein expression and/or decrease the frequency of generating replication competent Ad (RCA).

Propagation of these E2b deleted adenovirus vectors can be done utilizing cell lines that express the deleted E2b gene products. Certain aspects also provide such packaging cell lines; for example E.C7 (formally called C-7), derived from the HEK-293 cell line.

In further aspects, the E2b gene products, DNA polymerase and preterminal protein, can be constitutively expressed in E.C7, or similar cells along with the E1 gene products. Transfer of gene segments from the Ad genome to the production cell line has immediate benefits: (1) increased carrying capacity; and, (2) a decreased potential of RCA generation, typically requiring two or more independent recombination events to generate RCA. The E1, Ad DNA polymerase and/or preterminal protein expressing cell lines used herein can enable the propagation of adenovirus vectors with a carrying capacity approaching 13 kb, without the need for a contaminating helper virus. In addition, when genes critical to the viral life cycle are deleted (e.g., the E2b genes), a further crippling of Ad to replicate or express other viral gene proteins occurs. This can decrease immune recognition of virally infected cells, and allow for extended durations of foreign transgene expression.

E1, DNA polymerase, and preterminal protein deleted vectors are typically unable to express the respective proteins from the E1 and E2b regions. Further, they may show a lack of expression of most of the viral structural proteins. For example, the major late promoter (MLP) of Ad is responsible for transcription of the late structural proteins L1 through L5. Though the MLP is minimally active prior to Ad genome replication, the highly toxic Ad late genes are primarily transcribed and translated from the MLP only after viral genome replication has occurred. This cis-dependent activation of late gene transcription is a feature of DNA viruses in general, such as in the growth of polyoma and SV-40. The DNA polymerase and preterminal proteins are important for Ad replication (unlike the E4 or protein IX proteins). Their deletion can be extremely detrimental to adenovirus vector late gene expression, and the toxic effects of that expression in cells such as APCs.E1-deleted adenovirus vectors

Certain aspects contemplate the use of E1-deleted adenovirus vectors. First generation, or E1-deleted adenovirus vectors Ad5 [E1−] are constructed such that a transgene replaces only the E1 region of genes. Typically, about 90% of the wild-type Ad5 genome is retained in the vector. Ad5 [E1−] vectors have a decreased ability to replicate and cannot produce infectious virus after infection of cells not expressing the Ad5 E1 genes. The recombinant Ad5 [E1−] vectors are propagated in human cells (typically 293 cells) allowing for Ad5 [E1−] vector replication and packaging. Ad5 [E1−] vectors have a number of positive attributes; one of the most important is their relative ease for scale up and cGMP production. Currently, well over 220 human clinical trials utilize Ad5 [E1−] vectors, with more than two thousand subjects given the virus subcutaneously, intramuscularly, or intravenously.

Additionally, Ad5 vectors do not integrate; their genomes remain episomal. Generally, for vectors that do not integrate into the host genome, the risk for insertional mutagenesis and/or germ-line transmission is extremely low if at all. Conventional Ad5 [E1−] vectors have a carrying capacity that approaches 7 kb.

Studies in humans and animals have demonstrated that pre-existing immunity against Ad5 can be an inhibitory factor to commercial use of Ad-based vaccines. The preponderance of humans have antibody against Ad5, the most widely used subtype for human vaccines, with two-thirds of humans studied having lympho-proliferative responses against Ad5. This pre-existing immunity can inhibit immunization or re-immunization using typical Ad5 vaccines and may preclude the immunization of a vaccine against a second antigen, using an Ad5 vector, at a later time. Overcoming the problem of pre-existing anti-vector immunity has been a subject of intense investigation. Investigations using alternative human (non-Ad5 based) Ad5 subtypes or even non-human forms of Ad5 have been examined. Even if these approaches succeed in an initial immunization, subsequent vaccinations may be problematic due to immune responses to the novel Ad5 subtype.

To avoid the Ad5 immunization barrier, and improve upon the limited efficacy of first generation Ad5 [E1−] vectors to induce optimal immune responses, there are provided certain embodiments related to a next generation Ad5 vector based vaccine platform. The next generation Ad5 platform has additional deletions in the E2b region, removing the DNA polymerase and the preterminal protein genes. The Ad5 [E1−, E2b−] platform has an expanded cloning capacity that is sufficient to allow inclusion of many possible genes. Ad5 [E1−, E2b−] vectors have up to about 12 kb gene-carrying capacity as compared to the 7 kb capacity of Ad5 [E1−] vectors, providing space for multiple genes if needed. In some embodiments, an insert of more than 1, 2, 3, 4, 5, 6, 7, 8, 9, 10, or 11 kb is introduced into an Ad5 vector, such as the Ad5 [E1−, E2b−] vector.

Deletion of the E2b region may confer advantageous immune properties on the Ad5 vectors, often eliciting potent immune responses to target antigens, such as PSA, PSMA, MUC1, Brachyury, CEA, or a combination thereof, while minimizing the immune responses to Ad viral proteins.

In various embodiments, Ad5 [E1−, E2b−] vectors may induce a potent CMI, as well as antibodies against the vector expressed target antigens, such as PSA, PSMA, MUC1, Brachyury, CEA, or a combination thereof, even in the presence of Ad immunity.

Ad5 [E1−, E2b−] vectors also have reduced adverse reactions as compared to Ad5 [E1−] vectors, in particular the appearance of hepatotoxicity and tissue damage.

Certain aspects of these Ad5 vectors are that expression of Ad late genes is greatly reduced. For example, production of the capsid fiber proteins could be detected in vivo for Ad5 [E1−] vectors, while fiber expression was ablated from Ad5 [E1−, E2b−] vector vaccines. The innate immune response to wild type Ad is complex. Proteins deleted from the Ad5 [E1−, E2b−] vectors generally play an important role. Specifically, Ad5 [E1−, E2b−] vectors with deletions of preterminal protein or DNA polymerase display reduced inflammation during the first 24 to 72 hours following injection compared to Ad5 [E1−] vectors. In various embodiments, the lack of Ad5 gene expression renders infected cells invisible to anti-Ad activity and permits infected cells to express the transgene for extended periods of time, which develops immunity to the target.

Various embodiments contemplate increasing the capability for the Ad5 [E1−, E2b−] vectors to transduce dendritic cells, improving antigen specific immune responses in the vaccine by taking advantage of the reduced inflammatory response against Ad5 [E1−, E2b−] vector viral proteins and the resulting evasion of pre-existing Ad immunity.

In some cases, this immune induction may take months. Ad5 [E1−, E2b−] vectors not only are safer than, but appear to be superior to Ad5 [E1−] vectors in regard to induction of antigen specific immune responses, making them much better suitable as a platform to deliver tumor vaccines that can result in a clinical response.

In certain embodiments, methods and compositions are provided by taking advantage of an Ad5 [E1−, E2b−] vector system for developing a therapeutic tumor vaccine that overcomes barriers found with other Ad5 systems and permits the immunization of people who have previously been exposed to Ad5.

E2b deleted vectors may have up to a 13 kb gene-carrying capacity as compared to the 5 to 6 kb capacity of First Generation adenovirus vectors, easily providing space for nucleic acid sequences encoding any of a variety of target antigens, such as PSA, PSMA, MUC1, Brachyury, or a combination thereof.

The E2b deleted adenovirus vectors also have reduced adverse reactions as compared to First Generation adenovirus vectors. E2b deleted vectors have reduced expression of viral genes, and this characteristic leads to extended transgene expression in vivo.

Compared to first generation adenovirus vectors, certain embodiments of the Second Generation E2b deleted adenovirus vectors contain additional deletions in the DNA polymerase gene (pol) and deletions of the pre-terminal protein (pTP).

It appears that Ad proteins expressed from adenovirus vectors play an important role. Specifically, the deletions of pre-terminal protein and DNA polymerase in the E2b deleted vectors appear to reduce inflammation during the first 24 to 72 hours following injection, whereas First Generation adenovirus vectors stimulate inflammation during this period.

In addition, it has been reported that the additional replication block created by E2b deletion also leads to a 10,000 fold reduction in expression of Ad late genes, well beyond that afforded by E1, E3 deletions alone. The decreased levels of Ad proteins produced by E2b deleted adenovirus vectors effectively reduce the potential for competitive, undesired, immune responses to Ad antigens, responses that prevent repeated use of the platform in Ad immunized or exposed individuals.

The reduced induction of inflammatory response by second generation E2b deleted vectors results in increased potential for the vectors to express desired vaccine antigens, such as PSA, PSMA, MUC1, Brachyury, CEA, or a combination thereof, during the infection of antigen presenting cells (i.e., dendritic cells), decreasing the potential for antigenic competition, resulting in greater immunization of the vaccine to the desired antigen relative to identical attempts with First Generation adenovirus vectors.

E2b deleted adenovirus vectors provide an improved Ad-based vaccine candidate that is safer, more effective, and more versatile than previously described vaccine candidates using First Generation adenovirus vectors.

Thus, first generation, E1-deleted Adenovirus subtype 5 (Ad5)-based vectors, although promising platforms for use as vaccines, may be impeded in activity by naturally occurring or induced Ad-specific neutralizing antibodies.

Without being bound by theory, Ad5-based vectors with deletions of the E1 and the E2b regions (Ad5 [E1−, E2b−]), the latter encoding the DNA polymerase and the pre-terminal protein, for example by virtue of diminished late phase viral protein expression, may avoid immunological clearance and induce more potent immune responses against the encoded antigen transgene, such as PSA, PSMA, MUC1, Brachyury, CEA, or a combination thereof, in Ad-immune hosts.

### IX. Heterologous Nucleic Acids

In some embodiments, vectors, such as adenovirus vectors, may comprise heterologous nucleic acid sequences that encode one or more tumor antigens such as PSA, PSMA, MUC1, Brachyury, CEA, or a combination thereof, fusions thereof or fragments thereof, which can modulate the immune response. In certain aspects, there may be provided a Second Generation E2b deleted adenovirus vectors that comprise a heterologous nucleic acid sequence encoding one or more tumor antigens such as PSA, PSMA, MUC1, Brachyury, CEA, or a combination thereof.

As such, there may be provided polynucleotides that encode PSA, PSMA, MUC1, Brachyury, CEA, or a combination thereof from any source as described further herein, vectors or constructs comprising such polynucleotides and host cells transformed or transfected with such vectors or expression constructs.

The terms “nucleic acid” and “polynucleotide” are used essentially interchangeably herein. As will be also recognized by the skilled artisan, polynucleotides used herein may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide as disclosed herein, and a polynucleotide may, but need not, be linked to other molecules and/or support materials. An isolated polynucleotide, as used herein, means that a polynucleotide is substantially away from other coding sequences. For example, an isolated DNA molecule as used herein does not contain large portions of unrelated coding DNA, such as large chromosomal fragments or other functional genes or polypeptide coding regions. Of course, this refers to the DNA molecule as originally isolated, and does not exclude genes or coding regions later added to the segment through recombination in the laboratory.

As will be understood by those skilled in the art, the polynucleotides can include genomic sequences, extra-genomic and plasmid-encoded sequences and smaller engineered gene segments that express, or may be adapted to express target antigens as described herein, fragments of antigens, peptides and the like. Such segments may be naturally isolated, or modified synthetically by the hand of man.

Polynucleotides may comprise a native sequence (i.e., an endogenous sequence that encodes one or more tumor antigens such as PSA, PSMA, MUC1, Brachyury, CEA, or a combination thereof or a portion thereof) or may comprise a sequence that encodes a variant or derivative of such a sequence. In certain embodiments, the polynucleotide sequences set forth herein encode one or more mutated tumor antigens such as PSA, PSMA, MUC1, Brachyury, CEA, or a combination thereof. In some embodiments, polynucleotides represent a novel gene sequence that has been optimized for expression in specific cell types (i.e., human cell lines) that may substantially vary from the native nucleotide sequence or variant but encode a similar protein antigen.

In other related embodiments, there may be provided polynucleotide variants having substantial identity to native sequences encoding one or more tumor antigens such as PSA, PSMA, MUC1, Brachyury, CEA, or a combination thereof, for example those comprising at least 70, 80, 90, 95, 96, 97, 98, or 99% sequence identity or any derivable range or value thereof, particularly at least 75% up to 99% or higher, sequence identity compared to a native polynucleotide sequence encoding one or more tumor antigens such as PSA, MUC1, Brachyury, CEA, or a combination thereof using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In some embodiments, polynucleotide variants contain one or more substitutions, additions, deletions and/or insertions, particularly such that the immunogenicity of the epitope of the polypeptide encoded by the variant polynucleotide or such that the immunogenicity of the heterologous target protein is not substantially diminished relative to a polypeptide encoded by the native polynucleotide sequence. As described elsewhere herein, the polynucleotide variants preferably encode a variant of one or more tumor antigens such as PSA, PSMA, MUC1, Brachyury, CEA, or a combination thereof, or a fragment (e.g., an epitope) thereof wherein the propensity of the variant polypeptide or fragment (e.g., epitope) thereof to react with antigen-specific antisera and/or T-cell lines or clones is not substantially diminished relative to the native polypeptide. The term “variants” should also be understood to encompass homologous genes of xenogenic origin.

In certain aspects, there may be provided polynucleotides that comprise or consist of at least about 5 up to a 1000 or more contiguous nucleotides encoding a polypeptide, including target protein antigens, as described herein, as well as all intermediate lengths there between. It will be readily understood that “intermediate lengths,” in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described herein may be extended at one or both ends by additional nucleotides not found in the native sequence encoding a polypeptide as described herein, such as an epitope or heterologous target protein. This additional sequence may consist of 1 up 20 nucleotides or more, at either end of the disclosed sequence or at both ends of the disclosed sequence.

The polynucleotides or fragments thereof, regardless of the length of the coding sequence itself, may be combined with other DNA sequences, such as promoters, expression control sequences, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 1000, 2000, 3000, 4000, 5000, 6000, 7000, 8000, 9000, 10,000, about 500, about 200, about 100, about 50 base pairs in length, and the like, (including all intermediate lengths) are contemplated to be useful in certain aspects.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff M O (1978) A model of evolutionary change in proteins—Matrices for detecting distant relationships. In Dayhoff M O (ed.) Atlas of Protein Sequence and Structure, National Biomedical Research Foundation, Washington D.C. Vol. 5, Suppl. 3, pp. 345-358; Hein J Unified Approach to Alignment and Phylogenes, pp. 626-645 (1990); Methods in Enzymology vol. 183, Academic Press, Inc., San Diego, Calif.; Higgins, et al. PM CABIOS 1989; 5:151-53; Myers E W, et al. CABIOS 1988; 4:11-17; Robinson E D Comb. Theor 1971; 11A 05; Saitou N, et al. Mol. Biol. Evol. 1987; 4:406-25; Sneath P H A and Sokal R R Numerical Taxonomy—the Principles and Practice of Numerical Taxonomy, Freeman Press, San Francisco, Calif. (1973); Wilbur W J, et al. Proc. Natl. Acad., Sci. USA 1983 80:726-30).

Alternatively, optimal alignment of sequences for comparison may be conducted by the local identity algorithm of Smith, et al. Add. APL. Math 1981; 2:482, by the identity alignment algorithm of Needleman, et al. Mol. Biol. 1970 48:443, by the search for similarity methods of Pearson and Lipman, Proc. Natl. Acad. Sci. USA 1988; 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al., Nucl. Acids Res. 1977 25:3389-3402, and Altschul et al. J. MoI. Biol. 1990 215:403-10, respectively. BLAST and BLAST 2.0 can be used, for example with the parameters described herein, to determine percent sequence identity for the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff, et al. Proc. Natl. Acad. Sci. USA 1989; 89:10915) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a particular antigen of interest, or fragment thereof, as described herein. Some of these polynucleotides bear minimal homology to the nucleotide sequence of any native gene. Nonetheless, polynucleotides that vary due to differences in codon usage are specifically contemplated.

Further, alleles of the genes comprising the polynucleotide sequences provided herein may also be contemplated. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment, a mutagenesis approach, such as site-specific mutagenesis, is employed for the preparation of variants and/or derivatives of nucleic acid sequences encoding one or more tumor antigens such as PSA, PSMA, MUC1, Brachyury, CEA, or a combination thereof, or fragments thereof, as described herein. By this approach, specific modifications in a polypeptide sequence can be made through mutagenesis of the underlying polynucleotides that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

Polynucleotide segments or fragments encoding the polypeptides may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202, by introducing selected sequences into recombinant vectors for recombinant production, and by other recombinant DNA techniques generally known to those of skill in the art of molecular biology (see for example, Current Protocols in Molecular Biology, John Wiley and Sons, NY, N.Y.).

In order to express a desired tumor antigen such as PSA, PSMA, MUC1, Brachyury, CEA, or a combination thereof, polypeptide or fragment thereof, or fusion protein comprising any of the above, as described herein, the nucleotide sequences encoding the polypeptide, or functional equivalents, are inserted into an appropriate vector such as a replication-defective adenovirus vector as described herein using recombinant techniques known in the art. The appropriate vector contains the necessary elements for the transcription and translation of the inserted coding sequence and any desired linkers.

Methods that are available to those skilled in the art may be used to construct these vectors containing sequences encoding one or more tumor antigens such as PSA, MUC1, Brachyury, CEA, or a combination thereof and appropriate transcriptional and translational control elements. These methods include in vitro recombinant DNA techniques, synthetic techniques, and in vivo genetic recombination. Such techniques are described, for example, in Amalfitano, et al. J. Virol. 1998; 72:926-33; Hodges, et al. J Gene Med 2000; 2:250-259; Sambrook J, et al. (1989) Molecular Cloning, A Laboratory Manual, Cold Spring Harbor Press, Plainview, N.Y., and Ausubel F M, et al. (1989) Current Protocols in Molecular Biology, John Wiley & Sons, New York. N.Y.

A variety of vector/host systems may be utilized to contain and produce polynucleotide sequences. These include, but are not limited to, microorganisms such as bacteria transformed with recombinant bacteriophage, plasmid, or cosmid DNA vectors; yeast transformed with yeast vectors; insect cell systems infected with virus vectors (e.g., baculovirus); plant cell systems transformed with virus vectors (e.g., cauliflower mosaic virus, CaMV; tobacco mosaic virus, TMV) or with bacterial vectors (e.g., Ti or pBR322 plasmids); or animal cell systems.

The “control elements” or “regulatory sequences” present in a vector, such as an adenovirus vector, are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, sequences encoding one or more tumor antigens such as PSA, PSMA, MUC1, Brachyury, CEA, or a combination thereof may be ligated into an Ad transcription/translation complex consisting of the late promoter and tripartite leader sequence. Insertion in a non-essential E1 or E3 region of the viral genome may be used to obtain a viable virus that is capable of expressing the polypeptide in infected host cells (Logan J, et al. Proc. Natl. Acad. Sci 1984; 87:3655-59). In addition, transcription enhancers, such as the Rous sarcoma virus (RSV) enhancer, may be used to increase expression in mammalian host cells.

Specific initiation signals may also be used to achieve more efficient translation of sequences encoding one or more tumor antigens such as PSA, PSMA, MUC1, Brachyury, CEA, or a combination thereof. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system which is used, such as those described in the literature (Scharf D., et al. Results Probl. Cell Differ. 1994; 20:125-62). Specific termination sequences, either for transcription or translation, may also be incorporated in order to achieve efficient translation of the sequence encoding the polypeptide of choice.

A variety of protocols for detecting and measuring the expression of polynucleotide-encoded products (e.g., one or more tumor antigens such as PSA, PSMA, MUC1, Brachyury, CEA, or a combination thereof), using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton R et al. (1990; Serological Methods, a Laboratory Manual, APS Press, St Paul. Minn.) and Maddox D E, et al. J. Exp. Med. 1983; 758:1211-16).

In certain embodiments, elements that increase the expression of the desired tumor antigens such as PSA, PSMA, MUC1, Brachyury, CEA, or a combination thereof may be incorporated into the nucleic acid sequence of expression constructs or vectors such as adenovirus vectors described herein. Such elements include internal ribosome binding sites (IRES; Wang, et al. Curr. Top. Microbiol. Immunol 1995; 203:99; Ehrenfeld, et al. Curr. Top. Microbiol. Immunol. 1995; 203:65; Rees, et al. Biotechniques 1996; 20:102; Sugimoto, et al. Biotechnology 1994; 2:694). IRES increase translation efficiency. As well, other sequences may enhance expression. For some genes, sequences especially at the 5′ end inhibit transcription and/or translation. These sequences are usually palindromes that can form hairpin structures. Any such sequences in the nucleic acid to be delivered are generally deleted. Expression levels of the transcript or translated product are assayed to confirm or ascertain which sequences affect expression. Transcript levels may be assayed by any known method, including Northern blot hybridization, RNase probe protection, and the like. Protein levels may be assayed by any known method, including ELISA.

As would be recognized by a skilled artisan, vectors, such as adenovirus vectors described herein, that comprise heterologous nucleic acid sequences can be generated using recombinant techniques known in the art, such as those described in Maione, et al. Proc Natl Acad Sci USA 2001; 98:5986-91; Maione, et al. Hum Gene Ther 2000 1:859-68; Sandig, et al. Proc Natl Acad Sci USA, 2000; 97:1002-07; Harui, et al. Gene Therapy 2004; 11:1617-26; Parks et al. Proc Natl Acad Sci USA 1996; 93:13565-570; DelloRusso, et al. Proc Natl Acad Sci USA 2002; 99:12979-984; Current Protocols in Molecular Biology, John Wiley and Sons, NY, N.Y.).

### X. Pharmaceutical Compositions

In certain aspects, there may be provided pharmaceutical compositions that comprise nucleic acid sequences encoding one or more one or more tumor antigens such as PSA, PSMA, MUC1, Brachyury, CEA, or a combination thereof against which an immune response is to be generated.

For example, the adenovirus vector stock described herein may be combined with an appropriate buffer, physiologically acceptable carrier, excipient, or the like. In certain embodiments, an appropriate number of adenovirus vector particles are administered in an appropriate buffer, such as, sterile PBS. In certain circumstances it will be desirable to deliver the adenovirus vector compositions disclosed herein parenterally, intravenously, intramuscularly, or even intraperitoneally.

In certain embodiments, solutions of the pharmaceutical compositions as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. In other embodiments, E2b deleted adenovirus vectors may be delivered in pill form, delivered by swallowing or by suppository.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria, molds and fungi.

The carrier can be a solvent or dispersion medium containing, for example, water, lipids, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be suitable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution may be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course suitably meet sterility, pyrogenicity, and the general safety and purity standards as required by FDA Office of Biology standards.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

Routes and frequency of administration of the therapeutic compositions described herein, as well as dosage, will vary from individual to individual, and from disease to disease, and may be readily established using standard techniques. In general, the pharmaceutical compositions and vaccines may be administered by injection (e.g., intracutaneous, intramuscular, intravenous or subcutaneous), intranasally (e.g., by aspiration), in pill form (e.g., swallowing, suppository for vaginal or rectal delivery). In certain embodiments, from 1 to 3 doses may be administered over a 6 week period and further booster vaccinations may be given periodically thereafter.

For example, a suitable dose is an amount of an adenovirus vector that, when administered as described above, is capable of promoting a target antigen immune response as described elsewhere herein. In certain embodiments, the immune response is at least 10-50% above the basal (i.e., untreated) level. Such response can be monitored by measuring the target antigen antibodies in a patient or by vaccine-dependent generation of cytolytic effector cells capable of killing target antigen-expressing cells in vitro, or other methods known in the art for monitoring immune responses. In certain aspects, the target antigens are PSA, PSMA, MUC1, Brachyury, CEA, or a combination thereof.

In general, an appropriate dosage and treatment regimen provides the adenovirus vectors in an amount sufficient to provide prophylactic benefit. Protective immune responses may generally be evaluated using standard proliferation, cytotoxicity or cytokine assays, which may be performed using samples obtained from a patient before and after immunization (vaccination).

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

While one advantage of compositions and methods described herein is the capability to administer multiple vaccinations with the same adenovirus vectors, particularly in individuals with preexisting immunity to Ad, the adenoviral vaccines described herein may also be administered as part of a prime and boost regimen. A mixed modality priming and booster inoculation scheme may result in an enhanced immune response. Thus, one aspect is a method of priming a subject with a plasmid vaccine, such as a plasmid vector comprising nucleic acid sequences encoding one or more tumor antigens such as PSA, PSMA, MUC1, Brachyury, CEA, or a combination thereof, by administering the plasmid vaccine at least one time, allowing a predetermined length of time to pass, and then boosting by administering the adenovirus vector described herein.

Multiple primings, e.g., 1-3, may be employed, although more may be used. The length of time between priming and boost may typically vary from about six months to a year, but other time frames may be used.

In certain embodiments, pharmaceutical compositions may comprise, for example, at least about 0.1% of therapeutic agents, such as the expression constructs or vectors used herein as vaccine, a related lipid nanovesicle, or an exosome or nanovesicle loaded with therapeutic agents. In other embodiments, the therapeutic agent may comprise from about 2% to about 75% of the weight of the unit, or from about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

In certain aspects, compositions comprising a vaccination regime as described herein can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. The compositions described throughout can be formulated into a pharmaceutical medicament and be used to treat a human or mammal, in need thereof, diagnosed with a disease, e.g., cancer, or to enhances an immune response.

In certain embodiments, the viral vectors or compositions described herein may be administered in conjunction with one or more immunostimulants, such as an adjuvant. An immunostimulant refers to essentially any substance that enhances or potentiates an immune response (antibody and/or cell-mediated) to an antigen. One type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories); Merck Adjuvant 65 (Merck and Company, Inc.) AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, IFN-γ, TNFα, IL-2, IL-8, IL-12, IL-18, IL-7, IL-3, IL-4, IL-5, IL-6, IL-9, IL-10, IL-13, IL-15, IL-16, IL-23, and/or IL-32, and others, like growth factors, may also be used as adjuvants.

Within certain embodiments, the adjuvant composition can be one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient may support an immune response that includes Th1- and/or Th2-type responses. Within certain embodiments, in which a response is predominantly Th1-type, the level of Th1-type cytokines will increase to a greater extent than the level of Th2-type cytokines. The levels of these cytokines may be readily assessed using standard assays. Thus, various embodiments relate to therapies raising an immune response against a target antigen, for example PSA, PSMA, MUC1, Brachyury, CEA, or a combination thereof, using cytokines, e.g., IFN-γ, TNFα, IL-2, IL-8, IL-12, IL-18, IL-7, IL-3, IL-4, IL-5, IL-6, IL-9, IL-10, IL-13 and/or IL-15 supplied concurrently with a replication defective viral vector treatment. In some embodiments, a cytokine or a nucleic acid encoding a cytokine, is administered together with a replication defective viral described herein. In some embodiments, cytokine administration is performed prior or subsequent to viral vector administration. In some embodiments, a replication defective viral vector capable of raising an immune response against a target antigen, for example PSA, PSMA, MUC1, Brachyury, CEA, or a combination thereof, further comprises a sequence encoding a cytokine.

Certain illustrative adjuvants for eliciting a predominantly Th1-type response include, for example, a combination of monophosphoryl lipid A, such as 3-de-O-acylated monophosphoryl lipid A, together with an aluminum salt. MPL® adjuvants are commercially available (see, e.g., U.S. Pat. Nos. 4,436,727; 4,877,611; 4,866,034; and 4,912,094). CpG-containing oligonucleotides (in which the CpG dinucleotide is unmethylated) also induce a predominantly Th1 response. (see, e.g., WO 96/02555, WO 99/33488 and U.S. Pat. Nos. 6,008,200 and 5,856,462). Immunostimulatory DNA sequences can also be used.

Another adjuvant for use in some embodiments comprises a saponin, such as Quil A, or derivatives thereof, including QS21 and QS7 (Aquila Biopharmaceuticals Inc.), Escin; Digitonin; or Gypsophila or Chenopodium quinoa saponins. Other formulations may include more than one saponin in the adjuvant combinations, e.g., combinations of at least two of the following group comprising QS21, QS7, Quil A, β-escin, or digitonin.

In some embodiments, the compositions may be delivered by intranasal sprays, inhalation, and/or other aerosol delivery vehicles. The delivery of drugs using intranasal microparticle resins and lysophosphatidyl-glycerol compounds can be employed (see, e.g., U.S. Pat. No. 5,725,871). Likewise, illustrative transmucosal drug delivery in the form of a polytetrafluoroetheylene support matrix can be employed (see, e.g., U.S. Pat. No. 5,780,045).

Liposomes, nanocapsules, microparticles, lipid particles, vesicles, and the like, can be used for the introduction of the compositions as described herein into suitable hot cells/organisms. Compositions as described herein may be formulated for delivery either encapsulated in a lipid particle, a liposome, a vesicle, a nanosphere, or a nanoparticle or the like. Alternatively, compositions as described herein can be bound, either covalently or non-covalently, to the surface of such carrier vehicles. Liposomes can be used effectively to introduce genes, various drugs, radiotherapeutic agents, enzymes, viruses, transcription factors, allosteric effectors and the like, into a variety of cultured cell lines and animals. Furthermore, the use of liposomes does not appear to be associated with autoimmune responses or unacceptable toxicity after systemic delivery. In some embodiments, liposomes are formed from phospholipids dispersed in an aqueous medium and spontaneously form multilamellar concentric bilayer vesicles (i.e., multilamellar vesicles (MLVs)).

In some embodiments, there are provided pharmaceutically-acceptable nanocapsule formulations of the compositions or vectors as described herein. Nanocapsules can generally entrap pharmaceutical compositions in a stable and reproducible way. To avoid side effects due to intracellular polymeric overloading, such ultrafine particles (sized around 0.1 μm) may be designed using polymers able to be degraded in vivo.

In certain aspects, a pharmaceutical composition comprising IL-15 may be administered to an individual in need thereof, in combination with one or more therapy provided herein, particularly one or more adenoviral vectors comprising nucleic acid sequences encoding one or more tumor antigens such as PSA, PSMA, MUC1, Brachyury, CEA, or a combination thereof.

Interleukin 15 (IL-15) is a cytokine with structural similarity to IL-2. Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells.

IL-15 can enhance the anti-tumor immunity of CD8+ T cells in pre-clinical models. A phase I clinical trial to evaluate the safety, dosing, and anti-tumor efficacy of IL-15 in patients with metastatic melanoma and renal cell carcinoma (kidney cancer) has begun to enroll patients at the National Institutes of Health.

IL-15 disclosed herein may also include mutants of IL-15 that are modified to maintain the function of its native form.

IL-15 is 14-15 kDa glycoprotein encoded by the 34 kb region 4q31 of chromosome 4, and by the central region of chromosome 8 in mice. The human IL-15 gene comprises nine exons (1-8 and 4A) and eight introns, four of which (exons 5 through 8) code for the mature protein. Two alternatively spliced transcript variants of this gene encoding the same protein have been reported. The originally identified isoform, with long signal peptide of 48 amino acids (IL-15 LSP) consisted of a 316 bp 5′-untranslated region (UTR), 486 bp coding sequence and the C-terminus 400 bp 3′-UTR region. The other isoform (IL-15 SSP) has a short signal peptide of 21 amino acids encoded by exons 4A and 5. Both isoforms shared 11 amino acids between signal sequences of the N-terminus. Although both isoforms produce the same mature protein, they differ in their cellular trafficking. IL-15 LSP isoform was identified in Golgi apparatus (GC), early endosomes and in the endoplasmic reticulum (ER). It exists in two forms, secreted and membrane-bound particularly on dendritic cells. On the other hand, IL-15 SSP isoform is not secreted and it appears to be restricted to the cytoplasm and nucleus where it plays an important role in the regulation of cell cycle.

It has been demonstrated that two isoforms of IL-15 mRNA are generated by alternatively splicing in mice. The isoform which had an alternative exon 5 containing another 3′ splicing site, exhibited a high translational efficiency, and the product lack hydrophobic domains in the signal sequence of the N-terminus. This suggests that the protein derived from this isoform is located intracellularly. The other isoform with normal exon 5, which is generated by integral splicing of the alternative exon 5, may be released extracellularly.

Although IL-15 mRNA can be found in many cells and tissues including mast cells, cancer cells or fibroblasts, this cytokine is produce as a mature protein mainly by dendritic cells, monocytes and macrophages. This discrepancy between the wide appearance of IL-15 mRNA and limited production of protein might be explained by the presence of the twelve in humans and five in mice upstream initiating codons, which can repress translation of IL-15 mRNA. Translational inactive mRNA is stored within the cell and can be induced upon specific signal. Expression of IL-15 can be stimulated by cytokine such as GM-CSF, double-strand mRNA, unmethylated CpG oligonucleotides, lipopolysaccharide (LPS) through Toll-like receptors (TLR), interferon gamma (IFN-γ) or after infection of monocytes herpes virus, Mycobacterium tuberculosis and Candida albicans.

### XI. Natural Killer (NK) Cells

In certain embodiments, native or engineered NK cells may be provided to be administered to a subject in need thereof, in combination with adenoviral vector-based compositions or immunotherapy as described herein.

The immune system is a tapestry of diverse families of immune cells each with its own distinct role in protecting from infections and diseases. Among these immune cells are the natural killer, or NK, cells as the body's first line of defense. NK cells have the innate ability to rapidly seek and destroy abnormal cells, such as cancer or virally-infected cells, without prior exposure or activation by other support molecules. In contrast to adaptive immune cells such as T cells, NK cells have been utilized as a cell-based “off-the-shelf” treatment in phase 1 clinical trials, and have demonstrated tumor killing abilities for cancer.

1. aNK Cells

In addition to native NK cells, there may be provided NK cells for administering to a patient that has do not express Killer Inhibitory Receptors (KIR), which diseased cells often exploit to evade the killing function of NK cells. This unique activated NK, or aNK, cell lack these inhibitory receptors while retaining the broad array of activating receptors which enable the selective targeting and killing of diseased cells. aNK cells also carry a larger pay load of granzyme and perforin containing granules, thereby enabling them to deliver a far greater payload of lethal enzymes to multiple targets.

2. taNK Cells

Chimeric antigen receptor (CAR) technology is among the most novel cancer therapy approaches currently in development. CARs are proteins that allow immune effector cells to target cancer cells displaying specific surface antigen (target-activated Natural Killer) is a platform in which aNK cells are engineered with one or more CARs to target proteins found on cancers and is then integrated with a wide spectrum of CARs. This strategy has multiple advantages over other CAR approaches using patient or donor sourced effector cells such as autologous T-cells, especially in terms of scalability, quality control and consistency.

Much of the cancer cell killing relies upon ADCC (antibody dependent cell-mediated cytotoxicity) whereupon effector immune cells attach to antibodies, which are in turn bound to the target cancer cell, thereby facilitating killing of the cancer by the effector cell. NK cells are the key effector cell in the body for ADCC and utilize a specialized receptor (CD16) to bind antibodies.

3. haNK Cells

Studies have shown that perhaps only 20% of the human population uniformly expresses the “high-affinity” variant of CD16 (haNK cells), which is strongly correlated with more favorable therapeutic outcomes compared to patients with the “low-affinity” CD16. Additionally, many cancer patients have severely weakened immune systems due to chemotherapy, the disease itself or other factors.

In certain aspects, NK cells are modified to express high-affinity CD16 (haNK cells). As such, haNK cells may potentiate the therapeutic efficacy of a broad spectrum of antibodies directed against cancer cells.

### XII. Combination Therapy

The compositions comprising an adenoviral vector-based vaccination comprising a nucleic acid sequence encoding tumor antigens such as PSA, PSMA, MUC1, Brachyury, CEA, or a combination thereof described throughout can be formulated into a pharmaceutical medicament and be used to treat a human or mammal in need thereof or diagnosed with a disease, e.g., cancer. These medicaments can be co-administered with one or more additional vaccines to a human or mammal, or together with one or more conventional cancer therapies or alternative cancer therapies, cytokines such as IL-15 or nucleic acid sequences encoding such cytokines, engineered natural killer cells, or immune pathway checkpoint modulators as described herein.

Conventional cancer therapies include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

In some embodiments, any vaccine described herein (e.g., Ad5 [E1−, E2b−]-HER3) can be combined with low dose chemotherapy or low dose radiation. For example, in some embodiment, any vaccine described herein (e.g., Ad5[E1−, E2b−]-HER3) can be combined with chemotherapy, such that the dose of chemotherapy administered is lower than the clinical standard of care. In some embodiments, the chemotherapy can be cyclophosphamide. The cyclophasmade can administered at a dose that is lower than the clinical standard of care dosing. For example, the chemotherapy can be administered at 50 mg twice a day (BID) on days 1-5 and 8-12 every 2 weeks for a total of 8 weeks. In some embodiments, any vaccine described herein (e.g., Ad5[E1−, E2b−]-HER3) can be combined with radiation, such that the dose of radiation administered is lower than the clinical standard of care. For example, in some embodiments, concurrent sterotactic body radiotherapy (SBRT) at 8 Gy can be given on day 8, 22, 36, 50 (every 2 weeks for 4 doses). Radiation can be administered to all feasible tumor sites using SBRT.

Radiation therapy that causes DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to, describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment described herein, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that treatment methods described herein may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, 7, 8, 9, 10, 11, 12, 13, or 14 days, or every 1, 2, 3, 4, 5, 6, 7, 8, 9, 10, 11, 12, 13, 14, 15, 16, 17, 18, 19, or 20 weeks, or every 1, 2, 3, 4, 5, 6, 7, 8, 9, 10, 11, 12, 13, 14, 15, 16, 18, 20, 22, 23, or 24 months. These treatments may be of varying dosages as well.

Alternative cancer therapies include any cancer therapy other than surgery, chemotherapy and radiation therapy, such as immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Gene therapy is the insertion of polynucleotides, including DNA or RNA, into a subject's cells and tissues to treat a disease. Antisense therapy is also a form of gene therapy. A therapeutic polynucleotide may be administered before, after, or at the same time of a first cancer therapy. Delivery of a vector encoding a variety of proteins is provided in some embodiments. For example, cellular expression of the exogenous tumor suppressor oncogenes would exert their function to inhibit excessive cellular proliferation, such as p53, p16 and C-CAM.

Additional agents to be used to improve the therapeutic efficacy of treatment include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with pharmaceutical compositions described herein to improve the anti-hyperproliferative efficacy of the treatments. Inhibitors of cell adhesion are contemplated to improve the efficacy of pharmaceutical compositions described herein. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with pharmaceutical compositions described herein to improve the treatment efficacy.

Hormonal therapy may also be used in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

A “Chemotherapeutic agent” or “chemotherapeutic compound” and their grammatical equivalents as used herein, can be a chemical compound useful in the treatment of cancer. The chemotherapeutic cancer agents that can be used in combination with the disclosed T cell include, but are not limited to, mitotic inhibitors (vinca alkaloids). These include vincristine, vinblastine, vindesine and Navelbine™ (vinorelbine, 5′-noranhydroblastine). In yet other embodiments, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide.

In certain aspects, methods or compositions described herein further encompass the use of other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein includes antibiotics. Examples include without limitation doxorubicin, bleomycin, dactinomycin, daunorubicin, mithramycin, mitomycin, mytomycin C, and daunomycin. There are numerous liposomal formulations commercially available for these compounds. In certain aspects, methods or compositions described herein further encompass the use of other chemotherapeutic cancer agents including without limitation anti-tumor antibodies, dacarbazine, azacytidine, amsacrine, melphalan, ifosfamide and mitoxantrone.

The disclosed adenovirus vaccine herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindcsine.

Additional formulations comprising population(s) of CAR T cells, T cell receptor engineered T cells, B cell receptor engineered cells, can be administered to a subject in conjunction, before, or after the administration of the pharmaceutical compositions described herein. A therapeutically-effective population of adoptively transferred cells can be administered to subjects when the methods described herein are practiced. In general, formulations are administered that comprise from about 1×10⁴ to about 1×10¹⁰ CAR T cells, T cell receptor engineered cells, or B cell receptor engineered cells. In some cases, the formulation comprises from about 1×10⁵ to about 1×10⁹ engineered cells, from about 5×10⁵ to about 5×10⁸ engineered cells, or from about 1×10⁶ to about 1×10⁷ engineered cells. However, the number of engineered cells administered to a subject will vary between wide limits, depending upon the location, source, identity, extent and severity of the cancer, the age and condition of the subject to be treated etc. A physician will ultimately determine appropriate dosages to be used.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

In some cases, for example, in the compositions, formulations and methods of treating cancer, the unit dosage of the composition or formulation administered can be 5, 10, 15, 20, 25, 30, 35, 40, 45, 50, 55, 60, 65, 70, 75, 80, 85, 90, 95, or 100 mg. In some cases, the total amount of the composition or formulation administered can be 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 40, 50, 60, 70, 80, 90, or 100 g.

### XIII. Immunological Fusion Partner Antigen Targets

The viral vectors or composition described herein may further comprise nucleic acid sequences that encode proteins, or an “immunological fusion partner,” that can increase the immunogenicity of the target antigen such as PSA and/or PSMA, or wherein the target antigen is any target antigen disclosed herein. In this regard, the protein produced following immunization with the viral vector containing such a protein may be a fusion protein comprising the target antigen of interest fused to a protein that increases the immunogenicity of the target antigen of interest. Furthermore, combination therapy with Ad5[E1−, E2b−] vectors encoding for PSA and/or PSMA and an immunological fusion partner can result in boosting the immune response, such that the combination of both therapeutic moieties acts to synergistically boost the immune response than either the Ad5[E1−, E2b−] vectors encoding for PSA and/or PSMA alone, or the immunological fusion partner alone. For example, combination therapy with Ad5[E1−, E2b−] vectors encoding for PSA and/or PSMA and an immunological fusion partner can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), antibody dependent cellular phagocytosis (ADCP) mechanisms, or any combination thereof. This synergistic boost can vastly improve survival outcomes after administration to a subject in need thereof. In certain embodiments, combination therapy with Ad5[E1−, E2b−] vectors encoding for PSA and/or PSMA and an immunological fusion partner can result in generating an immune response comprises an increase in target antigen-specific CTL activity of about 1.5 to 20, or more fold in a subject administered the adenovirus vectors as compared to a control. In another embodiment, generating an immune response comprises an increase in target-specific CTL activity of about 1.5 to 20, or more fold in a subject administered the Ad5[E1−, E2b−] vectors encoding for PSA and/or PSMA and an immunological fusion partner as compared to a control. In a further embodiment, generating an immune response that comprises an increase in target antigen-specific cell-mediated immunity activity as measured by ELISpot assays measuring cytokine secretion, such as interferon-gamma (IFN-γ), interleukin-2 (IL-2), tumor necrosis factor-alpha (TNF-α), or other cytokines, of about 1.5 to 20, or more fold as compared to a control. In a further embodiment, generating an immune response comprises an increase in target-specific antibody production of between 1.5 and 5 fold in a subject administered the Ad5[E1-, E2b−] vectors encoding for PSA and/or PSMA and an immunological fusion partner as described herein as compared to an appropriate control. In another embodiment, generating an immune response comprises an increase in target-specific antibody production of about 1.5 to 20, or more fold in a subject administered the adenovirus vector as compared to a control.

As an additional example, combination therapy with Ad5[E1−, E2b−] vectors encoding for target epitope antigens and an immunological fusion partner can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), antibody dependent cellular phagocytosis (ADCP) mechanisms, or any combination thereof. This synergistic boost can vastly improve survival outcomes after administration to a subject in need thereof. In certain embodiments, combination therapy with Ad5[E1−, E2b−] vectors encoding for target epitope antigens and an immunological fusion partner can result in generating an immune response comprises an increase in target antigen-specific CTL activity of about 1.5 to 20, or more fold in a subject administered the adenovirus vectors as compared to a control. In another embodiment, generating an immune response comprises an increase in target-specific CTL activity of about 1.5 to 20, or more fold in a subject administered the Ad5[E1−, E2b−] vectors encoding for target epitope antigens and an immunological fusion partner as compared to a control. In a further embodiment, generating an immune response that comprises an increase in target antigen-specific cell-mediated immunity activity as measured by ELISpot assays measuring cytokine secretion, such as interferon-gamma (IFN-γ), interleukin-2 (IL-2), tumor necrosis factor-alpha (TNF-α), or other cytokines, of about 1.5 to 20, or more fold as compared to a control. In a further embodiment, generating an immune response comprises an increase in target-specific antibody production of between 1.5 and 5 fold in a subject administered the adenovirus vectors as described herein as compared to an appropriate control. In another embodiment, generating an immune response comprises an increase in target-specific antibody production of about 1.5 to 20, or more fold in a subject administered the adenovirus vector as compared to a control.

In one embodiment, such an immunological fusion partner is derived from a Mycobacterium sp., such as a Mycobacterium tuberculosis-derived Ra12 fragment. The immunological fusion partner derived from Mycobacterium sp. can be any one of the sequences set forth in SEQ ID NO: 43-SEQ ID NO: 51. Ra12 compositions and methods for their use in enhancing the expression and/or immunogenicity of heterologous polynucleotide/polypeptide sequences are described in U.S. Pat. No. 7,009,042, which is herein incorporated by reference in its entirety. Briefly, Ra12 refers to a polynucleotide region that is a subsequence of a Mycobacterium tuberculosis MTB32A nucleic acid. MTB32A is a serine protease of 32 kDa encoded by a gene in virulent and avirulent strains of M. tuberculosis. The nucleotide sequence and amino acid sequence of MTB32A have been described (see, e.g., U.S. Pat. No. 7,009,042; Skeiky et al., Infection and Immun. 67:3998-4007 (1999), incorporated herein by reference in their entirety). C-terminal fragments of the MTB32A coding sequence can be expressed at high levels and remain as soluble polypeptides throughout the purification process. Moreover, Ra12 may enhance the immunogenicity of heterologous immunogenic polypeptides with which it is fused. A Ra12 fusion polypeptide can comprise a 14 kDa C-terminal fragment corresponding to amino acid residues 192 to 323 of MTB32A. Other Ra12 polynucleotides generally can comprise at least about 15, 30, 60, 100, 200, 300, or more nucleotides that encode a portion of a Ral2 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants can have at least about 70%, 80%, or 90% identity, or more, to a polynucleotide sequence that encodes a native Ra12 polypeptide or a portion thereof.

In certain aspects, an immunological fusion partner can be derived from protein D, a surface protein of the gram-negative bacterium Haemophilus influenzae B. The immunological fusion partner derived from protein D can be the sequence set forth in SEQ ID NO: 52. In some cases, a protein D derivative comprises approximately the first third of the protein (e.g., the first N-terminal 100-110 amino acids). A protein D derivative may be lipidated. Within certain embodiments, the first 109 residues of a Lipoprotein D fusion partner is included on the N-terminus to provide the polypeptide with additional exogenous T-cell epitopes, which may increase the expression level in E. coli and may function as an expression enhancer. The lipid tail may ensure optimal presentation of the antigen to antigen presenting cells. Other fusion partners can include the non-structural protein from influenza virus, NS1 (hemagglutinin). Typically, the N-terminal 81 amino acids are used, although different fragments that include T-helper epitopes may be used.

In certain aspects, the immunological fusion partner can be the protein known as LYTA, or a portion thereof (particularly a C-terminal portion). The immunological fusion partner derived from LYTA can the sequence set forth in SEQ ID NO: 53. LYTA is derived from Streptococcus pneumoniae, which synthesizes an N-acetyl-L-alanine amidase known as amidase LYTA (encoded by the LytA gene). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus can be employed. Within another embodiment, a repeat portion of LYTA may be incorporated into a fusion polypeptide. A repeat portion can, for example, be found in the C-terminal region starting at residue 178. One particular repeat portion incorporates residues 188-305.

In some embodiments, the target antigen is fused to an immunological fusion partner, also referred to herein as an “immunogenic component,” comprising a cytokine selected from the group of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The target antigen fusion can produce a protein with substantial identity to one or more of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The target antigen fusion can encode a nucleic acid encoding a protein with substantial identity to one or more of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the target antigen fusion further comprises one or more immunological fusion partner, also referred to herein as an “immunogenic components,” comprising a cytokine selected from the group of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The sequence of IFN-γ can be, but is not limited to, a sequence as set forth in SEQ ID NO: 54. The sequence of TNFα can be, but is not limited to, a sequence as set forth in SEQ ID NO: 55. The sequence of IL-2 can be, but is not limited to, a sequence as set forth in SEQ ID NO: 56. The sequence of IL-8 can be, but is not limited to, a sequence as set forth in SEQ ID NO: 57. The sequence of IL-12 can be, but is not limited to, a sequence as set forth in SEQ ID NO: 58. The sequence of IL-18 can be, but is not limited to, a sequence as set forth in SEQ ID NO: 59. The sequence of IL-7 can be, but is not limited to, a sequence as set forth in SEQ ID NO: 60. The sequence of IL-3 can be, but is not limited to, a sequence as set forth in SEQ ID NO: 61. The sequence of IL-4 can be, but is not limited to, a sequence as set forth in SEQ ID NO: 62. The sequence of IL-5 can be, but is not limited to, a sequence as set forth in SEQ ID NO: 63. The sequence of IL-6 can be, but is not limited to, a sequence as set forth in SEQ ID NO: 64. The sequence of IL-9 can be, but is not limited to, a sequence as set forth in SEQ ID NO: 65. The sequence of IL-10 can be, but is not limited to, a sequence as set forth in SEQ ID NO: 66. The sequence of IL-13 can be, but is not limited to, a sequence as set forth in SEQ ID NO: 67. The sequence of IL-15 can be, but is not limited to, a sequence as set forth in SEQ ID NO: 68. The sequence of IL-16 can be, but is not limited to, a sequence as set forth in SEQ ID NO: 95. The sequence of IL-17 can be, but is not limited to, a sequence as set forth in SEQ ID NO: 96. The sequence of IL-23 can be, but is not limited to, a sequence as set forth in SEQ ID NO: 97. The sequence of IL-32 can be, but is not limited to, a sequence as set forth in SEQ ID NO: 98.

In some embodiments, the target antigen is fused or linked to an immunological fusion partner, also referred to herein as an “immunogenic component,” comprising a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the target antigen is co-expressed in a cell with an immunological fusion partner, also referred to herein as an “immunogenic component,” comprising a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF.

In some embodiments, the target antigen is fused or linked to an immunological fusion partner, comprising CpG ODN (a non-limiting example sequence is shown in SEQ ID NO: 69), cholera toxin (a non-limiting example sequence is shown in SEQ ID NO: 70), a truncated A subunit coding region derived from a bacterial ADP-ribosylating exotoxin (a non-limiting example sequence is shown in (a non-limiting example sequence is shown in SEQ ID NO: 71), a truncated B subunit coding region derived from a bacterial ADP-ribosylating exotoxin (a non-limiting example sequence is shown in SEQ ID NO: 72), Hp91 (a non-limiting example sequence is shown in SEQ ID NO: 73), CCL20 (a non-limiting example sequence is shown in SEQ ID NO: 74), CCL3 (a non-limiting example sequence is shown in SEQ ID NO: 75), GM-CSF (a non-limiting example sequence is shown in SEQ ID NO: 76), G-CSF (a non-limiting example sequence is shown in SEQ ID NO: 77), LPS peptide mimic (non-limiting example sequences are shown in SEQ ID NO: 78-SEQ ID NO: 89), Shiga toxin (a non-limiting, example sequence is shown in SEQ ID NO: 90), diphtheria toxin (a non-limiting example sequence is shown in SEQ ID NO: 91), or CRM₁₉₇ (a non-limiting example sequence is shown in SEQ ID NO: 94).

In some embodiments, the target antigen is fused or linked to an immunological fusion partner, comprising an IL-15 superagonist. Interleukin 15 (IL-15) is a naturally occurring inflammatory cytokine secreted after viral infections. Secreted IL-15 can carry out its function by signaling via the its cognate receptor on effector immune cells, and thus, can lead to overall enhancement of effector immune cell activity.

Based on IL-15's broad ability to stimulate and maintain cellular immune responses, it is believed to be a promising immunotherapeutic drug that could potentially cure certain cancers. However, major limitations in clinical development of IL-15 can include low production yields in standard mammalian cell expression systems and short serum half-life. Moreover, the IL-15:IL-15Rα complex, comprising proteins co-expressed by the same cell, rather than the free IL-15 cytokine, can be responsible for stimulating immune effector cells bearing IL-15 βγc receptor.

To contend with these shortcomings, a novel IL-15 superagonist mutant (IL-15N72D) was identified that has increased ability to bind IL-15Rβγc and enhanced biological activity. Addition of either mouse or human IL-15Rα and Fc fusion protein (the Fc region of immunoglobulin) to equal molar concentrations of IL-15N72D can provide a further increase in IL-15 biologic activity, such that IL-15N72D:IL-15Rα/Fc super-agonist complex exhibits a median effective concentration (EC50) for supporting IL-15-dependent cell growth that was greater than 10-fold lower than that of free IL-15 cytokine.

In some embodiments, the IL-15 superagonist can be a novel IL-15 superagonist mutant (IL-15N72D). In certain embodiments, addition of either mouse or human IL-15Ra and Fc fusion protein (the Fc region of immunoglobulin) to equal molar concentrations of IL-15N72D can provide a further increase in IL-15 biologic activity, such that IL-15N72D:IL-15Rα/Fc super-agonist complex exhibits a median effective concentration (EC₅₀) for supporting IL-15-dependent cell growth that can be greater than 10-fold lower than that of free IL-15 cytokine.

Thus, in some embodiments, the present disclosure provides a IL-15N72D:IL-15Rα/Fc super-agonist complex with an EC50 for supporting IL-15-dependent cell growth that is greater than 2-fold lower, greater than 3-fold lower, greater than 4-fold lower, greater than 5-fold lower, greater than 6-fold lower, greater than 7-fold lower, greater than 8-fold lower, greater than 9-fold lower, greater than 10-fold lower, greater than 15-fold lower, greater than 20-fold lower, greater than 25-fold lower, greater than 30-fold lower, greater than 35-fold lower, greater than 40-fold lower, greater than 45-fold lower, greater than 50-fold lower, greater than 55-fold lower, greater than 60-fold lower, greater than 65-fold lower, greater than 70-fold lower, greater than 75-fold lower, greater than 80-fold lower, greater than 85-fold lower, greater than 90-fold lower, greater than 95-fold lower, or greater than 100-fold lower than that of free IL-15 cytokine.

In some embodiments, the IL-15 super agonist is a biologically active protein complex of two IL-15N72D molecules and a dimer of soluble IL-15Rα/Fc fusion protein, also known as ALT-803. The composition of ALT-803 and methods of producing and using ALT-803 are described in U.S. Patent Application Publication 2015/0374790, which is herein incorporated by reference. It is known that a soluble IL-15Rα fragment, containing the so-called “sushi” domain at the N terminus (Su), can bear most of the structural elements responsible for high affinity cytokine binding. A soluble fusion protein can be generated by linking the human IL-15RαSu domain (amino acids 1-65 of the mature human IL-15Rα protein) with the human IgG1 CH2-CH3 region containing the Fc domain (232 amino acids). This IL-15RαSu/IgG1 Fc fusion protein can have the advantages of dimer formation through disulfide bonding via IgG1 domains and ease of purification using standard Protein A affinity chromatography methods.

In some embodiments, ALT-803 can have a soluble complex consisting of 2 protein subunits of a human IL-15 variant associated with high affinity to a dimeric IL-15Rα sushi domain/human IgG1 Fc fusion protein. The IL-15 variant is a 114 amino acid polypeptide comprising the mature human IL-15 cytokine sequence with an Asn to Asp substitution at position 72 of helix C N72D). The human IL-15R sushi domain/human IgG1 Fc fusion protein comprises the sushi domain of the IL-15R subunit (amino acids 1-65 of the mature human IL-15Ra protein) linked with the human IgG1 CH2-CH3 region containing the Fc domain (232 amino acids). Aside from the N72D substitution, all of the protein sequences are human. Based on the amino acid sequence of the subunits, the calculated molecular weight of the complex comprising two IL-15N72D polypeptides (an example IL-15N72D sequence is shown in SEQ ID NO: 92) and a disulfide linked homodimeric IL-15RαSu/IgG1 Fc protein (an example IL-15RαSu/Fc domain is shown in SEQ ID NO: 93) is 92.4 kDa. In some embodiments, a recombinant vector encoding for a target antigen and for ALT-803 can have any sequence described herein to encode for the target antigen and can have SEQ ID NO: 92, SEQ ID NO: 92, SEQ ID NO: 93, and SEQ ID NO: 93 in any order, to encode for ALT-803.

Each IL-15N720 polypeptide has a calculated molecular weight of approximately 12.8 kDa and the IL-15RαSu/IgG 1 Fc fusion protein has a calculated molecular weight of approximately 33.4 kDa. Both the IL-15N72D and IL-15RαSu/IgG 1 Fc proteins can be glycosylated resulting in an apparent molecular weight of ALT-803 of approximately 114 kDa by size exclusion chromatography. The isoelectric point (pI) determined for ALT-803 can range from approximately 5.6 to 6.5. Thus, the fusion protein can be negatively charged at pH 7.

Combination therapy with Ad5[E1−, E2b−] vectors encoding for PSA and/or PSMA and ALT-803 can result in boosting the immune response, such that the combination of both therapeutic moieties acts to synergistically boost the immune response than either therapy alone. For example, combination therapy with Ad5[E1−, E2b−] vectors encoding for PSA and/or PSMA and ALT-803 can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), or antibody dependent cellular phagocytosis (ADCP) mechanisms. Combination therapy with Ad5[E1−, E2b−] vectors encoding for PSA and/or PSMA and ALT-803 can synergistically boost any one of the above responses, or a combination of the above responses, to vastly improve survival outcomes after administration to a subject in need thereof.

Any of the immunogenicity enhancing agents described herein can be fused or linked to a target antigen by expressing the immunogenicity enhancing agents and the target antigen in the same recombinant vector, using any recombinant vector described herein.

Nucleic acid sequences that encode for such immunogenicity enhancing agents can be any one of SEQ ID NO: 43-SEQ ID NO: 98 and are summarized in TABLE 1.

TABLE 1
Sequences of Immunogenicity Enhancing Agents

| SEQ ID NO | Sequence |
|---|---|
| SEQ ID NO: 43 | TAASDNFQLSQGGQGFAIPIGQAMAIAGQIRSG |
| | GGSPTVHIGPTAFLGLGVVDNNGNGARVQRVVG |
| | SAPAASLGISTGDVITAVDGAPINSATAMADAL |
| | NGHHPGDVISVTWQTKSGGTRTGNVTLAEGPPA |
| SEQ ID NO: 44 | MHHHHHHTAASDNFQLSQGGQGFAIPIGQAMAI |
| | AGQIRSGGGSPTVHIGPTAFLGLGVVDNNGNGA |
| | RVQRVVGSAPAASLGISTGDVITAVDGAPINSA |
| | TAMADALNGHHPGDVISVTWQTKSGGTRTGNVT |
| | LAEGPPAEFDDDDKDPPDPHQPDMTKGYCPGGR |
| | WGFGDLAVCDGEKYPDGSFWHQWMQTWFTGPQF |
| | YFDCVSGGEPLPGPPPPGGCGGAIPSEQPNAP |
| SEQ ID NO: 45 | MHHHHHHTAASDNFQLSQGGQGFAIPIGQAMAI |
| | AGQIRSGGGSPTVHIGPTAFLGLGVVDNNGNGA |
| | RVQRVVGSAPAASLGISTGDVITAVDGAPINSA |
| | TAMADALNGHHPGDVISVTWQTKSGGTRTGNVT |
| | LAEGPPAEFPLVPRGSPMGSDVRDLNALLPAVP |
| | SLGGGGGCALPVSGAAQWAPVLDFAPPGASAYG |
| | SLGGPAPPPAPPPPPPPPPHSFIKQEPSWGGAE |
| | PHEEQCLSAFTVHFSGQFTGTAGACRYGPFGPP |
| | PPSQASSGQARMFPNAPYLPSCLESQPAIRNQG |
| | YSTVTFDGTPSYGHTPSHHAAQFPNHSFKHEDP |
| | MGQQGSLGEQQYSVPPPVYGCHTPTDSCTGSQA |
| | LLLRTPYSSDNLYQMTSQLECMTWNQMNLGATL |
| | KGHSTGYESDNHTTPILCGAQYRIHTHGVFRGI |
| | QDVRRVPGVAPTLVRSASETSEKRPFMCAYSGC |
| | NKRYFKLSHLQMHSRKHTGEKPYQCDFKDCERR |
| | FFRSDQLKRHQRRHTGVKPFQCKTCQRKFSRSD |
| | HLKTHTRTHTGEKPFSCRWPSCQKKFARSDELV |
| | RHHNMHQRNMTKLQLAL |
| SEQ ID NO: 46 | MHHHHHHTAASDNFQLSQGGQGFAIPIGQAMAI |
| | AGQIRSGGGSPTVHIGPTAFLGLGVVDNNGNGA |
| | RVQRVVGSAPAASLGISTGDVITAVDGAPINSA |
| | TAMADALNGHHPGDVISVTWQTKSGGTRTGNVT |
| | LAEGPPAEFIEGRGSGCPLLENVISKTINPQVS |
| | KTEYKELLQEFIDDNATTNAIDELKECFLNQTD |
| | ETLSNVEVFMQLIYDSSLCDLF |
| SEQ ID NO: 47 | MHHHHHHTAASDNFQLSQGGQGFAIPIGQAMAI |
| | AGQIRSGGGSPTVHIGPTAFLGLGVVDNNGNGA |
| | RVQRVVGSAPAASLGISTGDVITAVDGAPINSA |
| | TAMADALNGHHPGDVISVTWQTKSGGTRTGNVT |
| | LAEGPPAEFMVDFGALPPEINSARMYAGPGSAS |
| | LVAAAQMWDSVASDLFSAASAFQSVVWGLTVGS |
| | WIGSSAGLMVAAASPYVAWMSVTAGQAELTAAQ |
| | VRVAAAAYETAYGLTVPPPVIAENRAELMILIA |
| | TNLLGQNTPAIAVNEAEYGEMWAQDAAAMFGYA |
| | AATATATATLLPFEEAPEMTSAGGLLEQAAAVE |
| | EASDTAAANQLMNNVPQALQQLAQPTQGTTPSS |
| | KLGGLWKTVSPHRSPISNMVSMANNHMSMTNSG |
| | VSMTNTLSSMLKGFAPAAAAQAVQTAAQNGVRA |
| | MSSLGSSLGSSGLGGGVAANLGRAASVGSLSVP |
| | QAWAAANQAVTPAARALPLTSLTSAAERGPGQM |
| | LGGLPVGQMGARAGGGLSGVLRVPPRPYVMPHS |
| | PAAGDIAPPALSQDRFADFPALPLDPSAMVAQV |
| | GPQVVNINTKLGYNNAVGAGTGIVIDPNGVVLT |
| | NNHVIAGATDINAFSVGSGQTYGVDVVGYDRTQ |
| | DVAVLQLRGAGGLPSAAIGGGVAVGEPVVAMGN |
| | SGGQGGTPRAVPGRVVALGQTVQASDSLTGAEE |
| | TLNGLIQFDAAIQPGDSGGPVVNGLGQVVGMNT |
| | AAS |
| SEQ ID NO: 48 | TAASDNFQLSQGGQGFAIPIGQAMAIAGQI |
| SEQ ID NO: 49 | TAASDNFQLSQGGQGFAIPIGQAMAIAGQIKLP |
| | TVHIGPTAFLGLGVVDNNGNGARVQRVVGSAPA |
| | ASLGISTGDVITAVDGAPINSATAMADALNGHH |
| | PGDVISVTWQTKSGGTRTGNVTLAEGPPA |
| SEQ ID NO: 50 | TAASDNFQLSQGGQGFAIPIGQAMAIAGQIRSG |
| | GGSPTVHIGPTAFLGLGVVDNNGNGARVQRVVG |
| | SAPAASLGISTGDVITAVDGAPINSATAMADAL |
| | NGHHPGDVISVTWQTKSGGTRTGNVTLAE |
| SEQ ID NO: 51 | MSNSRRRSLRWSWLLSVLAAVGLGLATAPAQAA |
| | PPALSQDRFADFPALPLDPSAMVAQVGPQVVNI |
| | NTKLGYNNAVGAGTGIVIDPNGVVLTNNHVIAG |
| | ATDINAFSVGSGQTYGVDVVGYDRTQDVAVLQL |
| | RGAGGLPSAAIGGGVAVGEPVVAMGNSGGQGGT |
| | PRAVPGRVVALGQTVQASDSLTGAEETLNGLIQ |
| | FDAAIQPGDSGGPVVNGLGQVVGMNTAASDNFQ |
| | LSQGGQGFAIPIGQAMAIAGQIRSGGGSPTVHI |
| | GPTAFLGLGVVDNNGNGARVQRVVGSAPAASLG |
| | ISTGDVITAVDGAPINSATAMADALNGHHPGDV |
| | ISVTWQTKSGGTRTGNVTLAEGPPA |
| SEQ ID NO: 52 | MKLKTLALSLLAAGVLAGCSSHSSNMANTQMKS |
| | DKIIIAHRGASGYLPEHTLESKALAFAQQADYL |
| | EQDLAMTKDGRLVVIHDHFLDGLTDVAKKFPHR |
| | HRKDGRYYVIDFTLKEIQSLEMTENFETKDGKQ |
| | AQVYPNRFPLWKSHFRIHTFEDEIEFIQGLEKS |
| | TGKKVGIYPEIKAPWFHHQNGKDIAAETLKVLK |
| | KYGYDKKTDMVYLQTFDFNELKRIKTELLPQMG |
| | MDLKLVQLIAYTDWKETQEKDPKGYWVNYNYDW |
| | MFICPGAMAEVVKYADGVGPGWYMLVNKEESKP |
| | DNIVYTPLVKELAQYNVEVHPYTVRKDALPAFF |
| | TDVNQMYDVLLNKSGATGVFTDFPDTGVEFLKG |
| | IK |
| SEQ ID NO: 53 | MEINVSKLRTDLPQVGVQPYRQVHAHSTGNPHS |
| | TVQNEADYHWRKDPELGFFSHIVGNGCIMQVGP |
| | VDNGAWDVGGGWNAETYAAVELIESHSTKEEFM |
| | TDYRLYIELLRNLADEAGLPKTLDTGSLAGIKT |
| | HEYCTNNQPNNHSDHVDPYPYLAKWGISREQFK |
| | HDIENGLTIETGWQKNDTGYWYVHSDGSYPKDK |
| | FEKINGTWYYFDSSGYMLADRWRKHTDGNWYWF |
| | DNSGEMATGWKKIADKWYYFNEEGAMKTGWVKY |
| | KDTWYYLDAKEGAMVSNAFIQSADGTGWYYLKP |
| | DGTLADRPEFRMSQMA |
| SEQ ID NO: 54 | MKYTSYILAFQLCIVLGSLGCYCQDPYVKEAEN |
| | LKKYFNAGHSDVADNGTLFLGILKNWKEESDRK |
| | IMQSQIVSFYFKLFKNFKDDQSIQKSVETIKED |
| | MNVKFFNSNKKKRDDFEKLTNYSVTDLNVQRKA |
| | IHELIQVMAELSPAAKTGKRKRSQMLFRGRRAS |
| | Q |
| SEQ ID NO: 55 | MSTESMIRDVELAEEALPKKTGGPQGSRRCLFL |
| | SLFSFLIVAGATTLFCLLHFGVIGPQREEFPRD |
| | LSLISPLAQAVRSSSRTPSDKPVAHVVANPQAE |
| | GQLQWLNRRANALLANGVELRDNQLVVPSEGLY |
| | LIYSQVLFKGQGCPSTHVLLTHTISRIAVSYQT |
| | KVNLLSAIKSPCQRETPEGAEAKPWYEPIYLGG |
| | VFQLEKGDRLSAEINRPDYLDFAESGQVYFGII |
| | AL |
| SEQ ID NO: 56 | MYRMQLLSCIALSLALVTNSAPTSSSTKKTQLQ |
| | LEHLLLDLQMILNGINNYKNPKLTRMLTFKFYM |
| | PKKATELKHLQCLEEELKPLEEVLNLAQSKNFH |
| | LRPRDLISNINVIVLELKGSETTFMCEYADETA |
| | TIVEFLNRWITFCQSIISTLT |
| SEQ ID NO: 57 | MTSKLAVALLAAFLISAALCEGAVLPRSAKELR |
| | CQCIKTYSKPFHPKFIKELRVIESGPHCANTEI |
| | IVKLSDGRELCLDPKENWVQRVVEKFLKRAENS |
| SEQ ID NO: 58 | MEPLVTWVVPLLFLFLLSRQGAACRTSECCFQD |
| | PPYPDADSGSASGPRDLRCYRISSDRYECSWQY |
| | EGPTAGVSHFLRCCLSSGRCCYFAAGSATRLQF |
| | SDQAGVSVLYTVTLWVESWARNQTEKSPEVTLQ |
| | LYNSVKYEPPLGDIKVSKLAGQLRMEWETPDNQ |
| | VGAEVQFRHRTPSSPWKLGDCGPQDDDTESCLC |
| | PLEMNVAQEFQLRRRQLGSQGSSWSKWSSPVCV |
| | PPENPPQPQVRFSVEQLGQDGRRRLTLKEQPTQ |
| | LELPEGCQGLAPGTEVTYRLQLHMLSCPCKAKA |
| | TRTLHLGKMPYLSGAAYNVAVISSNQFGPGLNQ |
| | TWHIPADTHTEPVALNISVGTNGTTMYWPARAQ |
| | SMTYCIEWQPVGQDGGLATCSLTAPQDPDPAGM |
| | ATYSWSRESGAMGQEKCYYITIFASAHPEKLTL |
| | WSTVLSTYHFGGNASAAGTPHHVSVKNHSLDSV |
| | SVDWAPSLLSTCPGVLKEYVVRCRDEDSKQVSE |
| | HPVQPTETQVTLSGLRAGVAYTVQVRADTAWLR |
| | GVWSQPQRFSIEVQVSDWLIFFASLGSFLSILL |
| | VGVLGYLGLNRAARHLCPPLPTPCASSAIEFPG |
| | GKETWQWINPVDFQEEASLQEALVVEMSWDKGE |
| | RTEPLEKTELPEGAPELALDTELSLEDGDRCKA |
| | KM |
| SEQ ID NO: 59 | MAAEPVEDNCINFVAMKFIDNTLYFIAEDDENL |
| | ESDYFGKLESKLSVIRNLNDQVLFIDQGNRPLF |
| | EDMTDSDCRDNAPRTIFIISMYKDSQPRGMAVT |
| | ISVKCEKISTLSCENKIISFKEMNPPDNIKDTK |
| | SDIIFFQRSVPGHDNKMQFESSSYEGYFLACEK |
| | ERDLFKLILKKEDELGDRSIMFTVQNED |
| SEQ ID NO: 60 | MFHVSFRYIFGLPPLILVLLPVASSDCDIEGKD |
| | GKQYESVLMVSIDQLLDSMKEIGSNCLNNEFNF |
| | FKRHKDANKEGMFLFRAARKLRQFLKMNSTGDF |
| | DLHLLKVSEGTTILLNCTGQVKGRKPAALGEAQ |
| | PTKSLEENKSLKEQKKLNDLCFLKRLLQEIKTC |
| | WNKILMGTKEH |
| SEQ ID NO: 61 | MSRLPVLLLLQLLVRPGLQAPMTQTTSLKTSWV |
| | NCSNMIDEIITHLKQPPLPLLDFNNLNGEDQDI |
| | LMENNLRRPNLEAFNRAVKSLQNASAIESILKN |
| | LLPCLPLATAAPTRHPIHIKDGDWNEFRRKLTF |
| | YLKTLENAQAQQTTLSLAIF |
| SEQ ID NO: 62 | MGLTSQLLPPLFFLLACAGNFVHGHKCDITLQE |
| | IIKTLNSLTEQKTLCTELTVTDIFAASKNTTEK |
| | ETFCRAATVLRQFYSHHEKDTRCLGATAQQFHR |
| | HKQURFLKRLDRNLWGLAGLNSCPVKEANQSTL |
| | ENFLERLKTIMREKYSKCSS |
| SEQ ID NO: 63 | MRMLLHLSLLALGAAYVYAIPTEIPTSALVKET |
| | LALLSTHRTLLIANETLRIPVPVHKNHQLCTEE |
| | IFQGIGTLESQTVQGGTVERLFKNLSLIKKYID |
| | GQKKKCGEERRRVNQFLDYLQEFLGVMNTEWII |
| | ES |
| SEQ ID NO: 64 | MNSFSTSAFGPVAFSLGLLLVLPAAFPAPVPPG |
| | EDSKDVAAPHRQPLTSSERIDKQIRYILDGISA |
| | LRKETCNKSNMCESSKEALAENNLNLPKMAEKD |
| | GCFQSGFNEETCLVKIITGLLEFEVYLEYLQNR |
| | FESSEEQARAVQMSTKVLIQFLQKKAKNLDAIT |
| | TPDPTTNASLLTKLQAQNQWLQDMTTHLILRSF |
| | KEFLQSSLRALRQM |
| SEQ ID NO: 65 | MVLTSALLLCSVAGQGCPTLAGILDINFLINKM |
| | QEDPASKCHCSANVTSCLCLGIPSDNCTRPCFS |
| | ERLSQMTNTTMQTRYPLIFSRVKKSVEVLKNNK |
| | CPYFSCEQPCNQTTAGNALTFLKSLLEIFQKEK |
| | MRGMRGKI |
| SEQ ID NO: 66 | MHSSALLCCLVLLTGVRASPGQGTQSENSCTHF |
| | PGNLPNMLRDLRDAFSRVKTFFQMKDQLDNLLL |
| | KESLLEDFKGYLGCQALSEMIQFYLEEVMPQAE |
| | NQDPDIKAHVNSLGENLKTLRLRLRRCHRFLPC |
| | ENKSKAVEQVKNAFNKLQEKGIYKAMSEFDIFI |
| | NYIEAYMTMKIRN |
| SEQ ID NO: 67 | MALLLTTVIALTCLGGFASPGPVPPSTALRELI |
| | EELVNITQNQKAPLCNGSMVWSINLTAGMYCAA |
| | LESLINVSGCSAIEKTQRMLSGFCPHKVSAGQF |
| | SSLHVRDTKIEVAQFVKDLLLHLKKLFREGQFN |
| | RNFESIIICRDRT |
| SEQ ID NO: 68 | MDFQVQIFSFLLISASVIMSRANWVNVISDLKK |
| | IEDLIQSMHIDATLYTESDVHPSCKVTAMKCFL |
| | LELQVISLESGDASIHDTVENLIILANNSLSSN |
| | GNVTESGCKECEELEEKNIKEFLQSFVHIVQMF |
| | INTS |
| SEQ ID NO: 69 | MEGDGSDPEPPDAGEDSKSENGENAPIYCKRKP |
| | DINCFMIGCDNCNEWFHGDCIRITEKMAKAIRE |
| | WYCRECREKDPKLEIRYRHKKSRERDGNERDSS |
| | EPRDEGGGRKRPVPDPNLQRRAGSGTGVGAMLA |
| | RGSASPHKSSPQPLVATPSQHHQQQQQQIKRSA |
| | RMCGECEACRRTEDCGHCDFCRDMKKFGGPNKI |
| | RQKCRLRQCQLRARESYKYFPSSLSPVTPSESL |
| | PRPRRPLPTQQQPQPSQKLGRIREDEGAVASST |
| | VKEPPEATATPEPLSDEDLPLDPDLYQDFCAGA |
| | FDDNGLPWMSDTEESPFLDPALRKRAVKVKHVK |
| | RREKKSEKKKEERYKRHRQKQKHKDKWKHPERA |
| | DAKDPASLPQCLGPGCVRPAQPSSKYCSDDCGM |
| | KLAANRIYEILPQRIQQWQQSPCIAEEHGKKLL |
| | ERIRREQQSARTRLQEMERRFHELEAIILRAKQ |
| | QAVREDEESNEGDSDDTDLQIFCVSCGHPINPR |
| | VALRHMERCYAKYESQTSFGSMYPTRIEGATRL |
| | FCDVYNPQSKTYCKRLQVLCPEHSRDPKVPADE |
| | VCGCPLVRDVFELTGDFCRLPKRQCNRHYCWEK |
| | LRRAEVDLERVRVWYKLDELFEQERNVRTAMTN |
| | RAGLLALMLHQTIQHDPLTTDLRSSADR |
| SEQ ID NO: 70 | MIKLKFGVFFTVLLSSAYAHGTPQNITDLCAEY |
| | HNTQIYTLNDKIFSYTESLAGKREMAIITFKNG |
| | AIFQVEVPGSQHIDSQKKAIERMKDTLRIAYLT |
| | EAKVEKLCVWNNKTPHAIAAISMAN |
| SEQ ID NO: 71 | MVKIIFVFFIFLSSFSYANDDKLYRADSRPPDE |
| | IKQSGGLMPRGQNEYFDRGTQMNINLYDHARGT |
| | QTGFVRHDDGYVSTSISLRSAHLVGQTILSGHS |
| | TYYIYVIATAPNMFNVNDVLGAYSPHPDEQEVS |
| | ALGGIPYSQIYGWYRVHFGVLDEQLHRNRGYRD |
| | RYYSNLDIAPAADGYGLAGFPPEHRAWREEPWI |
| | HHAPPGCGNAPRSSMSNTCDEKTQSLGVKFLDE |
| | YQSKVKRQIFSGYQSDIDTHNRIKDEL |
| SEQ ID NO: 72 | MIKLKFGVFFTVLLSSAYAHGTPQNITDLCAEY |
| | HNTQIHTLNDKILSYTESLAGNREMAIITFKNG |
| | ATFQVEVPGSQHIDSQKKAIERMKDTLRIAYLT |
| | EAKVEKLCVWNNKTPHAIAAISMAN |
| SEQ ID NO: 73 | DPNAPKRPPSAFFLFCSE |
| SEQ ID NO: 74 | MCCTKSLLLAALMSVLLLHLCGESEAASNFDCC |
| | LGYTDRILHPKFIVGFTRQLANEGCDINAIIFH |
| | TKKKLSVCANPKQTWVKYIVRLLSKKVKNM |
| SEQ ID NO: 75 | MQVSTAALAVLLCTMALCNQFSASLAADTPTAC |
| | CFSYTSRQIPQNFIADYFETSSQCSKPGVIFLT |
| | KRSRQVCADPSEEWVQKYVSDLELSA |
| SEQ ID NO: 76 | MWLQSLLLLGTVACSISAPARSPSPSTQPWEHV |
| | NAIQEARRLLNLSRDTAAEMNETVEVISEMFDL |
| | QEPTCLQTRLELYKQGLRGSLTKLKGPLTMMAS |
| | HYKQHCPPTPETSCATQIITFESFKENLKDFLL |
| | VIPFDCWEPVQE |
| SEQ ID NO: 77 | MAGPATQSPMKLMALQLLLWHSALWTVQEATPL |
| | GPASSLPQSFLLKCLEQVRKIQGDGAALQEKLC |
| | ATYKLCHPEELVLLGHSLGIPWAPLSSCPSQAL |
| | QLAGCLSQLHSGLFLYQGLLQALEGISPELGPT |
| | LDTLQLDVADFATTIWQQMEELGMAPALQPTQG |
| | AMPAFASAFQRRAGGVLVASHLQSFLEVSYRVL |
| | RHLAQP |
| SEQ ID NO: 78 | QEINSSY |
| SEQ ID NO: 79 | SHPRLSA |
| SEQ ID NO: 80 | SMPNPMV |
| SEQ ID NO: 81 | GLQQVLL |
| SEQ ID NO: 82 | HELSVLL |
| SEQ ID NO: 83 | YAPQRLP |
| SEQ ID NO: 84 | TPRTLPT |
| SEQ ID NO: 85 | APVHSSI |
| SEQ ID NO: 86 | APPHALS |
| SEQ ID NO: 87 | TFSNRFI |
| SEQ ID NO: 88 | VVPTPPY |
| SEQ ID NO: 89 | ELAPDSP |
| SEQ ID NO: 90 | TPDCVTGKVEYTKYNDDDTFTVKVGDKELFTNR |
| | WNLQSLLLSAQITGMTVTIKQNACHNGGGFSEV |
| | IFR |
| SEQ ID NO: 91 | MSRKLFASILIGALLGIGAPPSAHAGADDVVDS |
| | SKSFVMENFSSYHGTKPGYVDSIQKGIQKPKSG |
| | TQGNYDDDWKGFYSTDNKYDAAGYSVDNENPLS |
| | GKAGGVVKVTYPGLTKVLALKVDNAETIKKELG |
| | LSLTEPLMEQVGTEEFIKRFGDGASRVVLSLPF |
| | AEGSSSVEYINNWEQAKALSVELEINFETRGKR |
| | GQDAMYEYMAQACAGNRVRRSVGSSLSCINLDW |
| | DVIRDKTKTKIESLKEHGPIKNKMSESPNKTVS |
| | EEKAKQYLEEFHQTALEHPELSELKTVTGTNPV |
| | FAGANYAAWAVNVAQVIDSETADNLEKTTAALS |
| | ILPGIGSVMGIADGAVHHNTEEIVAQSIALSSL |
| | MVAQAIPLVGELVDIGFAAYNFVESIINLFQVV |
| | HNSYNRPAYSPGHKTQPFLHDGYAVSWNTVEDS |
| | IIRTGFQGESGHDIKITAENTPLPIAGVLLPTI |
| | PGKLDVNKSKTHISVNGRKIRMRCRAIDGDVTF |
| | CRPKSPVYVGNGVHANLHVAFHRSSSEKIHSNE |
| | ISSDSIGVLGYQKTVDHTKVNSKLSLFFEIKS |
| SEQ ID NO: 92 | NWVNVISDLKKIEDLIQSMHIDATLYTESDVHP |
| | SCKVTAMKCFLLELQVISLESGDASIHDTVENL |
| | IILANDSLSSNGNVTESGCKECEELEEKNIKEF |
| | LQSFVHIVQMFINTS |
| SEQ ID NO: 93 | ITCPPPMSVEHADIWVKSYSLYSRERYKNSGFK |
| | RKAGTSSLTECVLNKATNVAHWTTPSLKCIREP |
| | KSCDKTHTCPPCPAPELLGGPSVFLFPPKPKDT |
| | LMISRTPEVTCVVVDVSHEDPEVKFNWYVDGVE |
| | VHNAKTKPREEQYNSTYRVVSVLTVLHQDWLNG |
| | KEYKCKVSNKALPAPIEKTISKAKGQPREPQVY |
| | TLPPSRDELTKNQVSLTCLVKGFYPSDIAVEWE |
| | SNGQPENNYKTTPPVLDSDGSFFLYSKLTVDKS |
| | RWQQGNVFSCSVMHEALHNHYTQKSLSLSPGK |
| SEQ ID NO: 94 | GADDVVDSSKSFVMENFSSYHGTKPGYVDSIQK |
| | GIQKPKSGTQGNYDDDWKEFYSTDNKYDAAGYS |
| | VDNENPLSGKAGGVVKVTYPGLTKVLALKVDNA |
| | ETIKKELGLSLTEPLMEQVGTEEFIKRFGDGAS |
| | RVVLSLPFAEGSSSVEYINNWEQAKALSVELEI |
| | NFETRGKRGQDAMYEYMAQACAGNRVRRSVGSS |
| | LSCINLDWDVIRDKTKTKIESLKEHGPIKNKMS |
| | ESPNKTVSEEKAKQYLEEFHQTALEHPELSELK |
| | TVTGTNPVFAGANYAAWAVNVAQVIDSETADNL |
| | EKTTAALSILPGIGSVMGIADGAVHHNTEEIVA |
| | QSIALSSLMVAQAIPLVGELVDIGFAAYNFVES |
| | IINLFQVVHNSYNRPAYSPGHKTQPFLHDGYAV |
| | SWNTVEDSIIRTGFQGESGHDIKITAENTPLPI |
| | AGVLLPTIPGKLDVNKSKTHISVNGRKIRMRCR |
| | AIDGDVTFCRPKSPVYVGNGVHANLHVAFHRSS |
| | SEKIHSNEISSDSIGVLGYQKTVDHTKVNSKLS |
| | LFFEIKS |
| SEQ ID NO: 95 | MESHSRAGKSRKSAKFRSISRSLMLCNAKTSDD |
| | GSSPDEKYPDPFEISLAQGKEGIFHSSVQLADT |
| | SEAGPSSVPDLALASEAAQLQAAGNDRGKTCRR |
| | IFFMKESSTASSREKPGKLEAQSSNFLFPKACH |
| | QRARSNSTSVNPYCTREIDFPMTKKSAAPTDRQ |
| | PYSLCSNRKSLSQQLDCPAGKAAGTSRPTRSLS |
| | TAQLVQPSGGLQASVISNIVLMKGQAKGLGFSI |
| | VGGKDSIYGPIGIYVKTIFAGGAAAADGRLQEG |
| | DEILELNGESMAGLTHQDALQKFKQAKKGLLTL |
| | TVRTRLTAPPSLCSHLSPPLCRSLSSSTCITKD |
| | SSSFALESPSAPISTAKPNYRIMVEVSLQKEAG |
| | VGLGIGLCSVPYFQCISGIFVHTLSPGSVAHLD |
| | GRLRCGDEIVEISDSPVHCLTLNEVYTILSRCD |
| | PGPVPIIVSRHPDPQVSEQQLKEAVAQAVENTK |
| | FGKERHQWSLEGVKRLESSWHGRPTLEKEREKN |
| | SAPPHRRAQKVMIRSSSDSSYMSGSPGGSPGSG |
| | SAEKPSSDVDISTHSPSLPLAREPVVLSIASSR |
| | LPQESPPLPESRDSHPPLRLKKSFEILVRKPMS |
| | SKPKPPPRKYFKSDSDPQKSLEERENSSCSSGH |
| | TPPTCGQEARELLPLLLPQEDTAGRSPSASAGC |
| | PGPGIGPQTKSSTEGEPGWRRASPVTQTSPIKH |
| | PLLKRQARMDYSFDTTAEDPWVRISDCIKNLFS |
| | PIMSENHGHMPLQPNASLNEEEGTQGHPDGTPP |
| | KLDTANGTPKVYKSADSSTVKKGPPVAPKPAWF |
| | RQSLKGLRNRASDPRGLPDPALSTQPAPASREH |
| | LGSHIRASSSSSSIRQRISSFETFGSSQLPDKG |
| | AQRLSLQPSSGEAAKPLGKHEEGRFSGLLGRGA |
| | APTLVPQQPEQVLSSGSPAASEARDPGVSESPP |
| | PGRQPNQKTLPPGPDPLLRLLSTQAEESQGPVL |
| | KMPSQRARSFPLTRSQSCETKLLDEKTSKLYSI |
| | SSQVSSAVMKSLLCLPSSISCAQTPCIPKEGAS |
| | PTSSSNEDSAANGSAETSALDTGFSLNLSELRE |
| | YTEGLTEAKEDDDGDHSSLQSGQSVISLLSSEE |
| | LKKLIEEVKVLDEATLKQLDGIHVTILHKEEGA |
| | GLGFSLAGGADLENKVITVHRVFPNGLASQEGT |
| | IQKGNEVLSINGKSLKGTTHHDALAILRQAREP |
| | RQAVIVTRKLTPEAMPDLNSSTDSAASASAASD |
| | VSVESTEATVCTVTLEKMSAGLGFSLEGGKGSL |
| | HGDKPLTINRIFKGAASEQSETVQPGDEILQLG |
| | GTAMQGLTRFEAWNIIKALPDGPVTIVIRRKSL |
| | QSKETTAAGDS |
| SEQ ID NO: 96 | MTPGKTSLVSLLLLLSLEAIVKAGITIPRNPGC |
| | PNSEDKNFPRTVMVNLNIHNRNTNTNPKRSSDY |
| | YNRSTSPWNLHRNEDPERYPSVIWEAKCRHLGC |
| | INADGNVDYHMNSVPIQQEILVLRREPPHCPNS |
| | FRLEKILVSVGCTCVTPIVHHVA |
| SEQ ID NO: 97 | RAVPGGSSPAWTQCQQLSQKLCTLAWSAHPLVG |
| | HMDLREEGDEETTNDVPHIQCGDGCDPQGLRDN |
| | SQFCLQRIHQGLIFYEKLLGSDIFTGEPSLLPD |
| | SPVGQLHASLLGLSQLLQPEGHHWETQQIPSLS |
| | PSQPWQRLLLRFKILRSLQAFVAVAARVFAHGA |
| | ATLSPIWELKKDVYVVELDWYPDAPGEMVVLTC |
| | DTPEEDGITWTLDQSSEVLGSGKTLTIQVKEFG |
| | DAGQYTCHKGGEVLSHSLLLLHKKEDGIWSTDI |
| | LKDQKEPKNKTFLRCEAKNYSGRFTCWWLTTIS |
| | TDLTFSVKSSRGSSDPQGVTCGAATLSAERVRG |
| | DNKEYEYSVECQEDSACPAAEESLPIEVMVDAV |
| | HKLKYENYTSSFFIRDIIKPDPPKNLQLKPLKN |
| | SRQVEVSWEYPDTWSTPHSYFSLTFCVQVQGKS |
| | KREKKDRVFTDKTSATVKRKNASISVRAQDRYY |
| | SSSWSEWASVPCS |
| SEQ ID NO: 98 | MCFPKVLSDDMKKLKARMVMLLPTSAQGLGAWV |
| | SACDTEDTVGHLGPWRDKDPALWCQLCLSSQHQ |
| | AIERFYDKMQNAESGRGQVMSSLAELEDDFKEG |
| | YLETVAAYYEEQHPELTPLLEKERDGLRCRGNR |
| | SPVPDVEDPATEEPGESFCDKVMRWFQAMLQRL |
| | QTWWHGVLAWVKEKVVALVHAVQALWKQFQSFC |
| | CSLSELFMSSFQSYGAPRGDKEELTPQKCSEPQ |
| | SSK |

In some embodiments, the nucleic acid sequences for the target antigen and the immunological fusion partner are not separated by any nucleic acids. In other embodiments, a nucleic acid sequence that encodes for a linker can be inserted between the nucleic acid sequence encoding for any target antigen described herein and the nucleic acid sequence encoding for any immunological fusion partner described herein. Thus, in certain embodiments, the protein produced following immunization with the viral vector containing a target antigen, a linker, and an immunological fusion partner can be a fusion protein comprising the target antigen of interest followed by the linker and ending with the immunological fusion partner, thus linking the target antigen to an immunological fusion partner that increases the immunogenicity of the target antigen of interest via a linker. In some embodiments, the sequence of linker nucleic acids can be from about 1 to about 150 nucleic acids long, from about 5 to about 100 nucleic acids along, or from about 10 to about 50 nucleic acids in length. In some embodiments, the nucleic acid sequences may encode one or more amino acid residues. In some embodiments, the amino acid sequence of the linker can be from about 1 to about 50, or about 5 to about 25 amino acid residues in length. In some embodiments, the sequence of the linker comprises less than 10 amino acids. In some embodiments, the linker can be a polyalanine linker, a polyglycine linker, or a linker with both alanines and glycines.

Nucleic acid sequences that encode for such linkers can be any one of SEQ ID NO: 99-SEQ ID NO: 113 and are summarized in TABLE 2.

TABLE 2
Sequences of Linkers

| SEQ ID NO | Sequence |
|---|---|
| SEQ ID NO: 99 | MAVPMQLSCSR |
| SEQ ID NO: 100 | RSTG |
| SEQ ID NO: 101 | TR |
| SEQ ID NO: 102 | RSQ |
| SEQ ID NO: 103 | RSAGE |
| SEQ ID NO: 104 | RS |
| SEQ ID NO: 105 | GG |
| SEQ ID NO: 106 | GSGGSGGSG |
| SEQ ID NO: 107 | GGSGGSGGSGG |
| SEQ ID NO: 108 | GGSGGSGGSGGSGG |
| SEQ ID NO: 109 | GGSGGSGGSGGSGGSGG |
| SEQ ID NO: 110 | GGSGGSGGSGGSGGSGGSGG |
| SEQ ID NO: 111 | GGSGGSGGSGGSGGSGGSGGSGG |
| SEQ ID NO: 112 | GGSGGSGGSGGSGGSG |
| SEQ ID NO: 113 | GSGGSGGSGGSGGSGG |

### XIV. Costimulatory Molecules

In addition to the use of a recombinant adenovirus-based vector vaccine containing target antigens such as such as PSA, PSMA, MUC1, Brachyury, CEA, or a combination thereof, co-stimulatory molecules can be incorporated into said vaccine to increase immunogenicity. Initiation of an immune response requires at least two signals for the activation of naive T cells by APCs (Damle, et al. J Immunol 148:1985-92 (1992); Guinan, et al. Blood 84:3261-82 (1994); Hellstrom, et al. Cancer Chemother Pharmacol 38:S40-44 (1996); Hodge, et al. Cancer Res 39:5800-07 (1999)). An antigen specific first signal is delivered through the T cell receptor (TCR) via the peptide/major histocompatability complex (MHC) and causes the T cell to enter the cell cycle. A second, or costimulatory, signal may be delivered for cytokine production and proliferation.

At least three distinct molecules normally found on the surface of professional antigen presenting cells (APCs) have been reported as capable of providing the second signal critical for T cell activation: B7-1 (CD80), ICAM-1 (CD54), and LFA-3 (human CD58) (Damle, et al. J Immunol 148:1985-92 (1992); Guinan, et al. Blood 84: 3261-82 (1994); Wingren, et al. Crit Rev Immunol 15: 235-53 (1995); Parra, et al. Scand. J Immunol 38: 508-14 (1993); Hellstrom, et al. Ann NY Acad Sci 690: 225-30 (1993); Parra, et al. J Immunol 158: 637-42 (1997); Sperling, et al. J Immunol 157: 3909-17 (1996); Dubey, et al. J Immunol 155: 45-57 (1995); Cavallo, et al. Eur J Immunol 25: 1154-62 (1995)).

These costimulatory molecules have distinct T cell ligands. B7-1 interacts with the CD28 and CTLA-4 molecules, ICAM-1 interacts with the CD11a/CD18 (LFA-1132 integrin) complex, and LFA-3 interacts with the CD2 (LFA-2) molecules. Therefore, in a preferred embodiment, it would be desirable to have a recombinant adenovirus vector that contains B7-1, ICAM-1, and LFA-3, respectively, that, when combined with a recombinant adenovirus-based vector vaccine containing one or more nucleic acids encoding target antigens such as PSA, MUC1, Brachyury, CEA, or a combination thereof, will further increase/enhance anti-tumor immune responses directed to specific target antigens.

### XV. Immune Pathway Checkpoint Modulators

In certain embodiments, immune pathway checkpoint inhibitors, i.e., immune checkpoint inhibitors, are combined with compositions comprising adenoviral vectors disclosed herein. In certain embodiments, a patient received an immune pathway checkpoint inhibitor in conjunction with a vaccine or pharmaceutical compositions described herein. In further embodiments, compositions are administered with one or more immune pathway checkpoint modulators. A balance between activation and inhibitory signals regulates the interaction between T lymphocytes and disease cells, wherein T-cell responses are initiated through antigen recognition by the T-cell receptor (TCR). The inhibitory pathways and signals are referred to as immune pathway checkpoints. In normal circumstances, immune pathway checkpoints play a critical role in control and prevention of autoimmunity and also protect from tissue damage in response to pathogenic infection.

Certain embodiments provide combination immunotherapies comprising viral vector-based vaccines and compositions for modulating immune pathway checkpoint inhibitory pathways for the prevention and/or treatment of cancer and infectious diseases. In some embodiments, modulating is increasing expression or activity of a gene or protein. In some embodiments, modulating is decreasing expression or activity of a gene or protein. In some embodiments, modulating affects a family of genes or proteins.

In general, the immune inhibitory pathways are initiated by ligand-receptor interactions. It is now clear that in diseases, the disease can co-opt immune-checkpoint pathways as mechanism for inducing immune resistance in a subject.

The induction of immune resistance or immune inhibitory pathways in a subject by a given disease can be blocked by molecular compositions such as siRNAs, antisense, small molecules, mimic, a recombinant form of ligand, receptor or protein, or antibodies (which can be an Ig fusion protein) that are known to modulate one or more of the Immune Inhibitory Pathways. For example, preliminary clinical findings with blockers of immune-checkpoint proteins, such as Cytotoxic T-lymphocyte-associated antigen 4 (CTLA4) and programmed cell death protein 1 (PD1) have shown promise for enhancing anti-tumor immunity.

Because diseased cells can express multiple inhibitory ligands, and disease-infiltrating lymphocytes express multiple inhibitory receptors, dual or triple blockade of immune pathway checkpoints proteins may enhance anti-disease immunity. Combination immunotherapies as provide herein can comprise one or more compositions comprising an immune pathway checkpoint modulator that targets one or more of the following immune-checkpoint proteins: PD1, PDL1, PDL2, CD28, CD80, CD86, CTLA4, B7RP1, ICOS, B7RPI, B7-H3 (also known as CD276), B7-H4 (also known as B7-S1, B7x and VCTN1), BTLA (also known as CD272), HVEM, KIR, TCR, LAG3 (also known as CD223), CD137, CD137L, OX40, OX40L, CD27, CD70, CD40, CD40L, TIM3 (also known as HAVcr2), GALS, A2aR, and Adenosine.

In some embodiments, the molecular composition comprises a siRNAs. In some embodiments, the molecular composition comprises a small molecule. In some embodiments, the molecular composition comprises a recombinant form of a ligand. In some embodiments, the molecular composition comprises a recombinant form of a receptor. In some embodiments, the molecular composition comprises an antibody. In some embodiments, the combination therapy comprises more than one molecular composition and/or more than one type of molecular composition. As it will be appreciated by those in the art, future discovered proteins of the immune checkpoint inhibitory pathways are also envisioned to be encompassed by the present disclosure.

In some embodiments, combination immunotherapies comprise molecular compositions for the modulation of CTLA4. In some embodiments, combination immunotherapies comprise molecular compositions for the modulation of PD1. In some embodiments, combination immunotherapies comprise molecular compositions for the modulation of PDL1. In some embodiments, combination immunotherapies comprise molecular compositions for the modulation of LAG3. In some embodiments, combination immunotherapies comprise molecular compositions for the modulation of B7-H3. In some embodiments, combination immunotherapies comprise molecular compositions for the modulation of B7-H4. In some embodiments, combination immunotherapies comprise molecular compositions for the modulation of TIM3. In some embodiments, modulation is an increase or enhancement of expression. In other embodiments, modulation is the decrease of absence of expression.

Two non-limiting exemplary immune pathway checkpoint inhibitors include the cytotoxic T lymphocyte associated antigen-4 (CTLA-4) and the programmed cell death protein-1 (PD1). CTLA-4 can be expressed exclusively on T-cells where it regulates early stages of T-cell activation. CTLA-4 interacts with the co-stimulatory T-cell receptor CD28 which can result in signaling that inhibits T-cell activity. Once TCR antigen recognition occurs, CD28 signaling may enhances TCR signaling, in some cases leading to activated T-cells and CTLA-4 inhibits the signaling activity of CD28. The present disclosure provides immunotherapies as provided herein in combination with anti-CTLA-4 monoclonal antibody for the prevention and/or treatment of cancer and infectious diseases. The present disclosure provides vaccine or immunotherapies as provided herein in combination with CTLA-4 molecular compositions for the prevention and/or treatment of cancer and infectious diseases.

Programmed death cell protein ligand-1 (PDL1) is a member of the B7 family and is distributed in various tissues and cell types. PDL1 can interact with PD1 inhibiting T-cell activation and CTL mediated lysis. Significant expression of PDL1 has been demonstrated on various human tumors and PDL1 expression is one of the key mechanisms in which tumors evade host anti-tumor immune responses. Programmed death-ligand 1 (PDL1) and programmed cell death protein-1 (PD1) interact as immune pathway checkpoints. This interaction can be a major tolerance mechanism which results in the blunting of anti-tumor immune responses and subsequent tumor progression. PD1 is present on activated T cells and PDL1, the primary ligand of PD1, is often expressed on tumor cells and antigen-presenting cells (APC) as well as other cells, including B cells. Significant expression of PDL1 has been demonstrated on various human tumors including HPV-associated head and neck cancers. PDL1 interacts with PD1 on T cells inhibiting T cell activation and cytotoxic T lymphocyte (CTL) mediated lysis. The present disclosure provides immunotherapies as provided herein in combination with anti-PD1 or anti-PDL1 monoclonal antibody for the prevention and/or treatment of cancer and infectious diseases.

Certain embodiments may provide immunotherapies as provided herein in combination with PD1 or anti-PDL1 molecular compositions for the prevention and/or treatment of cancer and infectious diseases. Certain embodiments may provide immunotherapies as provided herein in combination with anti-CTLA-4 and anti-PD1 monoclonal antibodies for the prevention and/or treatment of cancer and infectious diseases. Certain embodiments may provide immunotherapies as provided herein in combination with anti-CTLA-4 and PDL1 monoclonal antibodies. Certain embodiments may provide vaccine or immunotherapies as provided herein in combination with anti-CTLA-4, anti-PD1, anti-PDL1 monoclonal antibodies, or a combination thereof, for the treatment of cancer and infectious diseases.

Immune pathway checkpoint molecules can be expressed by T cells. Immune pathway checkpoint molecules can effectively serve as “brakes” to down-modulate or inhibit an immune response. Immune pathway checkpoint molecules include, but are not limited to Programmed Death 1 (PD1 or PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as hepatitis A virus cellular receptor 2 (HAVCR2), GenBank accession number: JX049979.1), B and T lymphocyte associated (BTLA) (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1), SIGLECIO (GenBank accession number: AY358337.1), natural killer cell receptor 2B4 (also known as CD244, accession number: NM_001166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, ILIORA, IL10RB, HMOX2, IL6R, IL6ST, EIF2AK4, CSK, PAG1, SITZ, FOXP3, PRDM1, BATF, GUCY1A2, GUCY1A3, GUCY1B2, GUCY1B3 which directly inhibit immune cells. For example, PD1 can be combined with an adenoviral vector-based composition to treat a patient in need thereof.

Additional immune pathway checkpoints that can be targeted can be adenosine Ata receptor (ADORA), CD276, V-set domain containing T cell activation inhibitor 1 (VTCN1), indoleamine 2,3-dioxygenase 1 (IDO1), killer cell immunoglobulin-like receptor, three domains, long cytoplasmic tail, 1 (KIR3DL1), V-domain immunoglobulin suppressor of T-cell activation (VISTA), cytokine inducible SH2-containing protein (CISH), hypoxanthine phosphoribosyltransferase 1 (HPRT), adeno-associated virus integration site 1 (AAVS1), or chemokine (C—C motif) receptor 5 (gene/pseudogene) (CCR5), or any combination thereof.

TABLE 3, without being exhaustive, shows exemplary immune pathway checkpoint genes that can be inactivated to improve the efficiency of the adenoviral vector-based composition as described herein. Immune pathway checkpoints gene can be selected from such genes listed in TABLE 3 and others involved in co-inhibitory receptor function, cell death, cytokine signaling, arginine tryptophan starvation, TCR signaling, Induced T-reg repression, transcription factors controlling exhaustion or anergy, and hypoxia mediated tolerance.

TABLE 3
Exemplary immune pathway checkpoint genes

| Gene | NCBI # | | | Genome |
|---|---|---|---|---|
| Symbol | (GRCh38.p2) | Start | Stop | location |
| ADORA2A | 135 | 24423597 | 24442360 | 22q11.23 |
| CD276 | 80381 | 73684281 | 73714518 | 15q23-q24 |
| VTCN1 | 79679 | 117143587 | 117270368 | 1p13.1 |
| BTLA | 151888 | 112463966 | 112499702 | 3q13.2 |
| CTLA4 | 1493 | 203867788 | 203873960 | 2q33 |
| IDO1 | 3620 | 39913809 | 39928790 | 8p12-p11 |
| KIR3DL1 | 3811 | 54816438 | 54830778 | 19q13.4 |
| LAG3 | 3902 | 6772483 | 6778455 | 12p13.32 |
| PDCD1 | 5133 | 241849881 | 241858908 | 2q37.3 |
| HAVCR2 | 84868 | 157085832 | 157109237 | 5q33.3 |
| VISTA | 64115 | 71747556 | 71773580 | 10q22.1 |
| CD244 | 51744 | 160830158 | 160862902 | 1q23.3 |
| CISH | 1154 | 50606454 | 50611831 | 3p21.3 |

The combination of an adenoviral-based composition and an immune pathway checkpoint modulator may result in reduction in infection, progression, or symptoms of a disease in treated patients, as compared to either agent alone. In another embodiment, the combination of an adenoviral-based composition and an immune pathway checkpoint modulator may result in improved overall survival of treated patients, as compared to either agent alone. In some cases, the combination of an adenoviral-based composition and an immune pathway checkpoint modulator may increase the frequency or intensity of disease-specific T cell responses in treated patients as compared to either agent alone.

Certain embodiments may also provide the use of immune pathway checkpoint inhibition to improve performance of an adenoviral vector-based composition. Certain immune pathway checkpoint inhibitors may be administered at the time of an adenoviral vector-based composition. Certain immune pathway checkpoint inhibitors may also be administered after the administration of an adenoviral vector-based composition. Immune pathway checkpoint inhibition may occur simultaneously to an adenoviral vaccine administration. Immune pathway checkpoint inhibition may occur 1, 2, 3, 4, 5, 6, 7, 8, 9, 10, 15, 20, 30, 40, 50, or 60 minutes after vaccination. Immune pathway checkpoint inhibition may also occur 1, 2, 3, 4, 5, 6, 7, 8, 9, 10, 11, 12, 13, 14, 15, 16, 17, 18, 19, 20, 21, 22, 23, or 24 hours after the administration of an adenoviral vector-based composition. In some cases, immune inhibition may occur 1, 2, 3, 4, 5, 6, or 7 days after vaccination. Immune pathway checkpoint inhibition may occur at any time before or after the administration of an adenoviral vector-based composition.

In another aspect, there is provided methods involving a vaccine comprising one or more nucleic acids encoding an antigen and an immune pathway checkpoint modulator. For example, there is provided a method for treating a subject having a condition that would benefit from downregulation of an immune pathway checkpoint protein, PD1 or PDL1 for example, and its natural binding partner(s) on cells of the subject.

An immune pathway checkpoint modulator may be combined with an adenoviral vector-based composition comprising one or more nucleic acids encoding any antigen. For example, an antigen can be a tumor antigen, such as PSA, PSMA, MUC1, Brachyury, CEA, or a combination thereof, or any antigen described herein.

An immune pathway checkpoint modulator may produce a synergistic effect when combined with an adenoviral vector-based composition, such as a vaccine. An immune pathway checkpoint modulator may also produce a beneficial effect when combined with an adenoviral vector-based composition.

### XVI. Cancer

It is specifically contemplated that compositions comprising adenoviral vectors described herein can be used to evaluate or treat stages of disease, such as between hyperplasia, dysplasia, neoplasia, pre-cancer and cancer, or between a primary tumor and a metastasized tumor.

As used herein, the terms “neoplastic cells” and “neoplasia” may be used interchangeably and refer to cells which exhibit relatively autonomous growth, so that they exhibit an aberrant growth phenotype characterized by a significant loss of control of cell proliferation. Neoplastic cells can be malignant or benign. In particular aspects, a neoplasia includes both dysplasia and cancer. Neoplasms may be benign, pre-malignant (carcinoma in situ or dysplasia) or malignant (cancer). Neoplastic cells may form a lump (i.e., a tumor) or not.

The term “dysplasia” may be used when the cellular abnormality is restricted to the originating tissue, as in the case of an early, in-situ neoplasm. Dysplasia may be indicative of an early neoplastic process. The term “cancer” may refer to a malignant neoplasm, including a broad group of various diseases involving unregulated cell growth.

Metastasis, or metastatic disease, may refer to the spread of a cancer from one organ or part to another non-adjacent organ or part. The new occurrences of disease thus generated may be referred to as metastases.

Cancers that may be evaluated or treated by the disclosed methods and compositions include cancer cells particularly from the pancreas, including pancreatic ductal adenocarcinoma (PDAC), but may also include cells and cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcino ma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarco ma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### XVII. Methods of Treatment

The adenovirus vectors described herein can be used in a number of vaccine settings for generating an immune response against one or more target antigens as described herein. In some embodiments, there are provided methods of generating an immune response against any target antigen such as PSA, PSMA, MUC1, Brachyury, CEA, or a combination thereof.

The adenovirus vectors are of particular importance because of the unexpected finding that they can be used to generate immune responses in subjects who have preexisting immunity to Ad and can be used in vaccination regimens that include multiple rounds of immunization using the adenovirus vectors, regimens not possible using previous generation adenovirus vectors.

Generally, generating an immune response comprises an induction of a humoral response and/or a cell-mediated response. It may be desirable to increase an immune response against a target antigen of interest.

Generating an immune response may involve a decrease in the activity and/or number of certain cells of the immune system or a decrease in the level and/or activity of certain cytokines or other effector molecules. A variety of methods for detecting alterations in an immune response (e.g., cell numbers, cytokine expression, cell activity) are available and are useful in some aspects. Illustrative methods useful in this context include intracellular cytokine staining (ICS), ELISpot, proliferation assays, cytotoxic T-cell assays including chromium release or equivalent assays, and gene expression analysis using any number of polymerase chain reaction (PCR) or RT-PCR based assays.

Generating an immune response can comprise an increase in target antigen-specific CTL activity of from 1.5 to 5 fold in a subject administered the adenovirus vectors as described herein as compared to a control. In another embodiment, generating an immune response comprises an increase in target-specific CTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the adenovirus vectors as compared to a control.

Generating an immune response can comprise an increase in target antigen-specific HTL activity, such as proliferation of helper T-cells, of from 1.5 to 5 fold in a subject administered the adenovirus vectors as described herein that comprise nucleic acid encoding the target antigen as compared to an appropriate control. In another embodiment, generating an immune response comprises an increase in target-specific HTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold as compared to a control. In this context, HTL activity may comprise an increase as described above, or decrease, in production of a particular cytokine, such as interferon-γ (IFN-γ), interleukin-1 (IL-1), IL-2, IL-3, IL-6, IL-7, IL-12, IL-15, tumor necrosis factor-α (TNF-α), granulocyte macrophage colony-stimulating factor (GM-CSF), granulocyte-colony stimulating factor (G-CSF), or other cytokine. In this regard, generating an immune response may comprise a shift from a Th2 type response to a Th1 type response or in certain embodiments a shift from a Th1 type response to a Th2 type response. In other embodiments, generating an immune response may comprise the stimulation of a predominantly Th1 or a Th2 type response.

Generating an immune response can comprise an increase in target-specific antibody production of between 1.5 and 5 fold in a subject administered the adenovirus vectors as described herein as compared to an appropriate control. In another embodiment, generating an immune response comprises an increase in target-specific antibody production of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the adenovirus vector as compared to a control.

Thus, in certain embodiments, there are provided methods for generating an immune response against a target antigen of interest such as PSA, PSMA, MUC1, Brachyury, CEA, or a combination thereof comprising administering to the individual an adenovirus vector comprising: a) a replication defective adenovirus vector, wherein the adenovirus vector has a deletion in the E2b region, and b) a nucleic acid encoding the target antigen such as PSA, PSMA, MUC1, Brachyury, CEA, or a combination thereof; and readministering the adenovirus vector at least once to the individual; thereby generating an immune response against the target antigen. In certain embodiments, there are provided methods wherein the vector administered is not a gutted vector. In particular embodiments, the target antigen may be a wild-type protein, a fragment, a variant, or a variant fragment thereof. In some embodiments, the target antigen comprises a tumor antigen such as PSA, MUC1, Brachyury, CEA, or a combination thereof, a fragment, a variant, or a variant fragment thereof.

In a further embodiment, there are provided methods for generating an immune response against a target antigen in an individual, wherein the individual has preexisting immunity to Ad, by administering to the individual an adenovirus vector comprising: a) a replication defective adenovirus vector, wherein the adenovirus vector has a deletion in the E2b region, and b) a nucleic acid encoding the target antigen; and readministering the adenovirus vector at least once to the individual; thereby generating an immune response against the target antigen. In particular embodiments, the target antigen may be a wild-type protein, a fragment, a variant, or a variant fragment thereof. In some embodiments, the target antigen comprises such as PSA, PSMA, MUC1, Brachyury, CEA, or a combination thereof, a fragment, a variant, or a variant fragment thereof.

With regard to preexisting immunity to Ad, this can be determined using methods known in the art, such as antibody-based assays to test for the presence of Ad antibodies. Further, in certain embodiments, the methods as described herein include first determining that an individual has preexisting immunity to Ad then administering the E2b deleted adenovirus vectors as described herein.

One embodiment provides a method of generating an immune response against one or more target antigens in an individual comprising administering to the individual a first adenovirus vector comprising a replication defective adenovirus vector, wherein the adenovirus vector has a deletion in the E2b region, and a nucleic acid encoding at least one target antigen; administering to the individual a second adenovirus vector comprising a replication defective adenovirus vector, wherein the adenovirus vector has a deletion in the E2b region, and a nucleic acid encoding at least one target antigen, wherein the at least one target antigen of the second adenovirus vector is the same or different from the at least one target antigen of the first adenovirus vector. In particular embodiments, the target antigen may be a wild-type protein, a fragment, a variant, or a variant fragment thereof. In some embodiments, the target antigen comprises a tumor antigen such as PSA, PSMA, MUC1, Brachyury, CEA, or a combination thereof, a fragment, a variant, or a variant fragment thereof.

Thus, certain embodiments contemplate multiple immunizations with the same E2b deleted adenovirus vector or multiple immunizations with different E2b deleted adenovirus vectors. In each case, the adenovirus vectors may comprise nucleic acid sequences that encode one or more target antigens as described elsewhere herein. In certain embodiments, the methods comprise multiple immunizations with an E2b deleted adenovirus encoding one target antigen, and re-administration of the same adenovirus vector multiple times, thereby inducing an immune response against the target antigen. In some embodiments, the target antigen comprises a tumor antigen such as PSA, PSMA, MUC1, Brachyury, CEA, or a combination thereof, a fragment, a variant, or a variant fragment thereof.

In a further embodiment, the methods comprise immunization with a first adenovirus vector that encodes one or more target antigens, and then administration with a second adenovirus vector that encodes one or more target antigens that may be the same or different from those antigens encoded by the first adenovirus vector. In this regard, one of the encoded target antigens may be different or all of the encoded antigens may be different, or some may be the same and some may be different. Further, in certain embodiments, the methods include administering the first adenovirus vector multiple times and administering the second adenovirus multiple times. In this regard, the methods comprise administering the first adenovirus vector 1, 2, 3, 4, 5, 6, 7, 8, 9, 10, 11, 12, 13, 14, 15, or more times and administering the second adenovirus vector 1, 2, 3, 4, 5, 6, 7, 8, 9, 10, 11, 12, 13, 14, 15, or more times. The order of administration may comprise administering the first adenovirus one or multiple times in a row followed by administering the second adenovirus vector one or multiple times in a row. In certain embodiments, the methods include alternating administration of the first and the second adenovirus vectors as one administration each, two administrations each, three administrations each, and so on. In certain embodiments, the first and the second adenovirus vectors are administered simultaneously. In other embodiments, the first and the second adenovirus vectors are administered sequentially. In some embodiments, the target antigen comprises a tumor antigen such as PSA, PSMA, MUC1, Brachyury, CEA, or a combination thereof, a fragment, a variant, or a variant fragment thereof.

As would be readily understood by the skilled artisan, more than two adenovirus vectors may be used in the methods as described herein. Three, 4, 5, 6, 7, 8, 9, 10, or more different adenovirus vectors may be used in the methods as described herein. In certain embodiments, the methods comprise administering more than one E2b deleted adenovirus vector at a time. In this regard, immune responses against multiple target antigens of interest can be generated by administering multiple different adenovirus vectors simultaneously, each comprising nucleic acid sequences encoding one or more target antigens.

The adenovirus vectors can be used to generate an immune response against a cancer, such as carcinomas or sarcomas (e.g., solid tumors, lymphomas and leukemia). The adenovirus vectors can be used to generate an immune response against a cancer, such as neurologic cancers, melanoma, non-Hodgkin's lymphoma, Hodgkin's disease, leukemia, plasmocytomas, adenomas, gliomas, thymomas, breast cancer, prostate cancer, colorectal cancer, kidney cancer, renal cell carcinoma, uterine cancer, pancreatic cancer, esophageal cancer, lung cancer, ovarian cancer, cervical cancer, testicular cancer, gastric cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers.

Methods are also provided for treating or ameliorating the symptoms of any of the infectious diseases or cancers as described herein. The methods of treatment comprise administering the adenovirus vectors one or more times to individuals suffering from or at risk from suffering from an infectious disease or cancer as described herein. As such, certain embodiments provide methods for vaccinating against infectious diseases or cancers in individuals who are at risk of developing such a disease. Individuals at risk may be individuals who may be exposed to an infectious agent at some time or have been previously exposed but do not yet have symptoms of infection or individuals having a genetic predisposition to developing a cancer or being particularly susceptible to an infectious agent. Individuals suffering from an infectious disease or cancer described herein may be determined to express and/or present a target antigen, which may be use to guide the therapies herein. For example, an example can be found to express and/or present a target antigen and an adenovirus vector encoding the target antigen, a variant, a fragment or a variant fragment thereof may be administered subsequently.

Certain embodiments contemplate the use of adenovirus vectors for the in vivo delivery of nucleic acids encoding a target antigen, or a fragment, a variant, or a variant fragment thereof. Once injected into a subject, the nucleic acid sequence is expressed resulting in an immune response against the antigen encoded by the sequence. The adenovirus vector vaccine can be administered in an “effective amount,” that is, an amount of adenovirus vector that is effective in a selected route or routes of administration to elicit an immune response as described elsewhere herein. An effective amount can induce an immune response effective to facilitate protection or treatment of the host against the target infectious agent or cancer. The amount of vector in each vaccine dose is selected as an amount which induces an immune, immunoprotective or other immunotherapeutic response without significant adverse effects generally associated with typical vaccines. Once vaccinated, subjects may be monitored to determine the efficacy of the vaccine treatment. Monitoring the efficacy of vaccination may be performed by any method known to a person of ordinary skill in the art. In some embodiments, blood or fluid samples may be assayed to detect levels of antibodies. In other embodiments, ELISpot assays may be performed to detect a cell-mediated immune response from circulating blood cells or from lymphoid tissue cells.

In certain embodiments, from 1 to 10 doses may be administered over a 52 week period. In certain embodiments, 6 doses are administered, at intervals of 1, 2, 3, 4, 5, 6, 7, 8, 9, 10, 11, 12, 13, 14, 15, 16, 17, 18, 19, or 20 weeks, 1, 2, 3, 4, 5, 6, 7, 8, 9, 11, 12, 13; 14, 15, 16, 17, 18, 20, 22, 23, or 24 months or any range or value derivable therefrom, and further booster vaccinations may be given periodically thereafter, at intervals of 1, 2, 3, 4, 5, 6, 7, 8, 9, 10, 11, 12, 13, 14, 15, 16, 17, 18, 19, or 20 weeks, 1, 2, 3, 4, 5, 6, 7, 8, 9, 11, 12, 13, 14, 15, 16, 17, 18, 20, 22, 23, or 24 months or any range or value derivable therefrom. Alternate protocols may be appropriate for individual patients. As such, 1, 2, 3, 4, 5, 6, 7, 8, 9, 10, 11, 12, 13, 14, 15, 16, 17, 18, 19, 20, or more doses may be administered over a 1 year period or over shorter or longer periods, such as over 35, 40, 45, 50, 55, 60, 65, 70, 75, 80, 85, 90, 95, or 100 week periods. Doses may be administered at 1, 2, 3, 4, 5, or 6 week intervals or longer intervals.

A vaccine can be infused over a period of less than about 4 hours, and more preferably, over a period of less than about 3 hours. For example, the first 25-50 mg could be infused within 30 minutes, preferably even 15 min, and the remainder infused over the next 2-3 hrs. More generally, the dosage of an administered vaccine construct may be administered as one dosage every 2 or 3 weeks, repeated for a total of at least 3 dosages. Or, the construct may be administered twice per week for 4-6 weeks. The dosing schedule can optionally be repeated at other intervals and dosage may be given through various parenteral routes, with appropriate adjustment of the dose and schedule. Compositions as described herein can be administered to a patient in conjunction with (e.g., before, simultaneously, or following) any number of relevant treatment modalities.

A suitable dose is an amount of an adenovirus vector that, when administered as described above, is capable of promoting a target antigen immune response as described elsewhere herein. In certain embodiments, the immune response is at least 10-50% above the basal (i.e., untreated) level. In certain embodiments, the immune response is at least 2, 3, 4, 5, 6, 7, 8, 9, 10, 12, 15, 20, 25, 30, 35, 40, 45, 50, 55, 60, 65, 70, 75, 80, 85, 90, 100, 110, 125, 150, 200, 250, 300, 400, 500, or more over the basal level. Such response can be monitored by measuring the target antigen(s) antibodies in a patient or by vaccine-dependent generation of cytolytic effector cells capable of killing patient tumor or infected cells in vitro, or other methods known in the art for monitoring immune responses. Such vaccines should also be capable of causing an immune response that leads to an improved clinical outcome of the disease in question in vaccinated patients as compared to non-vaccinated patients. In some embodiments, the improved clinical outcome comprises treating disease, reducing the symptoms of a disease, changing the progression of a disease, or extending life.

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In some cases, the compositions provided herein are administered to a cell ex vivo. In some cases, the compositions provided herein are administered to an individual as a method of treating a disease or disorder. In some embodiments, the individual has a genetic disease. In some cases, the individual is at risk of having the disease, such as any of the diseases described herein. In some embodiments, the individual is at increased risk of having a disease or disorder caused by insufficient amount of a protein or insufficient activity of a protein. If an individual is “at an increased risk” of having a disease or disorder, the method involves preventative or prophylactic treatment. For example, an individual can be at an increased risk of having such a disease or disorder because of family history of the disease. Typically, individuals at an increased risk of having such a disease or disorder benefit from prophylactic treatment (e.g., by preventing or delaying the onset or progression of the disease or disorder).

In some cases, a subject does not have a disease. In some cases, the treatment as described herein is administered before onset of a disease. A subject may have undetected disease. A subject may have a low disease burden. A subject may also have a high disease burden. In certain cases, a subject may be administered a treatment as described herein according to a grading scale. A grading scale can be a Gleason classification. A Gleason classification reflects how different tumor tissue is from normal prostate tissue. It uses a scale from 1 to 5. A physician gives a cancer a number based on the patterns and growth of the cancer cells. The lower the number, the more normal the cancer cells look and the lower the grade. The higher the number, the less normal the cancer cells look and the higher the grade. In certain cases, a treatment may be administered to a patient with a low Gleason score. Preferably, a patient with a Gleason score of 3 or below may be administered a treatment as described herein.

Various embodiments relate to compositions and methods for raising an immune response against one or more particular target antigens such as PSA, PSMA, MUC1, Brachyury, CEA, or a combination thereof in selected patient populations. Accordingly, methods and compositions as described herein may target patients with a cancer including but not limited to prostate cancer, carcinomas or sarcomas such as neurologic cancers, melanoma, non-Hodgkin's lymphoma, Hodgkin's disease, leukemia, plasmocytomas, adenomas, gliomas, thymomas, breast cancer, colorectal cancer, kidney cancer, renal cell carcinoma, uterine cancer, pancreatic cancer, esophageal cancer, lung cancer, ovarian cancer, cervical cancer, testicular cancer, gastric cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers can be targeted for therapy.

In some cases, the targeted patient population may be limited to individuals having colorectal adenocarcinoma, metastatic colorectal cancer, advanced PSA, PSMA, MUC1, MUC1c, MUC1n, T, or CEA expressing cancer, prostate cancer, colorectal cancer, head and neck cancer, liver cancer, breast cancer, lung cancer, bladder cancer, or pancreas cancer. A histologically confirmed diagnosis of a selected cancer, for example colorectal adenocarcinoma, may be used. A particular disease stage or progression may be selected, for example, patients with one or more of a metastatic, recurrent, stage III, or stage IV cancer may be selected for therapy with the methods and compositions as described herein. In some embodiments, patients may be required to have received and, optionally, progressed through other therapies including but not limited to fluoropyrimidine, irinotecan, oxaliplatin, bevacizumab, cetuximab, or panitumumab containing therapies. In some cases, individual's refusal to accept such therapies may allow the patient to be included in a therapy eligible pool with methods and compositions as described herein. In some embodiments, individuals to receive therapy using the methods and compositions as described herein may be required to have an estimated life expectancy of at least, 1, 2, 3, 4, 5, 6, 7, 8, 9, 10, 11, 12, 14, 15, 18, 21, or 24 months. The patient pool to receive a therapy using the methods and compositions as described herein may be limited by age. For example, individuals who are older than 2, 3, 4, 5, 6, 7, 8, 9, 10, 11, 12, 13, 14, 15, 16, 17, 18, 19, 20, 21, 25, 30, 35, 40, 50, 60, or more years old can be eligible for therapy with methods and compositions as described herein. For another example, individuals who are younger than 75, 70, 65, 60, 55, 50, 40, 35, 30, 25, 20, or fewer years old can be eligible for therapy with methods and compositions as described herein.

In some embodiments, patients receiving therapy using the methods and compositions as described herein are limited to individuals with adequate hematologic function, for example with one or more of a white blood cell (WBC) count of at least 1000, 1500, 2000, 2500, 3000, 3500, 4000, 4500, 5000 or more per microliter, a hemoglobin level of at least 5, 6, 7, 8, 9, 10, 11, 12, 13, 14 or higher g/dL, a platelet count of at least 50,000; 60,000; 70,000; 75,000; 90,000; 100,000; 110,000; 120,000; 130,000; 140,000; 150,000 or more per microliter; with a PT-INR value of less than or equal to 0.8, 1.0, 1.2, 1.3, 1.4, 1.5, 1.6, 1.8, 2.0, 2.5, 3.0, or higher, a PTT value of less than or equal to 1.2, 1.4, 1.5, 1.6, 1.8, 2.0×ULN or more. In various embodiments, hematologic function indicator limits are chosen differently for individuals in different gender and age groups, for example 0-5, 5-10, 10-15, 15-18, 18-21, 21-30, 30-40, 40-50, 50-60, 60-70, 70-80, or older than 80.

In some embodiments, patients receiving therapy using the methods and compositions as described herein are limited to individuals with adequate renal and/or hepatic function, for example with one or more of a serum creatinine level of less than or equal to 0.8, 0.9, 1.0, 1.1, 1.2, 1.3, 1.4, 1.5, 1.6, 1.7, 1.8, 1.9, 2.0, 2.1, 2.2 mg/dL, or more, a bilirubin level of 0.8, 0.9, 1.0, 1.1, 1.2, 1.3, 1.4, 1.5, 1.6, 1.7, 1.8, 1.9, 2.0, 2.1, 2.2 mg/dL, or more, while allowing a higher limit for Gilbert's syndrome, for example, less than or equal to 1.5, 1.6, 1.8, 1.9, 2.0, 2.1, 2.2, 2.3, or 2.4 mg/dL, an ALT and AST value of less than or equal to less than or equal to 1.5, 2.0, 2.5, 3.0× upper limit of normal (ULN) or more. In various embodiments, renal or hepatic function indicator limits are chosen differently for individuals in different gender and age groups, for example 0-5, 5-10, 10-15, 15-18, 18-21, 21-30, 30-40, 40-50, 50-60, 60-70, 70-80, or older than 80.

In some embodiments, the K-ras mutation status of individuals who are candidates for a therapy using the methods and compositions as described herein can be determined. Individuals with a preselected K-ras mutational status can be included in an eligible patient pool for therapies using the methods and compositions as described herein.

In various embodiments, patients receiving therapy using the methods and compositions as described herein are limited to individuals without concurrent cytotoxic chemotherapy or radiation therapy, a history of, or current, brain metastases, a history of autoimmune disease, such as but not restricted to, inflammatory bowel disease, systemic lupus erythematosus, ankylosing spondylitis, scleroderma, multiple sclerosis, thyroid disease and vitiligo, serious intercurrent chronic or acute illness, such as cardiac disease (NYHA class III or IV), or hepatic disease, a medical or psychological impediment to probable compliance with the protocol, concurrent (or within the last 5 years) second malignancy other than non-melanoma skin cancer, cervical carcinoma in situ, controlled superficial bladder cancer, or other carcinoma in situ that has been treated, an active acute or chronic infection including: a urinary tract infection, HIV (e.g., as determined by ELISA and confirmed by Western Blot), and chronic hepatitis, or concurrent steroid therapy (or other immuno-suppressives, such as azathioprine or cyclosporin A). In some cases, patients with at least 3, 4, 5, 6, 7, 8, 9, or 10 weeks of discontinuation of any steroid therapy (except that used as pre-medication for chemotherapy or contrast-enhanced studies) may be included in a pool of eligible individuals for therapy using the methods and compositions as described herein. In some embodiments, patients receiving therapy using the methods and compositions of as described herein include individuals with thyroid disease and vitiligo.

In various embodiments, samples, for example serum or urine samples, from the individuals or candidate individuals for a therapy using the methods and compositions as described herein may be collected. Samples may be collected before, during, and/or after the therapy for example, within 2, 4, 6, 8, 10 weeks prior to the start of the therapy, within 1 week, 10 day, 2 weeks, 3 weeks, 4 weeks, 6 weeks, 8 weeks, or 12 weeks from the start of the therapy, within 2, 4, 6, 8, 10 weeks prior to the start of the therapy, within 1 week, 10 day, 2 weeks, 3 weeks, 4 weeks, 6 weeks, 8 weeks, 9 weeks, or 12 weeks from the start of the therapy, in 1 week, 10 day, 2 weeks, 3 weeks, 4 weeks, 6 weeks, 8 weeks, 9 weeks, or 12 weeks intervals during the therapy, in 1 month, 3 month, 6 month, 1 year, 2 year intervals after the therapy, within 1 month, 3 months, 6 months, 1 year, 2 years, or longer after the therapy, for a duration of 6 months, 1, 2, 3, 4, 5, 6, 7, 8, 9, 10 years, or longer. The samples may be tested for any of the hematologic, renal, or hepatic function indicators described herein as well as suitable others known in the art, for example a β-HCG for women with childbearing potential. In that regard, hematologic and biochemical tests, including cell blood counts with differential, PT, INR and PTT, tests measuring Na, K, Cl, CO₂, BUN, creatinine, Ca, total protein, albumin, total bilirubin, alkaline phosphatase, AST, ALT and glucose are contemplated in certain aspects. In some embodiments, the presence or the amount of HIV antibody, Hepatitis BsAg, or Hepatitis C antibody are determined in a sample from individuals or candidate individuals for a therapy using the methods and compositions described herein.

Biological markers, such as antibodies to target antigens or the neutralizing antibodies to Ad5 vector can be tested in a sample, such as serum, from individuals or candidate individuals for a therapy using the methods and compositions described herein. In some cases, one or more samples, such as a blood sample can be collected and archived from an individuals or candidate individuals for a therapy using the methods and compositions described herein. Collected samples can be assayed for immunologic evaluation. Individuals or candidate individuals for a therapy using the methods and compositions described herein can be evaluated in imaging studies, for example using CT scans or MRI of the chest, abdomen, or pelvis. Imaging studies can be performed before, during, or after therapy using the methods and compositions described herein, during, and/or after the therapy, for example, within 2, 4, 6, 8, 10 weeks prior to the start of the therapy, within 1 week, 10 day, 2 weeks, 3 weeks, 4 weeks, 6 weeks, 8 weeks, or 12 weeks from the start of the therapy, within 2, 4, 6, 8, 10 weeks prior to the start of the therapy, within 1 week, 10 day, 2 weeks, 3 weeks, 4 weeks, 6 weeks, 8 weeks, 9 weeks, or 12 weeks from the start of the therapy, in 1 week, 10 day, 2 week, 3 week, 4 week, 6 week, 8 week, 9 week, or 12 week intervals during the therapy, in 1 month, 3 month, 6 month, 1 year, 2 year intervals after the therapy, within 1 month, 3 months, 6 months, 1 year, 2 years, or longer after the therapy, for a duration of 6 months, 1, 2, 3, 4, 5, 6, 7, 8, 9, 10 years, or longer.

Compositions and methods described herein contemplate various dosage and administration regimens during therapy. Patients may receive one or more replication defective adenovirus or adenovirus vector, for example, Ad5 [E1−, E2B−]-vectors comprising a target antigen that is capable of raising an immune response in an individual against a target antigen described herein.

In various embodiments, the replication defective adenovirus is administered at a dose that suitable for effecting such immune response. In some embodiments, the replication defective adenovirus is administered at a dose from about 1×10⁸ virus particles to about 5×10¹³ virus particles per immunization. In some cases, the replication defective adenovirus is administered at a dose that is from about 1×10⁹ to about 5×10¹² virus particles per immunization. In some embodiments, the replication defective adenovirus is administered at a dose from about 1×10⁸ virus particles to about 5×10⁸ virus particles per immunization. In some embodiments, the replication defective adenovirus is administered at a dose from about 5×10⁸ virus particles to about 1×10⁹ virus particles per immunization. In some embodiments, the replication defective adenovirus is administered at a dose from about 1×10⁹ virus particles to about 5×10⁹ virus particles per immunization. In some embodiments, the replication defective adenovirus is administered at a dose from about 5×10⁹ virus particles to about 1×10¹⁰ virus particles per immunization. In some embodiments, the replication defective adenovirus is administered at a dose from about 1×10¹⁰ virus particles to about 5×10¹⁰ virus particles per immunization. In some embodiments, the replication defective adenovirus is administered at a dose from about 5×10¹⁰ virus particles to about 1×10¹¹ virus particles per immunization. In some embodiments, the replication defective adenovirus is administered at a dose from about 1×10¹¹ virus particles to about 5×10¹¹ virus particles per immunization. In some embodiments, the replication defective adenovirus is administered at a dose from about 5×10¹¹ virus particles to about 1×10¹² virus particles per immunization. In some embodiments, the replication defective adenovirus is administered at a dose from about 1×10¹² virus particles to about 5×10¹² virus particles per immunization. In some embodiments, the replication defective adenovirus is administered at a dose from about 5×10¹² virus particles to about 1×10¹³ virus particles per immunization. In some embodiments, the replication defective adenovirus is administered at a dose from about 1×10¹³ virus particles to about 5×10¹³ virus particles per immunization. In some embodiments, the replication defective adenovirus is administered at a dose from about 1×10⁸ virus particles to about 5×10¹⁰ virus particles per immunization. In some embodiments, the replication defective adenovirus is administered at a dose from about 1×10¹⁰ virus particles to about 5×10¹² virus particles per immunization. In some embodiments, the replication defective adenovirus is administered at a dose from about 1×10¹¹ virus particles to about 5×10¹³ virus particles per immunization. In some embodiments, the replication defective adenovirus is administered at a dose from about 1×10⁸ virus particles to about 1×10¹⁰ virus particles per immunization. In some embodiments, the replication defective adenovirus is administered at a dose from about 1×10¹⁰ virus particles to about 1×10¹² virus particles per immunization. In some embodiments, the replication defective adenovirus is administered at a dose from about 1×10¹¹ virus particles to about 5×10¹³ virus particles per immunization. In some cases, the replication defective adenovirus is administered at a dose that is greater than or equal to 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 3×10¹², or more virus particles (VP) per immunization. In some cases, the replication defective adenovirus is administered at a dose that is less than or equal to 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 3×10¹², or more virus particles per immunization. In various embodiments, a desired dose described herein is administered in a suitable volume of formulation buffer, for example a volume of about 0.1-10 mL, 0.2-8 mL, 0.3-7 mL, 0.4-6 mL, 0.5-5 mL, 0.6-4 mL, 0.7-3 mL, 0.8-2 mL, 0.9-1.5 mL, 0.95-1.2 mL, or 1.0-1.1 mL. Those of skill in the art appreciate that the volume may fall within any range bounded by any of these values (e.g., about 0.5 mL to about 1.1 mL). Administration of virus particles can be through a variety of suitable paths for delivery, for example it can be by injection (e.g., intracutaneously, intramuscularly, intravenously or subcutaneously), intranasally (e.g., by aspiration), in pill form (e.g., swallowing, suppository for vaginal or rectal delivery. In some embodiments, a subcutaneous delivery may be preferred and can offer greater access to dendritic cells.

Administration of virus particles to an individual may be repeated. Repeated deliveries of virus particles may follow a schedule or alternatively, may be performed on an as needed basis. For example, an individual's immunity against a target antigen, for example, a tumor antigen such as PSA, PSMA, MUC1, Brachyury, CEA, or a combination thereof, a fragment, a variant, or a variant fragment thereof, may be tested and replenished as necessary with additional deliveries. In some embodiments, schedules for delivery include administrations of virus particles at regular intervals. Joint delivery regimens may be designed comprising one or more of a period with a schedule and/or a period of need based administration assessed prior to administration. For example, a therapy regimen may include an administration, such as subcutaneous administration once every three weeks then another immunotherapy treatment every three months until removed from therapy for any reason including death. Another example regimen comprises three administrations every three weeks then another set of three immunotherapy treatments every three months.

Another example regimen comprises a first period with a first number of administrations at a first frequency, a second period with a second number of administrations at a second frequency, a third period with a third number of administrations at a third frequency, etc., and optionally one or more periods with undetermined number of administrations on an as needed basis. The number of administrations in each period can be independently selected and can for example be 1, 2, 3, 4, 5, 6, 7, 8, 9, 10, 11, 12, 13, 14, 15, 16, 17, 18, 19, 20, or more. The frequency of the administration in each period can also be independently selected, can for example be about every day, every other day, every third day, twice a week, once a week, once every other week, every three weeks, every month, every six weeks, every other month, every third month, every fourth month, every fifth month, every sixth month, once a year etc. The therapy can take a total period of up to 1, 2, 3, 4, 5, 6, 7, 8, 9, 10, 11, 12, 13, 14, 15, 16, 17, 18, 19, 20, 21, 22, 23, 24, 30, 36 months, or more.

The scheduled interval between immunizations may be modified so that the interval between immunizations is revised by up to a fifth, a fourth, a third, or half of the interval. For example, for a 3-week interval schedule, an immunization may be repeated from 20 to 28 days (3 weeks−1 day to 3 weeks+7 days). For the first 3 immunizations, if the second and/or third immunization is delayed, the subsequent immunizations may be shifted allowing a minimum amount of buffer between immunizations. For example, for a three-week interval schedule, if an immunization is delayed, the subsequent immunization may be scheduled to occur no earlier than 17, 18, 19, or 20 days after the previous immunization.

Compositions described herein can be provided in various states, for example, at room temperature, on ice, or frozen. Compositions may be provided in a container of a suitable size, for example a vial of 2 mL vial. In one embodiment, a 2 ml vial with 1.0 mL of extractable vaccine contains 5×10¹¹ total virus particles/mL. Storage conditions including temperature and humidity may vary. For example, compositions for use in therapy may be stored at room temperature, 4° C., −20° C., or lower.

In various embodiments, general evaluations are performed on the individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

General evaluations may include one or more of medical history, ECOG Performance Score, Karnofsky performance status, and complete physical examination with weight by the attending physician. Any other treatments, medications, biologics, or blood products that the patient is receiving or has received since the last visit may be recorded. Patients may be followed at the clinic for a suitable period, for example approximately 30 minutes, following receipt of vaccine to monitor for any adverse reactions.

In certain embodiments, local and systemic reactogenicity after each dose of vaccine may be assessed daily for a selected time, for example for 3 days (on the day of immunization and 2 days thereafter). Diary cards may be used to report symptoms and a ruler may be used to measure local reactogenicity. Immunization injection sites may be assessed. CT scans or MRI of the chest, abdomen, and pelvis may be performed.

In various embodiments, hematological and biochemical evaluations are performed on the individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization. Hematological and biochemical evaluations may include one or more of blood test for chemistry and hematology, CBC with differential, Na, K, Cl, CO₂, BUN, creatinine, Ca, total protein, albumin, total bilirubin, alkaline phosphatase, AST, ALT, glucose, and ANA.

In various embodiments, biological markers are evaluated on individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

Biological marker evaluations may include one or more of measuring antibodies to target antigens or viral vectors described herein, from a serum sample of adequate volume, for example about 5 ml Biomarkers may be reviewed if determined and available.

In various embodiments, an immunological assessment is performed on individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

Peripheral blood, for example about 90 mL may be drawn prior to each immunization and at a time after at least some of the immunizations, to determine whether there is an effect on the immune response at specific time points during the study and/or after a specific number of immunizations. Immunological assessment may include one or more of assaying peripheral blood mononuclear cells (PBMC) for T-cell responses to target antigens using ELISpot, proliferation assays, multi-parameter flow cytometric analysis, and cytoxicity assays. Serum from each blood draw may be archived and sent and determined.

In various embodiments, a tumor assessment is performed on individuals receiving treatment according to the methods and compositions as described herein. One or more of any tests may be performed as needed or in a scheduled basis, such as prior to treatment, on weeks 0, 3, 6, etc. A different set of tests may be performed concurrent with immunization vs. at time points without immunization. Tumor assessment may include one or more of CT or MRI scans of chest, abdomen, or pelvis performed prior to treatment, at a time after at least some of the immunizations and at approximately every three months following the completion of a selected number, for example 2, 3, or 4, of first treatments and for example until removal from treatment.

Immune responses against a target antigen such as PSA, PSMA, MUC1, Brachyury, CEA, or a combination thereof may be evaluated from a sample, such as a peripheral blood sample of an individual using one or more suitable tests for immune response, such as ELISpot, cytokine flow cytometry, or antibody response. A positive immune response can be determined by measuring a T-cell response. A T-cell response can be considered positive if the mean number of spots adjusted for background in six wells with antigen exceeds the number of spots in six control wells by 10 and the difference between single values of the six wells containing antigen and the six control wells is statistically significant at a level of p≤0.05 using the Student's t-test. Immunogenicity assays may occur prior to each immunization and at scheduled time points during the period of the treatment. For example, a time point for an immunogenicity assay at around week 1, 2, 3, 4, 5, 6, 7, 8, 9, 10, 11, 12, 13, 14, 15, 18, 20, 24, 30, 36, or 48 of a treatment may be scheduled even without a scheduled immunization at this time. In some cases, an individual may be considered evaluable for immune response if they receive at least a minimum number of immunizations, for example 1, 2, 3, 4, 5, 6, 7, 8, 9, or more immunizations.

In some embodiments, disease progression or clinical response determination is made according to the RECIST 1.1 criteria among patients with measurable/evaluable disease. In some embodiments, therapies using the methods and compositions as described herein affect a Complete Response (CR; disappearance of all target lesions for target lesions or disappearance of all non-target lesions and normalization of tumor marker level for non-target lesions) in an individual receiving the therapy. In some embodiments, therapies using the methods and compositions as described herein affect a Partial Response (PR; at least a 30% decrease in the sum of the LD of target lesions, taking as reference the baseline sum LD for target lesions) in an individual receiving the therapy.

In some embodiments, therapies using the methods and compositions as described herein affect a Stable Disease (SD; neither sufficient shrinkage to qualify for PR nor sufficient increase to qualify for PD, taking as reference the smallest sum LD since the treatment started for target lesions) in an individual receiving the therapy. In some embodiments, therapies using the methods and compositions described herein affect an Incomplete Response/Stable Disease (SD; persistence of one or more non-target lesion(s) or/and maintenance of tumor marker level above the normal limits for non-target lesions) in an individual receiving the therapy. In some embodiments, therapies using the methods and compositions as described herein affect a Progressive Disease (PD; at least a 20% increase in the sum of the LD of target lesions, taking as reference the smallest sum LD recorded since the treatment started or the appearance of one or more new lesions for target lesions or persistence of one or more non-target lesion(s) or/and maintenance of tumor marker level above the normal limits for non-target lesions) in an individual receiving the therapy.

### Kits

The compositions, immunotherapy or vaccines described herein may be supplied in the form of a kit. The kits of the present disclosure may further comprise instructions regarding the dosage and or administration including treatment regimen information.

In some embodiments, kits comprise the compositions and methods for providing immunotherapy or vaccines described. In some embodiment's kits may further comprise components useful in administering the kit components and instructions on how to prepare the components. In some embodiments, the kit can further comprise software for conducting monitoring patient before and after treatment with appropriate laboratory tests, or communicating results and patient data with medical staff.

The components comprising the kit may be in dry or liquid form. If they are in dry form, the kit may include a solution to solubilize the dried material. The kit may also include transfer factor in liquid or dry form. In some embodiments, if the transfer factor is in dry form, the kit includes a solution to solubilize the transfer factor. The kit may also include containers for mixing and preparing the components. The kit may also include instrument for assisting with the administration such for example needles, tubing, applicator, inhalant, syringe, pipette, forceps, measured spoon, eye dropper or any such medically approved delivery vehicle. The kits or drug delivery systems as described herein also will typically include a means for containing compositions of the present disclosure in close confinement for commercial sale and distribution.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety to the same extent as if each individual publication, patent, or patent application was specifically and individually indicated to be incorporated by reference.

Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Ad5 [E1−, E2b−]-PSA Vaccine in Mice

This example describes pre-clinical testing of the Ad5 [E1−, E2b−]-PSA vaccine in a mouse model. Studies were performed to assess the use of Ad5 [E1−, E2b−]-PSA as a cancer vaccine in a BALB/c mouse model. Ad5 [E1−, E2b−]-PSA induced potent CMI against PSA in mice. Studies were also performed to show anti-tumor activity of the vaccine in a murine model of PSA expressing cancer. These data indicate that in vivo delivery of Ad5 [E1−, E2b-]-PSA can induce PSA directed anti-tumor immunity against PSA expressing cancers.

Pre-clinical studies were performed in a BALB/c murine model to demonstrate the immunogenicity of the Ad5 [E1−, E2b−]-PSA vaccine.

Induction of CMI Responses after Ad5 [E1−, E2b−]-PSA Immunization

To assess CMI induction by flow cytometry following multiple homologous immunizations with Ad5 [E1−, E2b−]-PSA, groups of Ad5 immune BALB/c mice (n=5/group) were immunized three times SC at 1-week intervals with 10¹⁰ VP of Ad5 [E1−, E2b−]-PSA. Control mice were injected with buffer solution only. Two weeks following the last immunization, splenocytes were harvested and exposed to PSA protein and assessed for CMI responses by ELISpot for IFN-γ or IL-2 secreting splenocytes.

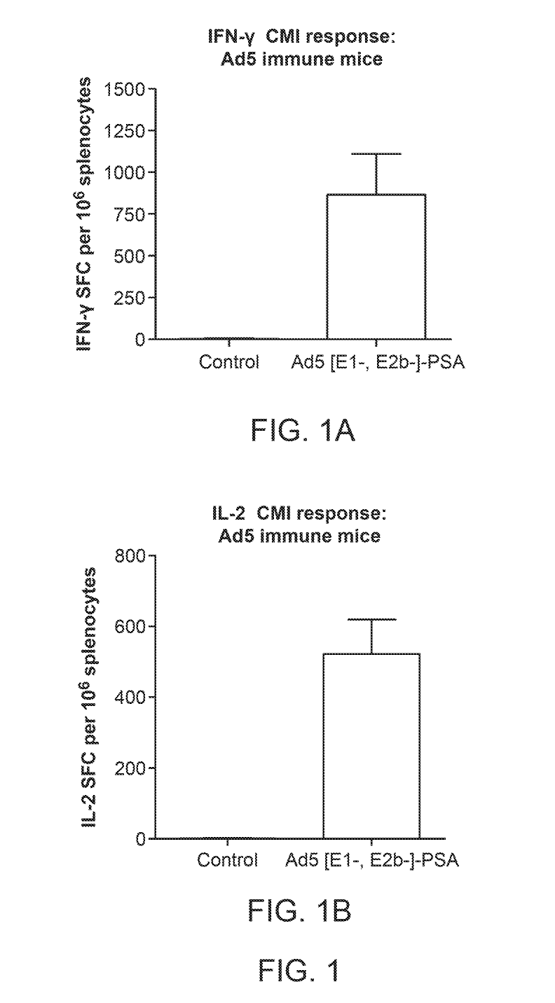

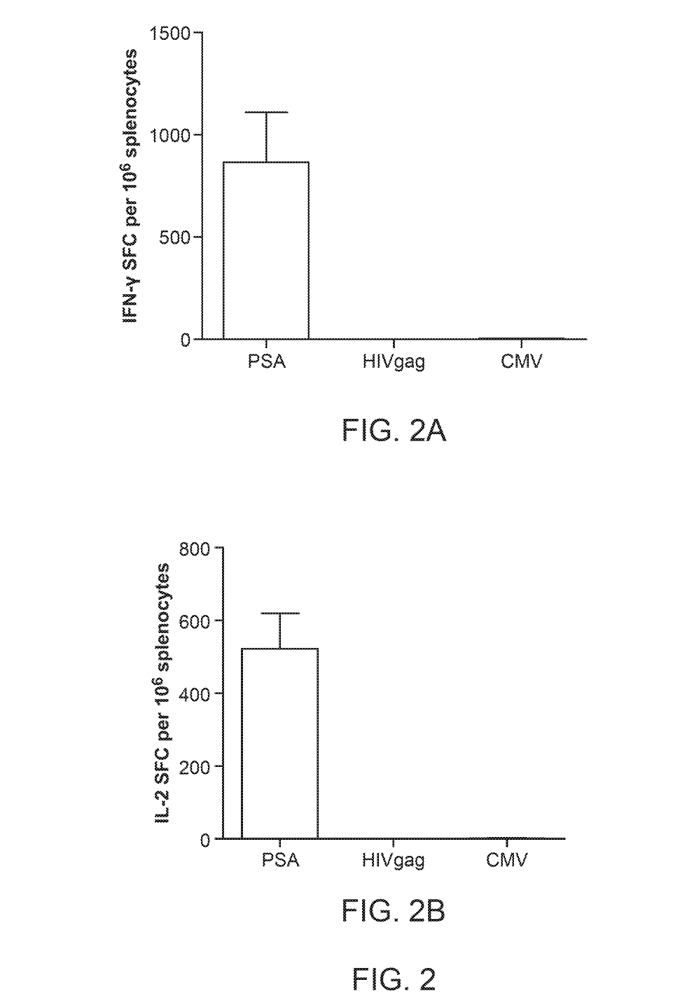

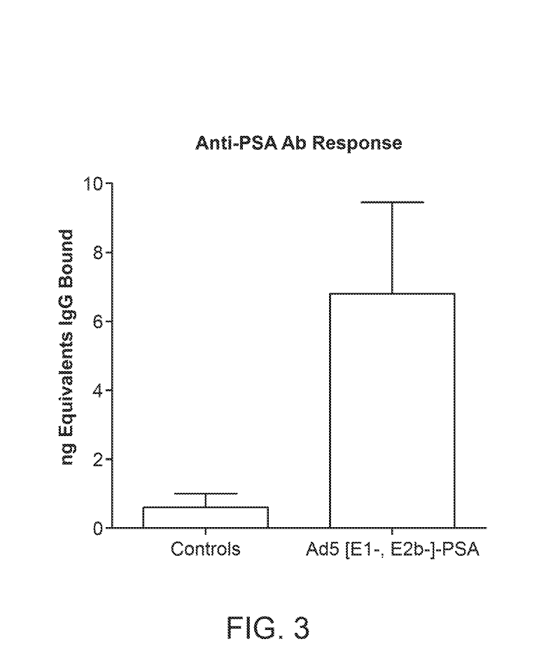

PSA directed CMI responses were induced in vaccinated but not control mice (FIGS. 1A and 1B). Specificity of the CMI responses was demonstrated in ELISpot assays using irrelevant HIV-gag or cytomegalovirus virus (CMV) antigens (FIGS. 2A and 2B). Antibody responses were also tested and PSA directed antibody responses were detected in immunized but not control mice (FIG. 3).

To determine if infected human DC could stimulate human antigen-specific T cell lines to secrete IFN-γ, specifically infected DC were incubated with antigen-specific T cell lines and tested for IFN-γ secreting activity as a measure of stimulation. Human DC were infected with Ad5 vector, incubated for 48 hours, washed, and used for stimulation of human antigen-specific T cells. As shown in TABLE 4, infection of human dendritic cells (from a HLA-A2 donor) with recombinant Ad5-PSA vectors encoding transgenes can activate PSA-specific T cell lines to produce IFN-γ.

These above results demonstrate that the Ad5 [E1−, E2b−]-PSA vaccine is effective at inducing PSA directed immune responses.

TABLE 4
Activation of PSA-specific T cell lines to produce IFN-γ

| | | Antigen-specific | T cells |
|---|---|---|---|
| Dendritic Cells | | T-PSA- | T-CEA- |
| Infected With | Peptide | (HLA-A2) | (HLA-A2) |
| Ad5 [E1, E2b]-PSA | NO | >3,000 | <0.732 |
| (20,000 MOI) | | | |
| Ad5 [E1, E2b]-PSA | NO | >3,000 | 0.84 |
| (10,000 MOI) | | | |
| Ad5 [E1, E2b]-Null | NO | 0.9 | 0.89 |
| (20,000 MOI) | | | |
| DCs | NO | 4.24 | 0.78 |
| No DCs (PSA T cells only) | NO | <0.732 | ND |
| No DCs (CEA T cells only) | NO | ND | <0.732 |

Results are expressed in picograms of IFN-γ per 5 × 10⁵ T cells/ml.
DC only = <0.732.

### Anti-Tumor Activity of the Ad5 [E1−, E2b−]-PSA Vaccine

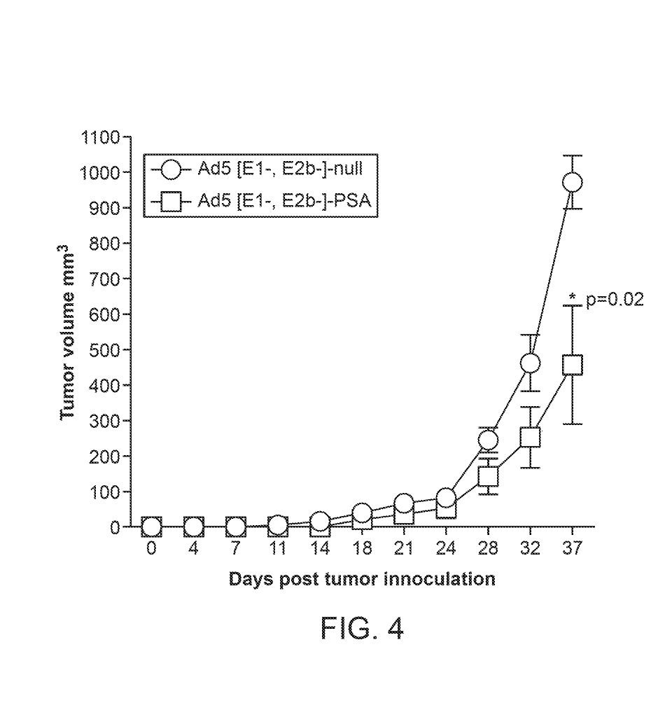

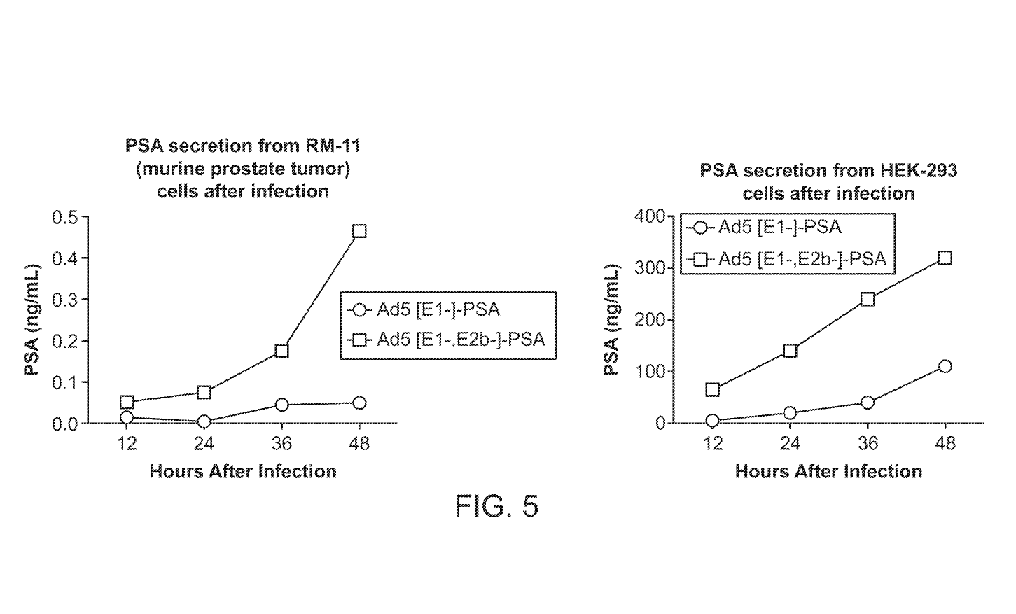

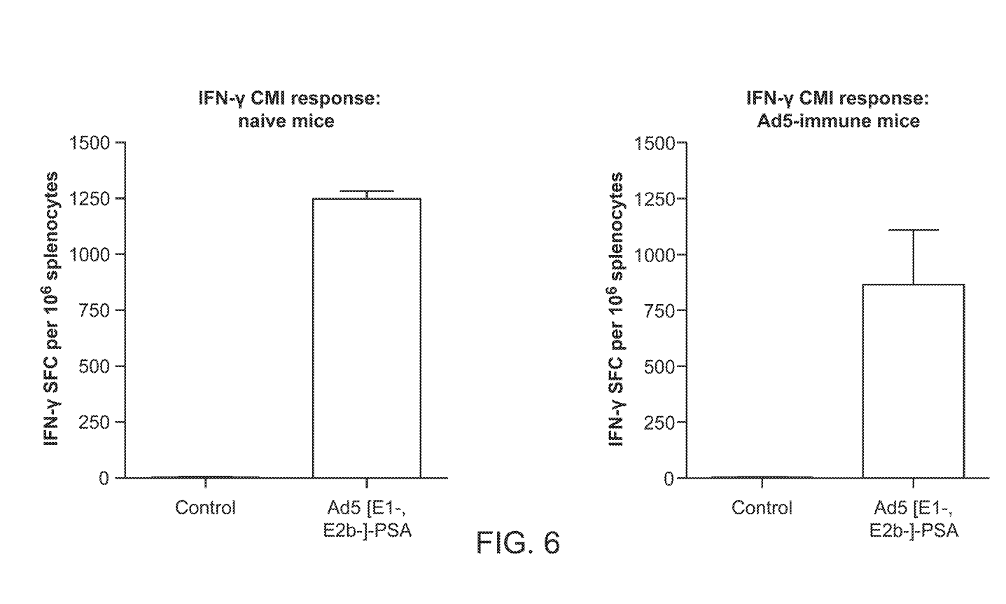

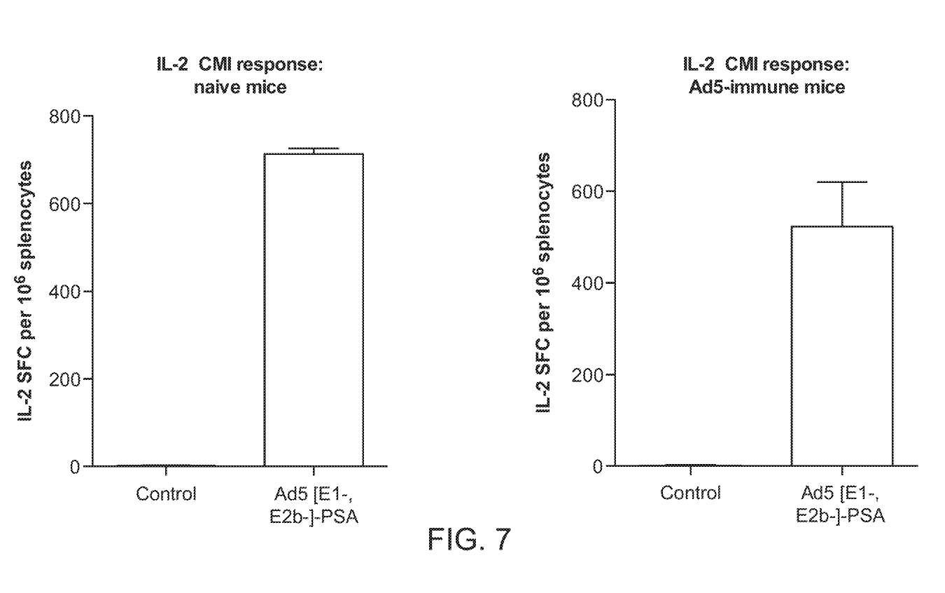

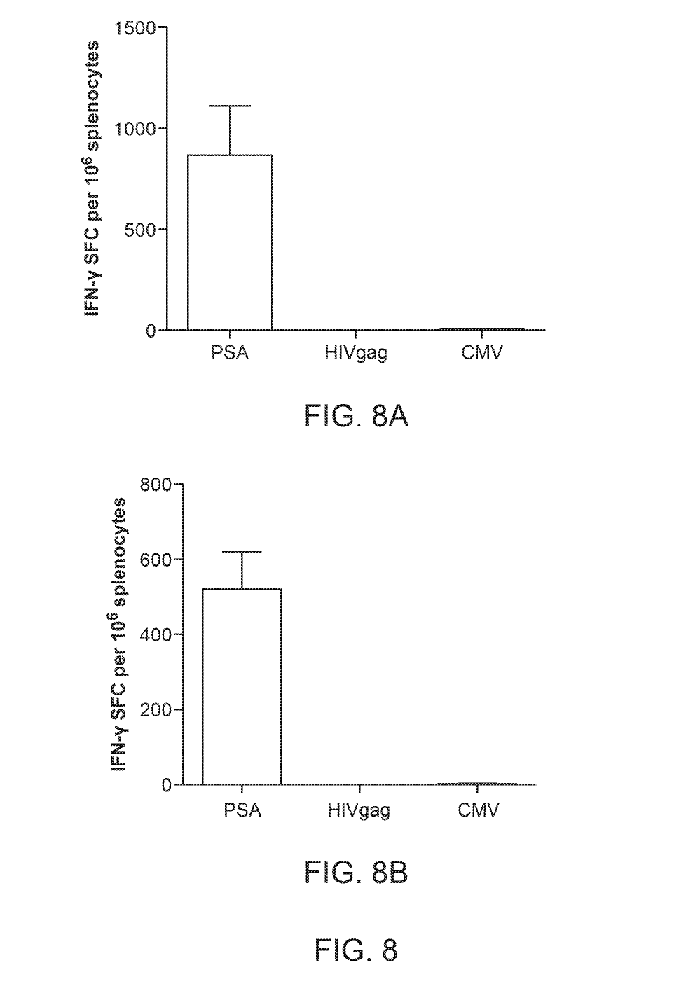

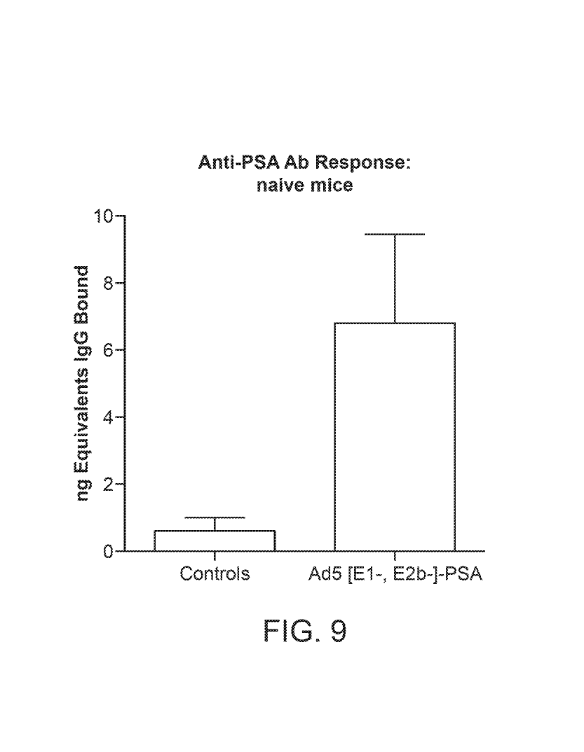

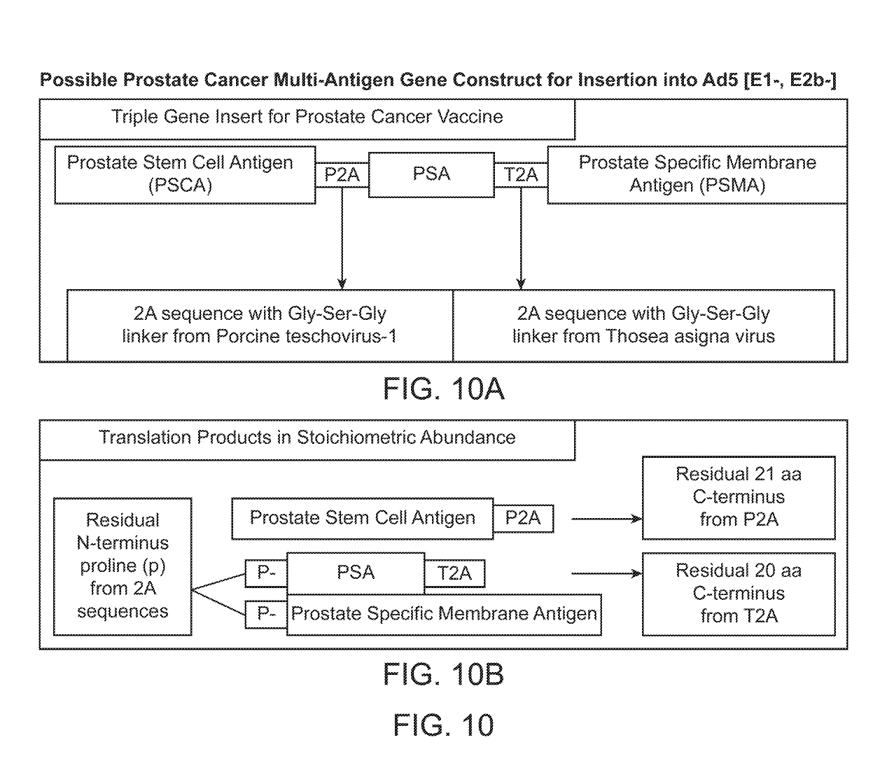

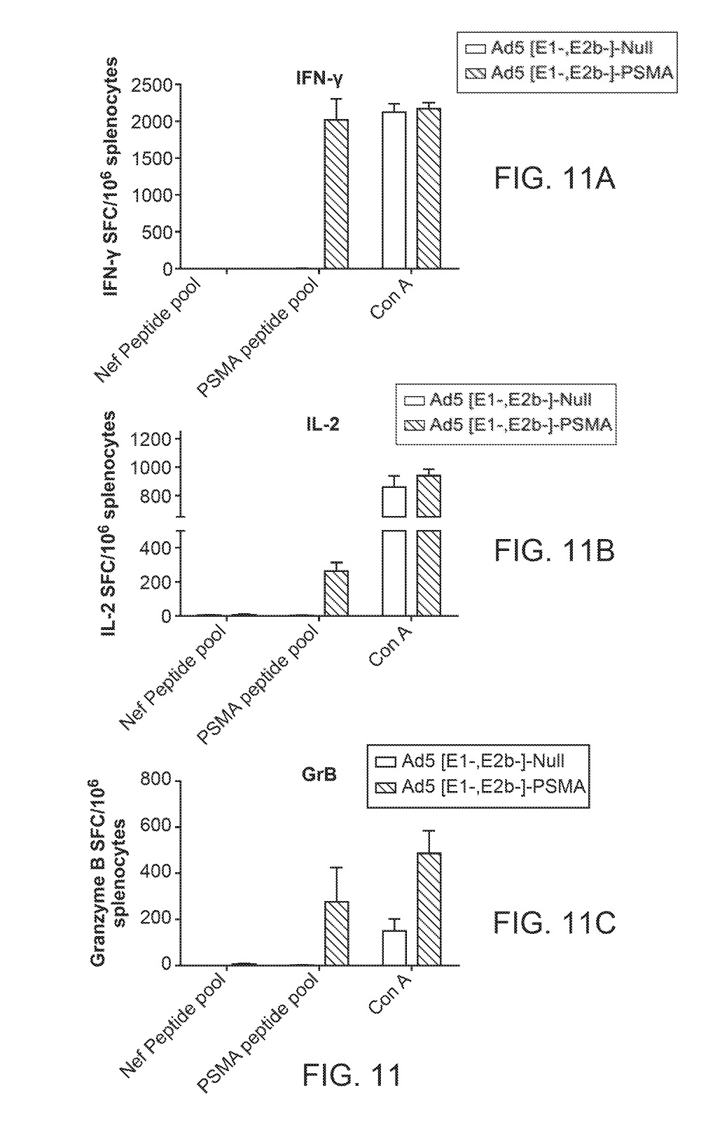

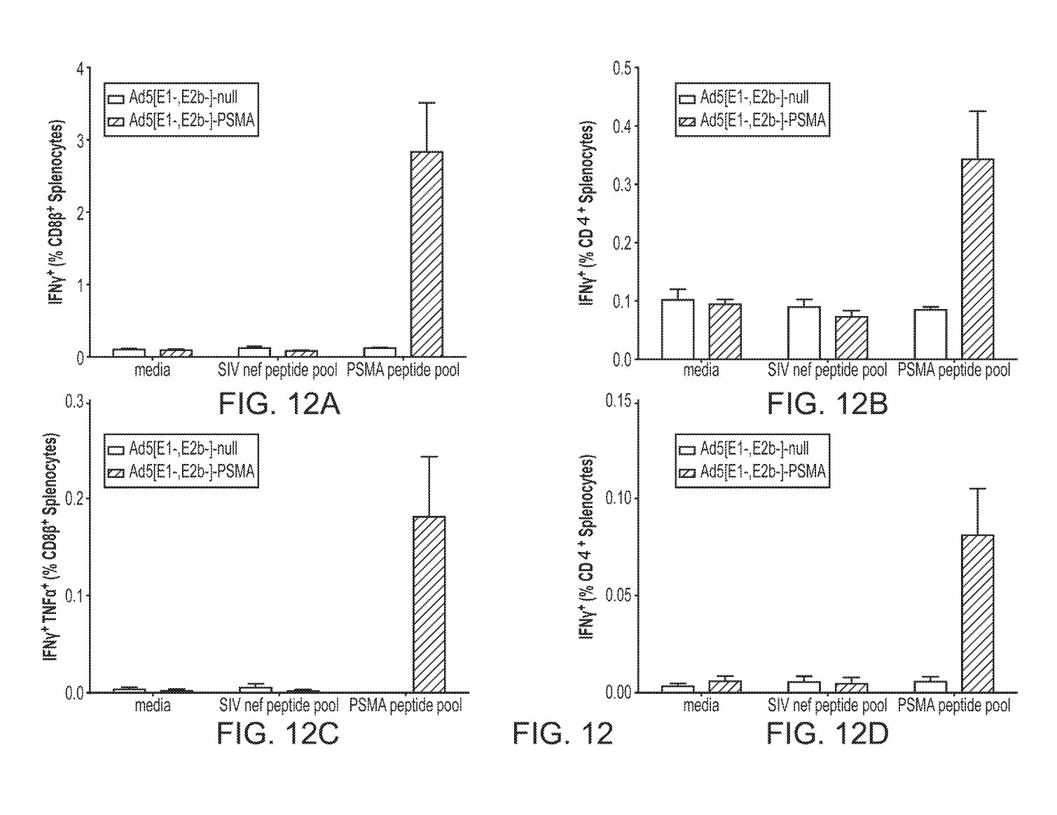

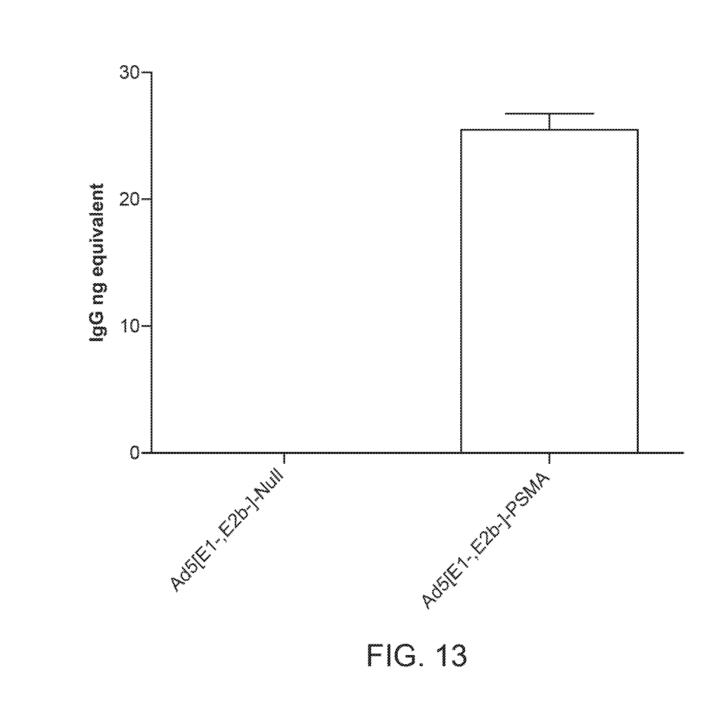

The anti-tumor activity of the Ad5 [E1−, E2b−]-PSA vaccine was tested in a murine model of PSA expressing cancer. BALB/c mice were immunized three times subcutaneously (SC) at two-week intervals with 1×10¹⁰ VP of Ad5 [E1−, E2b−]-null (empty vector controls) or 1×10¹⁰ VP of Ad5 [E1−, E2b−]-PSA vaccine. Two weeks after the last immunization (vaccination), mice were implanted with 5×10⁵ PSA expressing murine tumor cells. All mice were monitored for tumor growth and tumor volumes were calculated to determine if pre-immunization with Ad5 [E1−, E2b−]-PSA inhibited growth of tumors in immunized but not control mice. Tumor volumes were calculated according to the formula V=(tumor width²×tumor length)/2. Mice immunized with Ad5 [E1−, E2b−]-PSA experienced slower tumor growth as compared to control mice injected with Ad5 [E1−. E2b−]-null (FIG. 4). These studies indicate that the Ad5 [E1−, E2b−]-PSA vector platform has the potential to be utilized as an immunotherapeutic agent to treat PSA expressing tumors.

### Assessment of Antigen-Specific Responses by ELISPOT

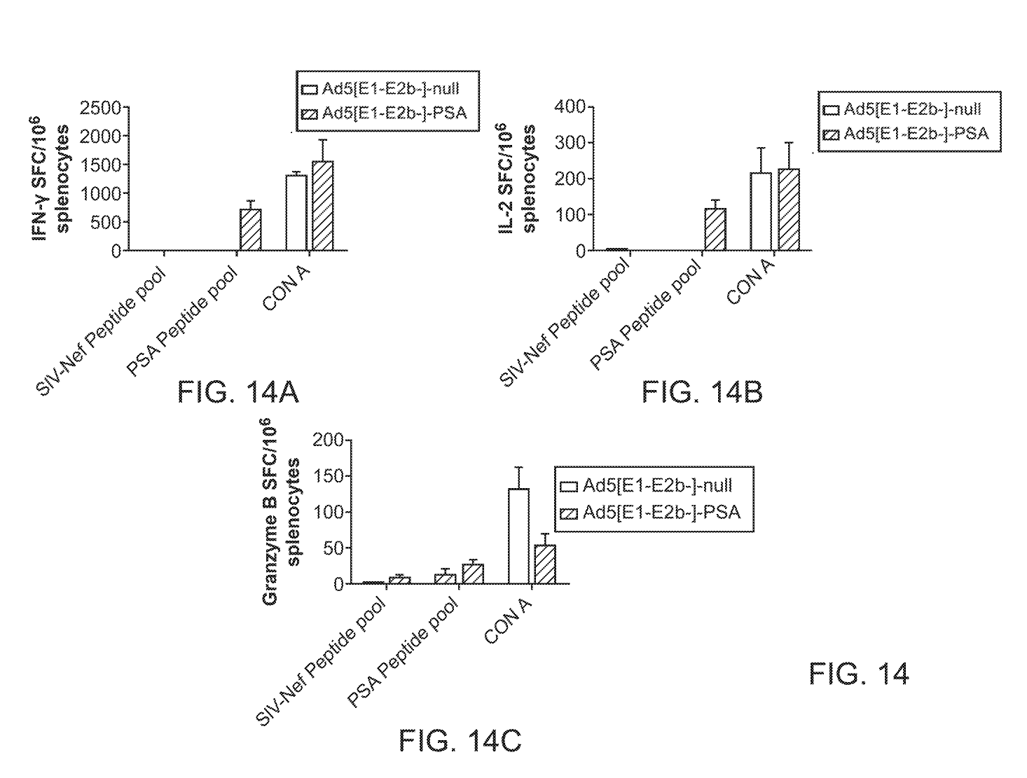

Splenocytes were collected at the end of the experiment (37 days post-tumor innoculation) and exposed ex vivo to a PSA peptide pool, a negative control (SIV-Nef peptide pool), or a positive control (Concanavalin A (Con A)). Cytokine secretion was measured after ex vivo stimulation using an ELISPOT assay, as shown in FIG. 14. Data were reported as the number of spot forming cells (SFC) per 10⁶ splenocytes and error bars show the SEM. FIG. 14A illustrates IFN-γ secreting cells after ex vivo stimulation. FIG. 14B illustrates IL-2 secreting cells after ex vivo stimulation. FIG. 14C illustrates Granzyme B secreting cells after ex vivo stimulation.

### Assessment of Antigen-Specific Responses by Intracellular Cytokine Staining and Flow Cytometry

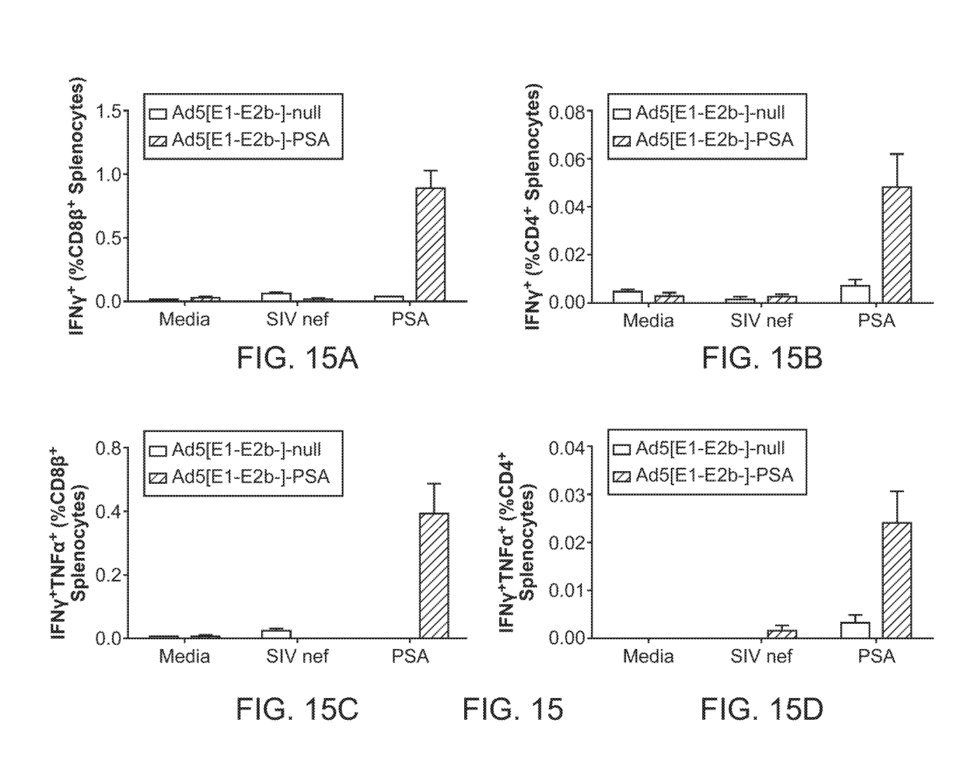

Splenocytes were collected at the end of the experiment (37 days post-tumor inoculation) and exposed ex vivo to a PSA peptide pool or a negative control antigen (media or SIV-Nef peptide pool). Cells were stained for surface markers and for intracellular cytokine secretion and analyzed by flow cytometry, as shown in FIG. 15. FIG. 15A illustrates the percent of CD8β+ splenocytes secreting IFN-γ. FIG. 15B illustrates the percent of CD4+ splenocytes secreting IFN-γ. FIG. 15C illustrates the percent of CD8β+ splenocytes secreting IFN-γ and TNF-α. FIG. 15D illustrates the percent of CD4+ splenocytes secreting IFN-γ and TNF-α.

### Assessment of Antigen Specific Antibodies Against PSA by ELISA

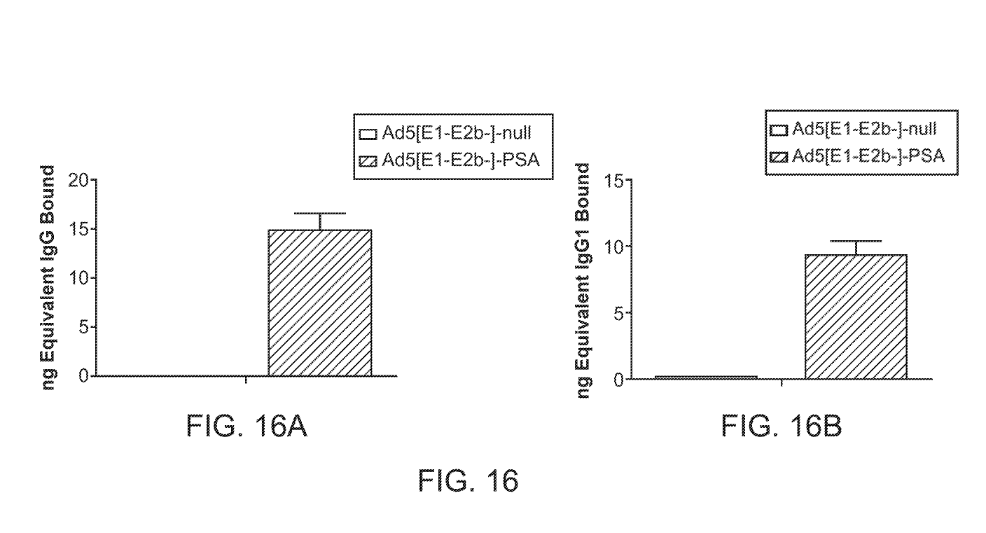

Sera was collected at the end of the experiment (37 days post-tumor inoculation) and analyzed for the presence of antibodies using an enzyme-linked immunosorbent assay (ELISA), as shown in FIG. 16. FIG. 16A illustrates the mass of IgG specific antibodies against PSA. FIG. 16B illustrates the mass of IgG1 specific antibodies against PSA.

### Assessment of Toxicity of Ad5 [E1−, E2b−]-PSA Vaccine

An extensive pre-clinical toxicology study is conducted to assess the toxicity of Ad5 [E1−, E2b−]-PSA following SC injections in BALB/c mice. Toxicity endpoints are assessed at various time points post-injection. The animals are administered up to 3 SC injections on Days 1, 22, and 43, with either vehicle control or Ad5 [E1−, E2b−]-PSA at a dose consistent with that to be used in clinical trials accounting for difference in body mass. Evaluations consist of effects on body weights, body weight gain, food consumption pathology, blood hematology analyses, blood chemistry analyses, and test on coagulation time.

In summary, Ad5 [E1−, E2b−]-PSA is a therapeutic vaccine targeting PSA that induces robust immune responses. Ad5 [E1−, E2b−]-PSA induced potent CMI against PSA in mice as assessed in ELISpot assays for IFN-γ and IL-2 secreting splenocytes. In addition, human antigen-specific T cell lines were stimulated by human DC infected with Ad5 [E1−, E2b−]-PSA.

Importantly, the Ad5 [E1−, E2b−]-PSA vaccine generated anti-tumor activity in a preclinical murine model of PSA expressing cancer.

### Example 2

### Phase I/IIa Studies of Ad5 [E1−, E2b−]-PSA Vaccine in Individuals with Advanced Prostate Cancer

This example describes a Phase I/IIa study of the Ad5 [E1−, E2b−]-PSA vaccine in individuals with advanced prostate cancer. The goal is to clinically test this therapeutic vaccine against PSA which utilizes an Ad5 vector system that overcomes barriers found with other Ad5 systems. The results of the clinical studies can establish the safety and immunogenicity of using this Ad5 [E1−, E2b−]-PSA vaccine as an immunotherapeutic agent.

The specific objective of the study is to evaluate the safety and feasibility of therapeutic immunotherapy with the Ad5 [E1−, E2b−]-PSA immunotheraputic agent in patients with advanced stage prostate cancer. Ad5 [E1−, E2b−]-PSA is designed to induce anti-tumor T cell-mediated immune responses.

Ad5 [E1−, E2b−]-PSA is an adenovirus serotype 5 (Ad5) vector that has been modified by removal of early 1 (E1), early 2b (E2b) and early 3 (E3) gene regions and insertion of the human prostate specific antigen (PSA) gene. The resulting recombinant replication-defective vector is propagated in the newly engineered, proprietary human 293 based cell line (E.C7) that supplies the E1 and E2b gene functions in trans required for vector production. No gene transfer insertion is proposed for this protocol; the product functions and remains episomal.

An open-label, dose-escalation, Phase I/IIa study is conducted with a total of up to 24 patients with PSA expressing prostate cancer. 5×10⁹, 5×10¹⁰ and 5×10¹¹ adenovirus VP dosage levels are evaluated. In Phase I, patients are enrolled into successive dosage level cohorts of 3 or 6 patients and monitored for dose-limiting toxicity (DLT). Each patient is given Ad5 [E1−, E2b−]-PSA by SC injection every 3 weeks for 3 immunizations. Assessment of DLT for dose escalation is made after all patients in a cohort have had a study visit at least 3 weeks after receiving their last dose of vaccine. Patients with a history of allergic reactions to any component of this vaccine are not included in the trial.

### Description of the Product

Ad5 [E1−, E2b−]-PSA vaccine is a clear colorless liquid filled in a 2-mL amber vial containing 1 mL of extractable vaccine. There are total of 5.0×10¹¹ total VP in 1 mL of the product. Each vial is sealed with a rubber stopper and has a white flip off seal. End user of the product flips the white plastic portion of the cap up/off with their thumb to expose the rubber stopper, and then puncture the stopper with an injection needle to withdraw the liquid. The rubber stopper is secured to the vial with an aluminum crimped seal.

Ad5 [E1−, E2b−]-PSA is characterized by high-level expression of PSA within transfected cells.

### Dosage and Administration

The dose of Ad5 [E1−, E2b−]-PSA is 5×10⁹, 5×10¹⁰, or 5×10¹¹ VP depending on which cohort the patient is enrolled. The maximum tolerated dose is determined in a dose escalation study.

The Ad5 [E1−, E2b−]-PSA vaccine is stored at ≤−20° C. Prior to injection, the appropriate vial is removed from the freezer and allowed to thaw at controlled room temperature (20-25° C., 68-77° F.) for at least 20 minutes and not more than 30 minutes, after which it is kept at 2-8° C. (35-46° F.). The vaccine is stable for at least 8 hours after removal from the freezer when kept refrigerated at 2-8° C. (35-46° F.).

The thawed vial is swirled and then, using aseptic technique, the pharmacist withdraws the appropriate volume (1 mL) from the vial using a 1 mL syringe. The vaccine is injected as soon as possible using a 1 to ½ inch, 20 to 25-gauge needle. If the vaccine cannot be injected immediately, the syringe is stored at 2-8° C. (35-46° F.).

All injections of vaccine are given as a volume of 1 mL by subcutaneous injection in the upper arm after preparation of the site with alcohol. Either arm is used for each injection.

When preparing a dose in a syringe and administering the dose, consideration is given to the volume of solution that may remain in the needle after the dose is administered, to ensure that the full dose specified in the protocol is administered.

Ad5 [E1−, E2b−]-PSA vaccine is supplied as a sterile, clear solution in a 2-mL single-dose vial. Each vial contains a single dose of vaccine provided at 5×10¹¹ VP per mL. Each vial contains a 1.3 mL total volume. The product is stored at ≤−20±10° C. until use.

Individual vials (in the desired number) of Ad5 [E1−, E2b−]-PSA are be packaged in a cardboard box and are shipped over dry ice (<−20° C.) by overnight courier with a temperature monitoring device included. Upon receipt, one inspects contents of package for any noticeable damages or defects. Unpack the shipment contents and place the cardboard box containing Ad5 [E1−, E2b−]-PSA vials into a freezer with a temperature control of <−20° C. Receiver stops the temperature monitoring device by turning off the power switch (instructions for handling and operation of temperature monitoring device are provided with the package).

### Instructions for Dose Preparation—5×10⁹ Virus Particles

From a 5.0 mL vial of 0.9% sterile saline remove 0.05 mL of fluid, leaving 4.95 mL. Then remove 0.05 mL from the vial labeled Ad5 [E1−, E2b−]-PSA and deliver this volume into the 5 mL sterile saline vial. Mix the contents by inverting the 5 mL diluted drug. Then with draw 1 mL of diluted drug and deliver to the patient by subcutaneous injection (detailed description of dose preparation is described in the packaging insert).

### Instructions for Dose Preparation—5×10¹⁰ Virus Particles

From a 5.0 mL vial of 0.9% sterile saline remove 0.5 mL of fluid, which leaves 4.5 mL. Then remove 0.5 mL from the vial labeled Ad5 [E1−, E2b−]-PSA and deliver this volume into the 5 mL sterile saline vial. Mix the contents by inverting the 5 mL diluted drug. Then with draw 1 mL of diluted drug and deliver to the patient by subcutaneous injection (detailed description of dose preparation is described in the packaging insert).

### Instructions for Dose Preparation—5×10¹¹ Virus Particles

Withdraw 1 mL of contents from vial and deliver to the patient by subcutaneous injection without any further manipulation.

### Example 3

### Production of Multi-Targeted Vaccine

This example describes production of a multi-targeted vaccine comprising more than one antigen target.

### Production of Multi-Targeted Vectors

Ad5 [E1−, E2b−]-brachyury, Ad5 [E1−, E2b−]-PSA (and/or PSMA) and Ad5 [E1−, E2b−]-MUC1 are constructed and produced. Briefly, the transgenes are sub-cloned into the E1 region of the Ad5 [E1−, E2b−] vector using a homologous recombination-based approach. The replication deficient virus is propagated in the E.C7 packaging cell line, CsCl₂ purified, and titered. Viral infectious titer is determined as plaque-forming units (PFUs) on an E.C7 cell monolayer. The VP concentration is determined by sodium dodecyl sulfate (SDS) disruption and spectrophotometry at 260 nm and 280 nm.

The sequence encoding for a human PSA antigen as in SEQ ID NO: 1 or SEQ ID NO: 35 is constructed and subsequently cloned into the Ad5 vector to generate the Ad5 [E1−, E2b−]-PSA construct. Similarly, the sequence encoding for a human PSMA antigen as in SEQ ID NO: 11 is constructed and subsequently cloned into the Ad5 vector to generate the Ad5 [E1−, E2b−]-PSMA construct.

The sequence encoding for the human Brachyury protein (T, NM_003181.3) is modified by introducing the enhancer T-cell HLA-A2 epitope (WLLPGTSTV; SEQ ID NO: 7) and removal of a 25 amino acid fragment involved in DNA binding. The resulting construct is subsequently subcloned into the Ad5 vector to generate the Ad5 [E1−, E2b−]-Brachyury construct.

The MUC1 molecule consisted of two regions: the N-terminus (MUC1-n), which is the large extracellular domain of MUC1, and the C-terminus (MUC1-c), which has three regions: a small extracellular domain, a single transmembrane domain, and a cytoplasmic tail. The cytoplasmic tail contained sites for interaction with signaling proteins and acts as an oncogene and a driver of cancer motility, invasiveness and metastasis. For construction of the Ad5 [E1−, E2b−]-MUC1, the entire MUC1 transgene, including eight agonist epitopes, will be subcloned into the Ad5 vector. The agonist epitopes included in the Ad5 [E1−, E2b−]-MUC1 vector bind to HLA-A2 (epitope P93L in the N-terminus, VIA and V2A in the VNTR region, and CIA, C2A and C3A in the C-terminus), HLA-A3 (epitope C5A), and HLA-A24 (epitope C6A in the C-terminus).

The Tri-Ad5 vaccine is produced by combining of 10¹⁰ VP of Ad5 [E1−, E2b−]-Brachyury, Ad5 [E1−, E2b−]-PSA (or alternatively Ad5 [E1−, E2b−]-PSMA) and Ad5 [E1−, E2b−]-MUC1 at a ratio of 1:1:1 (3×10¹⁰ VP total).

### GLP Production of Multi-Targeted Vaccine

The following shows the production of clinical-grade multi-target vaccine using good laboratory practice (GLP) standards. The Ad5 [E1−, E2b−]-PSA (and/or PSMA), Ad5 [E1−, E2b−]-MUC1 and the Ad5 [E1−, E2b−]-Brachyury products can be produced in a 5 L Cell Bioreactor.

Briefly, vials of the E.C7 manufacturing cell line are thawed, transferred into a T225 flask, and initially cultured at 37° C. in 5% CO₂ in DMEM containing 10% FBS/4 mM L-glutamine. After expansion, the E.C7 cells are expanded using 10-layered CellSTACKS (CS-10) and transitioned to FreeStyle serum-free medium (SFM). The E.C7 cells are cultured in SFM for 24 hours at 37° C. in 5% CO₂ to a target density of 5×10⁵ cells/mL in the Cell Bioreactor. The E.C7 cells will then be infected with the Ad5 [E1−, E2b−]-PSA, Ad5 [E1−, E2b−]-MUC1 or Ad5 [E1−, E2b−]-Brachyury, respectively, and cultured for 48 hours.

Mid-stream processing is performed 30 minutes before harvest, and Benzonase nuclease will be added to the culture to promote better cell pelleting for concentration. After pelleting by centrifugation, the supernatant is discarded and the pellets re-suspended in Lysis Buffer containing 1% Polysorbate-20 for 90 minutes at room temperature. The lysate will then be treated with Benzonase and the reaction quenched by addition of 5M NaCl. The slurry will be centrifuged and the pellet discarded. The lysate will be clarified by filtration and subjected to a two-column ion exchange procedure.

To purify the vaccine products, a two-column anion exchange procedure is performed. A first column is packed with Q Sepharose XL resin, sanitized, and equilibrated with loading buffer. The clarified lysate is loaded onto the column and washed with loading buffer. The vaccine product is eluted and the main elution peak (eluate) containing the Ad5 [E1−, E2b−]-PSA (and/or PSMA), Ad5 [E1−, E2b−]-MUC1 or Ad5 [E1−, E2b−]-Brachyury carried forward to the next step. A second column is packed with Source 15Q resin, sanitized, and equilibrated with loading buffer. The eluate from the first anion exchange column is loaded onto the second column and the vaccine product eluted with a gradient starting at 100% Buffer A (20 mM Tris, 1 mM MgCl₂, pH 8.0) running to 50% Buffer B (20 mM Tris, 1 mM MgCl₂, 2M NaCl, pH 8.0). The elution peaks containing the Ad5 [E1−, E2b−]-PSA (and/or PSMA), Ad5 [E1−, E2b−]-MUC1 or Ad5 [E1−, E2b−]-Brachyury are collected and stored overnight at 2-8° C. The peak elution fractions are processed through a tangential flow filtration (TFF) system for concentration and diafiltration against formulation buffer (20 mM Tris, 25 mM NaCl, 2.5% (v/v) glycerol, pH 8.0). After processing, the final vaccine products are sterile filtered, dispensed into aliquots, and stored at ≤−60° C. A highly purified product approaching 100% purity is typically produced and similar results for these products are predicted.

The concentration and total number of VP product produced are determined spectrophotometrically. Product purity is assessed by HPLC. Infectious activity is determined by performing an Ad5 hexon-staining assay for infectious particles using kits.

Western blots are performed using lysates from vector transfected A549 cells to verify PSA, PSMA, MUC1 or Brachyury expression. Quality control tests are performed to determine that the final vaccine products are mycoplasma-free, have no microbial bioburden, and exhibit endotoxin levels less than 2.5 endotoxin units (EU) per mL. To confirm immunogenicity, the individual vectors are tested in mice as described below (Example 4).

### Example 4

### Immunogenicity of Multi-Targeted PSA (and/or PSMA), MUC1, Brachyury Viral Vector

This example describes immunogenicity results using a multi-targeted vaccine against PSA (and/or PSMA), MUC1 and T (i.e., Brachyury). Each viral vector product is tested for purity, infectivity, and antigen expression, as described herein and each passed these criteria.

### Vaccination and Splenocyte Preparation

Female C57BL/6 mice (n=5) are injected SC with 10¹⁰ VP of Ad5 [E1−, E2b−]-Brachyury or Ad5 [E1−, E2b−]-PSA (and/or PSMA) or Ad5 [E1−, E2b−]-MUC1 or a combination of 10¹⁰ VP of all three viruses at a ratio of 1:1:1 (Tri-Ad5 with PSA and/or PSMA, MUC1, and Brachyury). Control mice are injected with 3×10¹⁰ VP of Ad-null (no transgene insert). Doses are administered in 25 μl of injection buffer (20 mM HEPES with 3% sucrose) and mice are vaccinated three times at 14-day intervals. Fourteen days after the final injection spleens and sera are collected. Sera are frozen at −20° C. Splenocyte suspensions are generated by gently crushing the spleens through a 70 μM nylon cell strainer (BD Falcon, San Jose, Calif.). Red cells are removed by the addition of red cell lysis buffer (Sigma-Aldrich, St. Louis, Mo.) and the splenocytes are washed twice and resuspended in R10 (RPMI 1640 supplemented with L-glutamine (2 mM), HEPES (20 mM), penicillin 100 U/ml and streptomycin 100 μg/ml, and 10% fetal bovine serum. Splenocytes are assayed for cytokine production by ELISPOT and flow cytometry.

### Immunogenicity Studies:

Immunization with Ad5 [E1−, E2b−] vectors is dose-dependent and 1×10¹⁰ VP per dose is used. Groups (N=5) of C57B1/6 mice are used.

In this study, C57B1/6 mice are injected subcutaneously 3 times at one-week intervals or 2-week intervals with tri-immunization comprising 1×10¹⁰ virus particles (VP) Ad5 [E1−, E2b−]-null (empty vector controls) or with 1×10¹⁰ VP containing a 1:1:1 mixture of Ad5 [E1−, E2b−]-PSA (and/or PSMA), Ad5 [E1−, E2b−]-MUC1, and Ad5 [E1−, E2b−]-Brachyury.

Two weeks after the last immunization CMI activity is determined employing ELISpot assays for IFN-γ secreting cells (SFC) after exposure of splenocytes to PSA, MUC1, or Brachyury peptide pools, respectively.

Significant CMI responses to the multi-targeted vectors are detected in immunized mice. Flow cytometry utilizing intracellular cytokine staining is performed on spleen cells after exposure to PSA and/or PSMA peptides to assess the quantity of activated CD4+ and CD8+ T-cells.

Briefly, CMI responses against PSA, PSMA, MUC1, and Brachyury as assessed by ELISpot assays for IFN-γ secreting splenocytes (SFC) are detected in multi-targeted immunized mice but not control mice (injected with Ad5-Null empty vector). Specificity of the ELISpot assay responses is confirmed by lack of reactivity to irrelevant SIV-nef or SIV-vif peptide antigens. A positive control includes cells exposed to concanavalin A (Con A).

### Anti-Tumor Immunotherapy Studies:

Studies are conducted to test the anti-tumor capability of Ad5 [E1−, E2b−]-based tri-vaccines (Tri-Ad5, i.e., Ad5 [E1−, E2b−]-PSA (and/or PSMA), Ad5 [E1−, E2b−]-MUC1, and/or Ad5 [E1−, E2b−]-Brachyury) in immunotherapy studies in mice with established PSA, MUC1, or Brachyury expressing tumors, respectively. In this study the anti-tumor activity of the individual components of the Ad5 [E1−, E2b−]-based tri-vaccine are assessed.

For in vivo tumor treatment studies, groups (n=7) of C57Bl/6 mice are injected subcutaneously in the right flank with 5×10⁵ PSA (and/or PSMA), MUC1, and/or Brachyury expressing murine tumor cells. After palpable tumors are detected, mice are treated by 3 subcutaneous injections at a weekly interval with 1×10¹⁰ VP each of Ad5 [E1−, E2b−]-null (no transgene, e.g., empty vector), Ad5 [E1−, E2b−]-PSA (and/or PSMA), Ad5 [E1−, E2b−]-MUC1, and/or Ad5 [E1−, E2b−]-Brachyury, respectively. Control mice are injected with 3×10¹⁰ VP of Adeno-null. Tumor volumes are calculated and tumor growth curves are plotted. 7-10 mice/group are sufficient for statistical evaluation of treatment. Tumor studies are terminated when tumors reached 1500 m³ or became severely ulcerated.

Larger numbers of mice are treated to show significant anti-tumor activity and to combine immunotherapy with immune pathway checkpoint modulators, such as anti-checkpoint inhibitor antibodies, to determine if anti-tumor activity is enhanced.

### Example 5

### PSA Antibody Activity Following Vaccination

This example describes induction of PSA antibody activity following vaccination. PSA antibody activity is assessed from sera of mice vaccinated with Ad5 [E1−, E2b−]-PSA/B7-1/ICAM-1/LFA-3. PSA IgG levels as determined by ELISA in mice that are vaccinated three times with Ad5 [E1−, E2b−]-PSA/B7-1/ICAM-1/LFA-3.

Complement-dependent cellular cytotoxicity (CDCC) against PSA-expressing tumor cells in the same groups of mice is demonstrated by test subjects. Cytotoxic activity is observed in vaccinated mice but not in control mice or in cells exposed to the complement only.

### Example 6

### Ad5 [E1−, E2b−]-PSA/B7-1/ICAM-1/LFA-3 Combination Immunotherapy Clinical Trial

This example describes a clinical trial of Ad5 [E1−, E2b−]-PSA/B7-1/ICAM-1/LFA-3 as a combination therapy. A clinical trial employs a combination of an Ad5 [E1−, E2b−]-PSA/B7-1/ICAM-1/LFA-3 vaccine and anti-PDL1 antibody for immunotherapy in prostate cancer patients. The phase I portion of the study determines the safety of immunization with Ad5 [E1−, E2B−]-PSA/B7-1/ICAM-1/LFA-3 in patients with prostate cancer. The Phase II portion of the study evaluates patient immune responses to the immunizations and the clinical feasibility of treating prostate cancer with an Ad5 [E1−, E2b−]-PSA/B7-1/ICAM-1/LFA-3 vaccine in combination with an anti-PDL1 antibody.

The study population consists of patients with a histologically confirmed diagnosis of prostate cancer that is PSA positive. The safety of three dosage levels of Ad5 [E1−, E2B−]-PSA/B7-1/ICAM-1/LFA-3 vaccine (phase I component), and the safety and suitability of using an Ad5 [E1−, E2B−]-PSA/B7-1/ICAM-1/LFA-3 vaccine in combination with an anti-PDL1 antibody for the treatment of prostate cancer (phase II component) are determined by the study.

The phase I study drug is Ad5 [E1−, E2B−]-PSA/B7-1/ICAM-1/LFA-3 given by subcutaneous (SC) injection every 3 weeks for 3 immunizations. The phase II study drug is Ad5 [E1−, E2B−]-PSA/B7-1/ICAM-1/LFA-3 in combination with an anti-PDL1 antibody given by subcutaneous (SC) injection every 3 weeks for 3 immunizations. Safety is evaluated in each cohort at least 3 weeks after the last patient in the previous cohort has received their first injection. A dosing scheme is considered safe if <33% of patients treated at a dosage level experience DLT (e.g., 0 of 3, ≤1 of 6, ≤3 of 12 or ≤5 of 18 patients).

### Example 7

### Ad5 [E1−, E2b−]-PSA/B7-1/ICAM-1/LFA-3, Ad5 [E1−, E2b−]-PSMA/B7-1/ICAM-1/LFA-3, Ad5 [E1−, E2b−]-MUC1/B7-1/ICAM-1/LFA-3, Ad5 [E1−, E2b−]-Brachyury/B7-1/ICAM-1/LFA-3, and Anti-PDL1 Antibody Combination Immunotherapy Clinical Trial

This example describes a clinical trial of Ad5 [E1−, E2b−]-PSA/B7-1/ICAM-1/LFA-3, Ad5 [E1−, E2b−]-PSMA/B7-1/ICAM-1/LFA-3, Ad5 [E1−, E2b−]-MUC1/B7-1/ICAM-1/LFA-3, Ad5 [E1−, E2b−]-Brachyury/B7-1/ICAM-1/LFA-3, and an anti-PDL1 antibody as a combination therapy. A clinical trial employs a combination of: an Ad5 [E1−, E2b−]-PSA/B7-1/ICAM-1/LFA-3 vaccine, an Ad5 [E1−, E2b−]-PSMA/B7-1/ICAM-1/LFA-3 vaccine, an Ad5 [E1−, E2b−]-MUC1/B7-1/ICAM-1/LFA-3 vaccine, an Ad5 [E1−, E2b−]-Brachyury/B7-1/ICAM-1/LFA-3 vaccine, and anti-PDL1 antibody for immunotherapy in advanced stage PSA-expressing prostate cancer patients. The phase I portion of the study determines the safety of immunization with Ad5 [E1−, E2b−]-PSA/B7-1/ICAM-1/LFA-3, an Ad5 [E1−, E2b-]-PSMA/B7-1/ICAM-1/LFA-3 vaccine, an Ad5 [E1−, E2b−]-MUC1/B7-1/ICAM-1/LFA-3, an Ad5 [E1−, E2b−]-Brachyury/B7-1/ICAM-1/LFA-3 vaccines in patients with prostate cancer. The Phase II portion of the study evaluates patient immune responses to the immunizations and the clinical feasibility of treating prostate cancer with Ad5 [E1−, E2b−]-PSA/B7-1/ICAM-1/LFA-3, an Ad5 [E1−, E2b−]-PSMA/B7-1/ICAM-1/LFA-3 vaccine, an Ad5 [E1−, E2b−]-MUC1/B7-1/ICAM-1/LFA-3, an Ad5 [E1−, E2b−]-Brachyury/B7-1/ICAM-1/LFA-3 vaccines in combination with an anti-PDL1 antibody.

The study population consists of patients with a histologically confirmed diagnosis of prostate cancer that is PSA positive. The safety of three dosage levels of Ad5 [E1−, E2b−]-PSA/B7-1/ICAM-1/LFA-3, an Ad5 [E1−, E2b−]-PSMA/B7-1/ICAM-1/LFA-3 vaccine, an Ad5 [E1−, E2b−]-MUC1/B7-1/ICAM-1/LFA-3, an Ad5 [E1−, E2b−]-Brachyury/B7-1/ICAM-1/LFA-3 vaccines (phase I component), and the safety and suitability of using Ad5 [E1−, E2b-]-PSA/B7-1/ICAM-1/LFA-3, an Ad5 [E1−, E2b−]-PSMA/B7-1/ICAM-1/LFA-3 vaccine, an Ad5 [E1−, E2b−]-MUC1/B7-1/ICAM-1/LFA-3, an Ad5 [E1−, E2b−]-Brachyury/B7-1/ICAM-1/LFA-3 vaccines in combination with an anti-PDL1 antibody for the treatment of prostate cancer (phase II component) are determined by the study.

The phase I study drug is a combination of Ad5 [E1−, E2b−]-PSA/B7-1/ICAM-1/LFA-3, an Ad5 [E1−, E2b−]-PSMA/B7-1/ICAM-1/LFA-3 vaccine, an Ad5 [E1−, E2b−]-MUC1/B7-1/ICAM-1/LFA-3, an Ad5 [E1−, E2b−]-Brachyury/B7-1/ICAM-1/LFA-3 vaccines given by subcutaneous (SC) injection every 3 weeks for 3 immunizations. The phase II study drug is Ad5 [E1−, E2b−]-PSA/B7-1/ICAM-1/LFA-3, an Ad5 [E1−, E2b−]-PSMA/B7-1/ICAM-1/LFA-3 vaccine, an Ad5 [E1−, E2b−]-MUC1/B7-1/ICAM-1/LFA-3, an Ad5 [E1−, E2b−]-Brachyury/B7-1/ICAM-1/LFA-3 vaccines in combination with an anti-PDL1 antibody given by subcutaneous (SC) injection every 3 weeks for 3 immunizations. Safety is evaluated in each cohort at least 3 weeks after the last patient in the previous cohort has received their first injection. A dosing scheme is considered safe if <33% of patients treated at a dosage level experience DLT (e.g., 0 of 3, ≤1 of 6, ≤3 of 12 or ≤5 of 18 patients).

### Example 8

### Treatment of Cancer with Ad5 [E1−, E2b−]-PSA and/or Ad5 [E1−, E2b−]-PSMA

This example describes treatment of cancer, including a PSA-expressing and/or PSMA-expressing cancer, in a subject in need thereof. Ad5 [E1−, E2b−] vectors encoding for PSA or PSMA are administered to a subject in need thereof at a dose of 1×10⁹-5×10¹¹ virus particles (VPs) subcutaneously. Vaccines are administered a total of 3-times and each vaccination is separated by a 3 week interval. Thereafter, a booster injection is given every two months (bi-monthly). The subject is any animal, for example a mammal, such as a mouse, human, or non-human primate. Upon administration of the vaccine, the cellular and humoral responses are initiated against the PSA-expressing or PSMA expressing cancer and the cancer is eliminated.

### Example 9

### Combination Treatment of Cancer with Ad5 [E1−, E2b−]-PSA and/or Ad5 [E1−, E2b−]-PSMA and Co-Stimulatory Molecules

This example describes combination treatment of cancer, including a PSA-expressing and/or PSMA-expressing cancer, in a subject in need thereof. Ad5 [E1−, E2b−] vectors encoding for PSA or PSMA are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3 week interval. Thereafter, bi-monthly booster injections are administered. The co-stimulatory molecule is B7-1, ICAM-1, or LFA-3. The subject is any animal, for example a mammal, such as a mouse, human, or non-human primate. Upon administration of the vaccine and co-stimulatory molecule, the cellular and humoral responses are initiated against the PSA-expressing or PSMA-expressing cancer and the cancer is eliminated.

### Example 10

### Combination Treatment of Cancer with Ad5 [E1−, E2b−]-PSA and/or Ad5 [E1−, E2b−]-PSMA and Checkpoint Inhibitors

This example describes treatment of cancer, including a PSA-expressing and/or PSMA-expressing cancer, in a subject in need thereof. Ad5 [E1−, E2b−] vectors encoding for PSA and/or PSMA are administered to a subject in need thereof at a dose of 1×10⁹-5×10¹¹ virus particles (VPs) subcutaneously in combination with a checkpoint inhibitor. Vaccines are administered a total of 3 times and each vaccination is separated by a 3-week interval. Thereafter, bi-monthly booster injections are administered. The checkpoint inhibitor is an anti-PDL1 antibody, such as Avelumab. Avelumab is dosed and administered as per package insert labeling at 10 mg/kg. The subject is any animal, for example a mammal, such as a mouse, human, or non-human primate. Upon administration of the vaccine and the checkpoint inhibitor, the cellular and humoral responses are initiated against the PSA-expressing or PSMA-expressing cancer and the cancer is eliminated.

### Example 11

### Combination Treatment of Cancer with Ad5 [E1−, E2b−]-PSA and/or Ad5 [E1−, E2b−]-PSMA and Engineered NK Cells

This example describes combination treatment of cancer, including a PSA-expressing and/or PSMA-expressing cancer, in a subject in need thereof. Ad5 [E1−, E2b−] vectors encoding for PSA and/or PSMA are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3-week interval. Thereafter, bi-monthly booster injections are administered. Subjects are additionally administered engineered NK cells, specifically activated NK cells (aNK cells). aNK cells are infused on days −2, 12, 26, and 40 at a dose of 2×10⁹ cells per treatment. Subjects in need thereof have CEA-expressing cancer cells, such as colorectal cancer. Subjects are any mammal, such as a human or a non-human primate.

### Example 12

### Combination Treatment of Cancer with Ad5 [E1−, E2b−]-PSA and/or Ad5 [E1−, E2b−]-PSMA and ALT-803

This example describes combination treatment of cancer, including a PSA-expressing and/or PSMA-expressing cancer, in a subject in need thereof. Ad5 [E1−, E2b−] vectors encoding for PSA and/or PSMA are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3-week interval. Thereafter, bi-monthly booster injections are administered. Subjects are also administered a super-agonist/super-agonist complex, such as ALT-803, at a dose of 10 μg/kg SC on weeks 1, 2, 4, 5, 7, and 8, respectively. Subjects in need thereof have CEA-expressing cancer cells, such as colorectal cancer. Subjects are any mammal, such as a human or a non-human animal.

### Example 13

### Combination Treatment of Cancer with Ad5 [E1−, E2b−]-PSA and/or Ad5 [E1−, E2b−]-PSMA and Low Dose Chemotherapy

This example describes combination treatment of cancer, including a PSA-expressing and/or PSMA-expressing cancer, in a subject in need thereof. Ad5 [E1−, E2b−] vectors encoding for PSA and/or PSMA are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3-week interval. Thereafter, bi-monthly booster injections are administered.

Subjects are also administered low dose chemotherapy. The chemotherapy is cyclophosphamide. The chemotherapy is administered at a dose that is lower than the clinical standard of care dosing. For example, the chemotherapy is administered at 50 mg twice a day (BID) on days 1-5 and 8-12 every 2 weeks for a total of 8 weeks. Subjects in need thereof have CEA-expressing cancer cells, such as colorectal cancer. Subjects are any mammal, such as a human or a non-human animal.

### Example 14

### Combination Treatment of Cancer with Ad5 [E1−, E2b−]-PSA and/or Ad5 [E1−, E2b−]-PSMA and Low Dose Radiation

This example describes combination treatment of cancer, including a PSA-expressing and/or PSMA-expressing cancer, in a subject in need thereof. Ad5 [E1−, E2b−] vectors encoding for PSA and/or PSMA are administered to a subject in need thereof at a dose of 1×10⁹-5×10¹¹ virus particles (VPs) subcutaneously in combination with a costimulatory molecule. Vaccines are administered a total of 3 times and each vaccination is separated by a 3-week interval. Thereafter, bi-monthly booster injections are administered.

Subjects are also administered low dose radiation. The low dose radiation is administered at a dose that is lower than the clinical standard of care dosing. Concurrent sterotactic body radiotherapy (SBRT) at 8 Gy is given on day 8, 22, 36, 50 (every 2 weeks for 4 doses). Radiation is administered to all feasible tumor sites using SBRT. Subjects in need thereof have CEA-expressing cancer cells, such as colorectal cancer. Subjects are any mammal, such as a human or a non-human animal.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising a replication-defective virus vector comprising a nucleic acid sequence encoding a prostate specific antigen (PSA) and/or a nucleic acid sequence encoding prostate-specific membrane antigen (PSMA), wherein the PSA has an amino acid sequence at least 80%, at least 85%, at least 90%, at least 92%, at least 95%, at least 97%, or at least 99% identical with SEQ ID NO: 1 or SEQ ID NO: 34 or the PSMA has an amino acid sequence at least 80% identical with SEQ ID NO: 11.

2. The composition of claim 1, wherein the vector comprises a nucleic acid sequence encoding a PSA having an amino acid sequence at least 80%, at least 85%, at least 90%, at least 92%, at least 95%, at least 97%, or at least 99% identical with SEQ ID NO: 35 or the nucleic acid sequence encoding PSA has at least 80%, at least 85%, at least 90%, at least 92%, at least 95%, at least 97%, or at least 99% identical with SEQ ID NO: 2.

3. The composition of claim 1, wherein the vector comprises a nucleic acid sequence encoding a PSMA having an amino acid sequence at least 80%, at least 85%, at least 90%, at least 92%, at least 95%, at least 97%, or at least 99% identical with SEQ ID NO: 36.

4. The composition of claim 1, further comprising a second replication-defective virus vector comprising a second nucleic acid sequence encoding a Brachyury antigen, a third replication-defective virus vector comprising a third nucleic acid sequence encoding a MUC1 antigen, or a combination thereof.

5. (canceled)

6. (canceled)

7. (canceled)

8. The composition of claim 4, wherein the second replication-defective vector comprises a nucleotide sequence at least 80%, at least 85%, at least 90%, at least 92%, at least 95%, at least 97%, or at least 99% identical with SEQ ID NO: 3, SEQ ID NO: 4, positions 13 to 1242 of SEQ ID NO: 4, SEQ ID NO: 42.

9. The composition of claim 4, wherein the second replication-defective vector comprises a nucleotide sequence at least 80% identical, at least 85%, at least 90%, at least 92%, at least 95%, at least 97%, or at least 99% to SEQ ID NO: 12 (Ad vector with sequence encoding ma modified Brachyury antigen), positions 1033-2083 of SEQ ID NO: 12, or SEQ ID NO: 42.

10. The composition of claim 4, wherein the MUC1 antigen comprises a sequence at least 80%, at least 85%, at least 90%, at least 92%, at least 95%, at least 97%, or at least 99% identical to SEQ ID NO: 10 or SEQ ID NO: 41.

11. The composition of claim 4, wherein the third nucleic acid sequence encoding a MUC1 antigen comprises at least 80%, at least 85%, at least 90%, at least 92%, at least 95%, at least 97%, or at least 99% identity to SEQ ID NO: 8, SEQ ID NO: 9, or SEQ ID NO: 41.

12. The composition of claim 4, wherein the MUC-1 antigen binds to HLA-A2, HLA-A3, HLA-A24, or a combination thereof.

13. The composition of claim 4, wherein the replication-defective virus vector, the second replication-defective virus vector, and/or the third replication-defective virus vector is an adenovirus vector.

14. The composition of claim 13, wherein the adenovirus vector comprises a deletion in an E1 region, an E2b region, an E3 region, an E4 region, or a combination thereof.

15. (canceled)

16. (canceled)

17. The composition of claim 1, wherein the composition comprises from at least 1×10⁹ virus particles to at least 5×10¹² virus particles.

18. (canceled)

19. (canceled)

20. (canceled)

21. (canceled)

22. The composition of claim 1, wherein the composition or the replication-defective virus vector further comprises a nucleic acid sequences encoding a costimulatory molecule.

23. The composition of claim 22, wherein the costimulatory molecule comprises B7, ICAM-1, LFA-3, or a combination thereof.

24. (canceled)

25. The composition of claim 1, wherein the composition further comprises a plurality of nucleic acid sequences encoding a plurality of costimulatory molecules positioned in the same replication-defective virus vector.

26. The composition of claim 1, wherein the composition further comprises a plurality of nucleic acid sequences encoding a plurality of costimulatory molecules positioned in separate replication-defective virus vectors.

27. The composition of claim 1, wherein the composition further comprises a nucleic acid sequence encoding one or more additional target antigens or immunological epitopes thereof.

28. The composition of claim 1, wherein the replication-defective virus vector further comprises a nucleic acid sequence encoding one or more additional target antigens or immunological epitopes thereof.

29. (canceled)

30. The composition of claim 27, wherein the one or more additional target antigens is CEA, folate receptor alpha, WT1, HPV E6, HPV E7, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, PSCA, PSMA, PAP, Tyrosinase, TRP-1, TRP-2, ART-4, CAMEL, Cyp-B, Her2/neu, BRCA1, BRACHYURY, BRACHYURY (TIVS7-2, polymorphism), BRACHYURY (IVS7 T/C polymorphism), T BRACHYURY, T, hTERT, hTRT, iCE, MUC1, MUC1 (VNTR polymorphism), MUC1c, MUC1n, MUC2, PRAME, P15, RU1, RU2, SART-1, SART-3, WT1, AFP, β-catenin/m, Caspase-8/m, CDK-4/m, Her2/neu, Her3, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, or TEL/AML1, or a modified variant, a splice variant, a functional epitope, an epitope agonist, or a combination thereof.

31. (canceled)

32. (canceled)

33. (canceled)

34. (canceled)

35. (canceled)

36. The composition of claim 1, wherein the replication-defective virus vector further comprises a selectable marker.

37. (canceled)

38. A composition comprising one or more replication-defective virus vectors comprising a nucleic acid sequence encoding a prostate specific antigen (PSA), a nucleic acid sequence encoding prostate-specific membrane antigen (PSMA), a nucleic acid sequence encoding a Brachyury antigen, a nucleic acid sequence encoding a MUC1 antigen, or a combination thereof.

39. (canceled)

40. (canceled)

41. A composition comprising one or more replication-defective virus vectors comprising a nucleic acid sequence encoding a prostate specific antigen (PSA), a nucleic acid sequence encoding prostate-specific membrane antigen (PSMA), a nucleic acid sequence encoding a Brachyury antigen, a nucleic acid sequence encoding a MUC1 antigen, and a nucleic acid sequence encoding a CEA antigen.

42. (canceled)

43. A pharmaceutical composition comprising the composition according to claim 1 and a pharmaceutically acceptable carrier.

44. A host cell comprising the composition according to claim 1.

45. A method of preparing a tumor vaccine, the method comprising preparing a pharmaceutical composition according to claim 43.

46. A method of enhancing an immune response in a subject in need thereof, the method comprising administering a therapeutically effective amount of the composition of claim 1 to the subject.

47. A method of treating a PSA-expressing or PSMA-expressing cancer in a subject in need thereof, the method comprising administering a therapeutically effective amount of the composition of claim 1 to the subject.

48.-88. (canceled)


SEQUENCE LISTING (113 sequences)

SEQ ID NO: 1 - protein, 261 aa, Artificial Sequence
  Description of Artificial Sequence Synthetic polypeptide
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFTHPLYDMSLLKNRFLRPGDDSSHDLMLLRLSEPAELTDAMKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - DNA, 783 bp, Artificial Sequence
  Description of Artificial Sequence Synthetic polynucleotide
atgtgggtgcccgtggtgttcctgaccctgagcgtgacctggatcggcgccgcccccctgatcctgagcaggatcgtgggcggctgggagtgcgagaagcacagccagccctggcaggtgctggtggccagcaggggcagggccgtgtgcggcggcgtgctggtgcacccccagtgggtgctgaccgccgcccactgcatcaggaacaagagcgtgatcctgctgggcaggcacagcctgttccaccccgaggacaccggccaggtgttccaggtgagccacagcttcacccaccccctgtacgacatgagcctgctgaagaacaggttcctgaggcccggcgacgacagcagccacgacctgatgctgctgaggctgagcgagcccgccgagctgaccgacgccatgaaggtgatggacctgcccacccaggagcccgccctgggcaccacctgctacgccagcggctggggcagcatcgagcccgaggagttcctgacccccaagaagctgcagtgcgtggacctgcacgtgatcagcaacgacgtgtgcgcccaggtgcacccccagaaggtgaccaagttcatgctgtgcgccggcaggtggaccggcggcaagagcacctgcagcggcgacagcggcggccccctggtgtgcaacggcgtgctgcagggcatcaccagctggggcagcgagccctgcgccctgcccgagaggcccagcctgtacaccaaggtggtgcactacaggaagtggatcaaggacaccatcgtggccaacccc

SEQ ID NO: 3 - DNA, 2500 bp, Artificial Sequence
  Description of Artificial Sequence Synthetic polynucleotide
ggaggacacttctcagaaggggttgttttgcttttgcttatttccgtccatttccctctctgcgcgcggaccttccttttccagatggtgagagccgcggggacacccgacgccggggcaggctgatccacgatcctgggtgtgcgtaacgccgcctggggctccgtgggcgagggacgtgtggggacaggtgcaccggaaactgccagactggagagttgaggcatcggaggcgcgagaacagcactactactgcggcgagacgagcgcggcgcatcccaaagcccggccaaatgcgctcgtccctgggaggggagggaggcgcgcctggagcggggacagtcttggtccgcgccctcctcccgggtctgtgccgggacccgggacccgggagccgtcgcaggtctcggtccaaggggccccttttctcggaagggcggcggccaagagcagggaaggtggatctcaggtagcgagtctgggcttcggggacggcggggaggggagccggacgggaggatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaagctttccttgatgcaaaggaaagaagtgatcacaaagagatgatggaggaacccggagacagccagcaacctgggtactcccaatgggggtggcttcttcctggaaccagcaccctgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgaagcagcaaggcccaggtcccgaaagatgcagtgactttttgtcgtggcagccagtggtgactggattgacctactaggtacccagtggcagtctcaggttaagaaggaaatgcagcctcagtaacttccttttcaaagcagtggaggagcacacggcacctttccccagagccccagcatcccttgctcacacctgcagtagcggtgctgtcccaggtggcttacagatgaacccaactgtggagatgatgcagttggcccaacctcactgacggtgaaaaaatgtttgccagggtccagaaactttttttggtttatttctcatacagtgtattggcaactttggcacaccagaatttgtaaactccaccagtcctactttagtgagataaaaagcacactcttaatcttcttccttgttgctttcaagtagttagagttgagctgttaaggacagaataaaatcatagttgaggacagcaggttttagttgaattgaaaatttgactgctctgccccctagaatgtgtgtattttaagcatatgtagctaatctcttgtgttgttaaactataactgtttcatatttttcttttgacaaagtagccaaagacaatcagcagaaagcattttctgcaaaataaacgcaatatgcaaaaaaaaaaaaaaaaaa

SEQ ID NO: 4 - DNA, 1251 bp, Artificial Sequence
  Description of Artificial Sequence Synthetic polynucleotide
tctagagccaccatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgctagaagtgatcacaaagagatgatggaggaacccggagacagccagcaacctgggtactcccaatgggggtggcttcttcctggaaccagcaccgtgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgagatatc

SEQ ID NO: 5 - protein, 410 aa, Artificial Sequence
  Description of Artificial Sequence Synthetic polypeptide
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTARSDHKEMMEEPGDSQQPGYSQWGWLLPGTSTVCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 6 - protein, 435 aa, Artificial Sequence
  Description of Artificial Sequence Synthetic polypeptide
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGDPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVTNGLGAQFFRGSPAHYTPLTHPVSAPSSSGSPLYEGAAAATNIVDSQYDAAAQGRLIASWTPVSPPSM

SEQ ID NO: 7 - protein, 9 aa, Artificial Sequence
  Description of Artificial Sequence Synthetic peptide
WLLPGTSTV

SEQ ID NO: 8 - DNA, 1826 bp, Artificial Sequence
  Description of Artificial Sequence Synthetic polynucleotide
cgctccacctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 9 - DNA, 1826 bp, Artificial Sequence
  Description of Artificial Sequence Synthetic polynucleotide
cgctccacctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccctttggggacaggatgtcacctcggtcccagtcaccaggccagccctgggctccaccaccccgccagcccacgatgtcacctcagccccggacaacaagccagccccgggctccaccgcccccccagcccacggtgtcacctcgtatcttgacaccaggccggccccggtttatcttgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggtttatctggccattgtctatctcattgccttggctgtcgctcaggttcgccgaaagaactacgggcagctggacatctttccagcccgggataaataccatcctatgagcgagtacgctctttaccacacccatgggcgctatgtgccccctagcagtcttttccgtagcccctatgagaaggtttctgcaggtaatggtggcagctatctctcttacacaaacccagcagtggcagccgcttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 10 - protein, 475 aa, Artificial Sequence
  Description of Artificial Sequence Synthetic polypeptide
MTPGTQSPFFLLLLLTVLTVVTGSGHASSTPGGEKETSATQRSSVPSSTEKNAVSMTSSVLSSHSPGSGSSTTQGQDVTLAPATEPASGSAALWGQDVTSVPVTRPALGSTTPPAHDVTSAPDNKPAPGSTAPPAHGVTSYLDTRPAPVYLAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVYLAIVYLIALAVAQVRRKNYGQLDIFPARDKYHPMSEYALYHTHGRYVPPSSLFRSPYEKVSAGNGGSYLSYTNPAVAAASANL

SEQ ID NO: 11 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 12 - DNA, 31465 bp, Artificial Sequence
  Description of Artificial Sequence Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttctagatgcatgctcgagcggccgccagtgtgatggatatctgcagaattcgcccttgcttctagagccaccatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgctagaagtgatcacaaagagatgatggaggaacccggagacagccagcaacctgggtactcccaatgggggtggcttcttcctggaaccagcaccgtgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 13 - protein, 9 aa, Artificial Sequence
  Description of Artificial Sequence Synthetic peptide
EQVWGMAVR

SEQ ID NO: 14 - protein, 15 aa, Artificial Sequence
  Description of Artificial Sequence Synthetic peptide
CQGPEQVWGMAVREL

SEQ ID NO: 15 - protein, 9 aa, Artificial Sequence
  Description of Artificial Sequence Synthetic peptide
GETVTMPCP

SEQ ID NO: 16 - protein, 15 aa, Artificial Sequence
  Description of Artificial Sequence Synthetic peptide
NVGETVTMPCPKVFS

SEQ ID NO: 17 - protein, 9 aa, Artificial Sequence
  Description of Artificial Sequence Synthetic peptide
GLGAQCSEA

SEQ ID NO: 18 - protein, 15 aa, Artificial Sequence
  Description of Artificial Sequence Synthetic peptide
NNGLGAQCSEAVTLN

SEQ ID NO: 19 - protein, 9 aa, Artificial Sequence
  Description of Artificial Sequence Synthetic peptide
RKLTTELTI

SEQ ID NO: 20 - protein, 15 aa, Artificial Sequence
  Description of Artificial Sequence Synthetic peptide
LGPERRKLTTELTII

SEQ ID NO: 21 - protein, 9 aa, Artificial Sequence
  Description of Artificial Sequence Synthetic peptide
PERRKLTTE

SEQ ID NO: 22 - protein, 9 aa, Artificial Sequence
  Description of Artificial Sequence Synthetic peptide
MDWVWMDTT

SEQ ID NO: 23 - protein, 15 aa, Artificial Sequence
  Description of Artificial Sequence Synthetic peptide
AVMDWVWMDTTLSLS

SEQ ID NO: 24 - protein, 9 aa, Artificial Sequence
  Description of Artificial Sequence Synthetic peptide
VWMDTTLSL

SEQ ID NO: 25 - protein, 9 aa, Artificial Sequence
  Description of Artificial Sequence Synthetic peptide
GKTLNPSQT

SEQ ID NO: 26 - protein, 15 aa, Artificial Sequence
  Description of Artificial Sequence Synthetic peptide
SWFREGKTLNPSQTS

SEQ ID NO: 27 - protein, 9 aa, Artificial Sequence
  Description of Artificial Sequence Synthetic peptide
REGKTLNPS

SEQ ID NO: 28 - protein, 9 aa, Artificial Sequence
  Description of Artificial Sequence Synthetic peptide
VRNATSYRC

SEQ ID NO: 29 - protein, 15 aa, Artificial Sequence
  Description of Artificial Sequence Synthetic peptide
LPNVTVRNATSYRCG

SEQ ID NO: 30 - protein, 9 aa, Artificial Sequence
  Description of Artificial Sequence Synthetic peptide
NVTVRNATS

SEQ ID NO: 31 - protein, 9 aa, Artificial Sequence
  Description of Artificial Sequence Synthetic peptide
FAMAQIPSL

SEQ ID NO: 32 - protein, 15 aa, Artificial Sequence
  Description of Artificial Sequence Synthetic peptide
PFAMAQIPSLSLRAV

SEQ ID NO: 33 - protein, 9 aa, Artificial Sequence
  Description of Artificial Sequence Synthetic peptide
AQIPSLSLR

SEQ ID NO: 34 - protein, 261 aa, Artificial Sequence
  Description of Artificial Sequence Synthetic polypeptide
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFTHPLYDMSLLKNRFLRPGDDSSHDHMLLRLSEPAELTDAMKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 35 - DNA, 786 bp, Artificial Sequence
  Description of Artificial Sequence Synthetic polynucleotide
atgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcacacacccgctctacgatatgagcctcctgaagaatcgattcctcaggccaggtgatgactccagccacgaccacatgctgctccgcctgtcagagcctgccgagctcacggatgctatgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctga

SEQ ID NO: 36 - DNA, 2253 bp, Artificial Sequence
  Description of Artificial Sequence Synthetic polynucleotide
atgtggaacctgctgcacgagacagacagcgccgtggccacagctagaaggcccagatggctgtgtgctggcgctctggtgctggctggcggctttttcctgctgggcttcctgttcggctggttcatcaagagcagcaacgaggccaccaacatcacccccaagcacaacatgaaggcctttctggacgagctgaaggccgagaatatcaagaagttcctgtacaacttcacccagatcccccacctggccggcaccgagcagaatttccagctggccaagcagatccagagccagtggaaagagttcggcctggacagcgtggaactggcccactacgatgtgctgctgagctaccccaacaagacccaccccaactacatcagcatcatcaacgaggacggcaacgagattttcaacaccagcctgttcgagccccctccacccggctacgagaacgtgtccgatattgtgcccccattcagcgcattcagtccacaaggcatgcccgagggcgacctggtgtacgtgaactacgcccggaccgaggacttcttcaagctggaacgggacatgaagatcaactgcagcggcaagatcgtgatcgccagatacggcaaggtgttccggggcaacaaagtgaagaacgcccagctggcaggcgccaagggcgtgatcctgtactccgaccccgccgactatttcgcccctggcgtgaagtcctaccccgacggctggaatctgcctggcggcggagtgcagagaggcaacatcctgaacctgaacggcgctggcgaccctctgacacctggctaccctgccaacgagtacgcctacagacggggaattgccgaggccgtgggcctgccttctatccccgtgcaccctatcggctactacgacgcccagaaactgctggaaaagatgggcggcagcgcccctcctgacagctcttggagaggcagcctgaaggtgccctacaacgtgggcccaggcttcaccggcaacttcagcacccagaaagtgaaaatgcacatccacagcaccaacgaagtgacccggatctacaatgtgatcggcaccctgagaggcgccgtggaacccgacagatatgtgatcctgggcggccacagagacagctgggtgttcggcggaatcgaccctcagtctggcgccgctgtggtgcacgagatcgtgcggtctttcggcacactgaagaaagagggatggcggcccagacggaccatcctgttcgcctcttgggacgccgaggaattcggcctgctgggatctaccgagtgggccgaggaaaacagcagactgctgcaggaacggggcgtggcctacatcaacgccgacagcagcatcgagggcaactacaccctgcgggtggactgcacccccctgatgtatagcctggtgcacaacctgaccaaagagctgaagtcccccgacgagggctttgagggcaagagcctgtacgagagctggaccaagaagtcccctagccccgagttcagcggcatgcccagaatcagcaagctgggctccggcaacgacttcgaggtgttcttccagcggctgggaatcgccagcggcagagccagatacaccaagaactgggagacaaacaagttctccggctaccccctgtaccacagcgtgtacgagacatacgagctggtggaaaagttctacgaccccatgttcaagtaccacctgaccgtggcccaagtgcggggaggcatggtgttcgaactggccaacagcatcgtgctgcccttcgactgcagagactacgccgtggtgctgcggaagtacgccgacaaaatctacagcatcagcatgaagcacccccaggaaatgaagacctacagcgtgtccttcgactccctgttctccgccgtgaagaatttcaccgagatcgccagcaagttcagcgagcggctgcaggacttcgacaagagcaaccctatcgtgctgaggatgatgaacgaccagctgatgttcctggaacgcgccttcatcgaccccctgggactgcccgacagacccttctaccggcacgtgatctatgcccccagcagccacaacaaatacgccggcgagagcttccccggcatctacgatgccctgttcgacatcgagagcaaggtggaccccagcaaggcctggggcgaagtgaagcggcaaatctacgtggccgcattcacagtgcaggccgctgccgagacactgtctgaagtggcttga

SEQ ID NO: 37 - DNA, 2109 bp, Homo sapiens
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 38 - protein, 9 aa, Homo sapiens
YLSGADLNL

SEQ ID NO: 39 - protein, 9 aa, Homo sapiens
YLSGANLNL

SEQ ID NO: 40 - DNA, 32315 bp, Artificial Sequence
  Description of Artificial Sequence Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccgagctcggatccactagtaacggccgccagtgtgctggaattcggcttaaaggtacccagagcagacagccgccaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 41 - DNA, 1428 bp, Homo sapiens
atgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccctttggggacaggatgtcacctcggtcccagtcaccaggccagccctgggctccaccaccccgccagcccacgatgtcacctcagccccggacaacaagccagccccgggctccaccgcccccccagcccacggtgtcacctcgtatcttgacaccaggccggccccggtttatcttgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggtttatctggccattgtctatctcattgccttggctgtcgctcaggttcgccgaaagaactacgggcagctggacatctttccagcccgggataaataccatcctatgagcgagtacgctctttaccacacccatgggcgctatgtgccccctagcagtcttttccgtagcccctatgagaaggtttctgcaggtaatggtggcagctatctctcttacacaaacccagcagtggcagccgcttctgccaacttgtag

SEQ ID NO: 42 - DNA, 1233 bp, Homo sapiens
atgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgctagaagtgatcacaaagagatgatggaggaacccggagacagccagcaacctgggtactcccaatgggggtggcttcttcctggaaccagcaccgtgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtga

SEQ ID NO: 43 - protein, 132 aa, Mycobacterium tuberculosis
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 44 - protein, 230 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFDDDDKDPPDPHQPDMTKGYCPGGRWGFGDLAVCDGEKYPDGSFWHQWMQTWFTGPQFYFDCVSGGEPLPGPPPPGGCGGAIPSEQPNAP

SEQ ID NO: 45 - protein, 578 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFPLVPRGSPMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYSGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 46 - protein, 220 aa, Artificial Sequence [sequence omitted]

SEQ ID NO: 47 - protein, 729 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 48 - protein, 30 aa, Artificial Sequence
  Description of Artificial Sequence Synthetic polypeptide
TAASDNFQLSQGGQGFAIPIGQAMAIAGQI

SEQ ID NO: 49 - protein, 128 aa, Artificial Sequence
  Description of Artificial Sequence Synthetic polypeptide
TAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 50 - protein, 128 aa, Mycobacterium tuberculosis
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAE

SEQ ID NO: 51 - protein, 355 aa, Mycobacterium tuberculosis
MSNSRRRSLRWSWLLSVLAAVGLGLATAPAQAAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 52 - protein, 364 aa, Haemophilus influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPAFFTDVNQMYDVLLNKSGATGVFTDFPDTGVEFLKGIK

SEQ ID NO: 53 - protein, 313 aa, Streptococcus pneumonae
MEINVSKLRTDLPQVGVQPYRQVHAHSTGNPHSTVQNEADYHWRKDPELGFFSHIVGNGCIMQVGPVDNGAWDVGGGWNAETYAAVELIESHSTKEEFMTDYRLYIELLRNLADEAGLPKTLDTGSLAGIKTHEYCTNNQPNNHSDHVDPYPYLAKWGISREQFKHDIENGLTIETGWQKNDTGYWYVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPEFRMSQMA

SEQ ID NO: 54 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 55 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 56 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 57 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 58 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 59 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 60 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 61 - protein, 152 aa, Homo sapiens
MSRLPVLLLLQLLVRPGLQAPMTQTTSLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 62 - protein, 153 aa, Homo sapiens [sequence omitted]

SEQ ID NO: 63 - protein, 134 aa, Homo sapiens
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 64 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 65 - protein, 140 aa, Homo sapiens
MVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 66 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 67 - protein, 145 aa, Homo sapiens [sequence omitted]

SEQ ID NO: 68 - protein, 136 aa, Homo sapiens [sequence omitted]

SEQ ID NO: 69 - protein, 656 aa, Homo sapiens
MEGDGSDPEPPDAGEDSKSENGENAPIYCICRKPDINCFMIGCDNCNEWFHGDCIRITEKMAKAIREWYCRECREKDPKLEIRYRHKKSRERDGNERDSSEPRDEGGGRKRPVPDPNLQRRAGSGTGVGAMLARGSASPHKSSPQPLVATPSQHHQQQQQQIKRSARMCGECEACRRTEDCGHCDFCRDMKKFGGPNKIRQKCRLRQCQLRARESYKYFPSSLSPVTPSESLPRPRRPLPTQQQPQPSQKLGRIREDEGAVASSTVKEPPEATATPEPLSDEDLPLDPDLYQDFCAGAFDDNGLPWMSDTEESPFLDPALRKRAVKVKHVKRREKKSEKKKEERYKRHRQKQKHKDKWKHPERADAKDPASLPQCLGPGCVRPAQPSSKYCSDDCGMKLAANRIYEILPQRIQQWQQSPCIAEEHGKKLLERIRREQQSARTRLQEMERRFHELEAIILRAKQQAVREDEESNEGDSDDTDLQIFCVSCGHPINPRVALRHMERCYAKYESQTSFGSMYPTRIEGATRLFCDVYNPQSKTYCKRLQVLCPEHSRDPKVPADEVCGCPLVRDVFELTGDFCRLPKRQCNRHYCWEKLRRAEVDLERVRVWYKLDELFEQERNVRTAMTNRAGLLALMLHQTIQHDPLTTDLRSSADR

SEQ ID NO: 70 - protein, 124 aa, Vibrio cholerae
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 71 - protein, 258 aa, Vibrio cholerae
MVKIIFVFFIFLSSFSYANDDKLYRADSRPPDEIKQSGGLMPRGQNEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 72 - protein, 124 aa, Vibrio cholerae
MIKLKFGVFFTVLLSSAYAHGTPQNITDLCAEYHNTQIHTLNDKILSYTESLAGNREMAIITFKNGATFQVEVPGSQHIDSQKKAIERMKDTLRIAYLTEAKVEKLCVWNNKTPHAIAAISMAN

SEQ ID NO: 73 - protein, 18 aa, Homo sapiens
DPNAPKRPPSAFFLFCSE

SEQ ID NO: 74 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 75 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 76 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 77 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 78 - protein, 7 aa, Artificial Sequence
  Description of Artificial Sequence Synthetic peptide
QEINSSY

SEQ ID NO: 79 - protein, 7 aa, Artificial Sequence
  Description of Artificial Sequence Synthetic peptide
SHPRLSA

SEQ ID NO: 80 - protein, 7 aa, Artificial Sequence
  Description of Artificial Sequence Synthetic peptide
SMPNPMV

SEQ ID NO: 81 - protein, 7 aa, Artificial Sequence
  Description of Artificial Sequence Synthetic peptide
GLQQVLL

SEQ ID NO: 82 - protein, 7 aa, Artificial Sequence
  Description of Artificial Sequence Synthetic peptide
HELSVLL

SEQ ID NO: 83 - protein, 7 aa, Artificial Sequence
  Description of Artificial Sequence Synthetic peptide
YAPQRLP

SEQ ID NO: 84 - protein, 7 aa, Artificial Sequence
  Description of Artificial Sequence Synthetic peptide
TPRTLPT

SEQ ID NO: 85 - protein, 7 aa, Artificial Sequence
  Description of Artificial Sequence Synthetic peptide
APVHSSI

SEQ ID NO: 86 - protein, 7 aa, Artificial Sequence
  Description of Artificial Sequence Synthetic peptide
APPHALS

SEQ ID NO: 87 - protein, 7 aa, Artificial Sequence
  Description of Artificial Sequence Synthetic peptide
TFSNRFI

SEQ ID NO: 88 - protein, 7 aa, Artificial Sequence
  Description of Artificial Sequence Synthetic peptide
VVPTPPY

SEQ ID NO: 89 - protein, 7 aa, Artificial Sequence
  Description of Artificial Sequence Synthetic peptide
ELAPDSP

SEQ ID NO: 90 - protein, 69 aa, Shigella dysenteria 1
TPDCVTGKVEYTKYNDDDTFTVKVGDKELFTNRWNLQSLLLSAQITGMTVTIKQNACHNGGGFSEVIFR

SEQ ID NO: 91 - protein, 560 aa, Corynephage omega
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 92 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 93 - protein, 297 aa, Artificial Sequence
  Description of Artificial Sequence Synthetic polypeptide
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 535 aa, Corynebacterium diphtheriae
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 95 - protein, 1331 aa, Homo sapiens [sequence omitted]

SEQ ID NO: 96 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 97 - protein, 476 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 98 - protein, 234 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 99 - protein, 11 aa, Artificial Sequence
  Description of Artificial Sequence Synthetic peptide
MAVPMQLSCSR

SEQ ID NO: 100 - protein, 4 aa, Artificial Sequence
  Description of Artificial Sequence Synthetic peptide
RSTG

SEQ ID NO: 101 - protein, 2 aa, Artificial Sequence
  Description of Artificial Sequence Synthetic peptide
TR

SEQ ID NO: 102 - protein, 3 aa, Artificial Sequence
  Description of Artificial Sequence Synthetic peptide
RSQ

SEQ ID NO: 103 - protein, 5 aa, Artificial Sequence
  Description of Artificial Sequence Synthetic peptide
RSAGE

SEQ ID NO: 104 - protein, 2 aa, Artificial Sequence
  Description of Artificial Sequence Synthetic peptide
RS

SEQ ID NO: 105 - protein, 2 aa, Artificial Sequence
  Description of Artificial Sequence Synthetic peptide
GG

SEQ ID NO: 106 - protein, 9 aa, Artificial Sequence
  Description of Artificial Sequence Synthetic peptide
GSGGSGGSG

SEQ ID NO: 107 - protein, 11 aa, Artificial Sequence
  Description of Artificial Sequence Synthetic peptide
GGSGGSGGSGG

SEQ ID NO: 108 - protein, 14 aa, Artificial Sequence
  Description of Artificial Sequence Synthetic peptide
GGSGGSGGSGGSGG

SEQ ID NO: 109 - protein, 17 aa, Artificial Sequence
  Description of Artificial Sequence Synthetic peptide
GGSGGSGGSGGSGGSGG

SEQ ID NO: 110 - protein, 20 aa, Artificial Sequence
  Description of Artificial Sequence Synthetic peptide
GGSGGSGGSGGSGGSGGSGG

SEQ ID NO: 111 - protein, 23 aa, Artificial Sequence
  Description of Artificial Sequence Synthetic peptide
GGSGGSGGSGGSGGSGGSGGSGG

SEQ ID NO: 112 - protein, 16 aa, Artificial Sequence
  Description of Artificial Sequence Synthetic peptide
GGSGGSGGSGGSGGSG

SEQ ID NO: 113 - protein, 16 aa, Artificial Sequence
  Description of Artificial Sequence Synthetic peptide
GSGGSGGSGGSGGSGG